(19) United States
(12) Patent Application Publication
(10) Pub. No.: US 2013/0345268 A1
(43) Pub. Date: Dec. 26, 2013

(54) Methods and Compositions for the Treatment of RAS Associated Disorders

(71) Applicants: The Trustees of Dartmouth College, Hanover, NH (US); Children's Hospital Medical Center, Cincinnati, OH (US)

(72) Inventors: Nancy Ratner, Cincinnati, OH (US); Yolanda Sanchez, Orford, NH (US); Gunnar Johansson, Taipei (TW); William Seibel, Hamilton, OH (US)

(73) Assignees: The Trustees of Dartmouth College, Hanover, NH (US); Children's Hospital Medical Center, Cincinnati, OH (US)

(21) Appl. No.: 13/970,836

(22) Filed: Aug. 20, 2013

Related U.S. Application Data
(63) Continuation-in-part of application No. 13/208,777, filed on Aug. 12, 2011.
Application No. 13/208,777 is a continuation-in-part of application No. PCT/US10/24237, filed on Feb. 15, 2010.
(60) Provisional application No. 61/152,453, filed on Feb. 13, 2009.

(51) Int. Cl. C07D 339/08; C07D 277/34

(52) U.S. Cl. CPC C07D 339/08; C07D 277/34
USPC 514/369; 514/436

(57) ABSTRACT

The instant disclosure relates to compositions that may be useful as therapeutic agents for the treatment of disorders associated or caused by Ras deregulation or dysregulation, for example, disorders associated with alterations in the NF1 gene such as neurofibromatosis type I, fungal infections such as those caused by Candida albicans, and proliferative disorders such as glioblastoma.

### CROSS REFERENCE TO RELATED APPLICATIONS

This application is a continuation in part of U.S. patent application Ser. No. 13/208,777, filed Aug. 11, 2011, which is a continuation in part of International Application No. PCT/US10/24237, filed Feb. 15, 2010, which in turn claims priority to U.S. Provisional Application Ser. No. 61/152,453, filed Feb. 13, 2009, entitled “Methods and Compounds for the Treatment of NF1 Related Disorders,” all of which are hereby incorporated by reference in its entirety.

### SUMMARY OF THE INVENTION

Disclosed herein are agents that may be useful for the treatment of disorders associated with deregulation or dysregulation of Ras and/or for the treatment and/or prevention of Neurofibromatosis Type I or NF1-related disorders or conditions. Methods of identifying such compounds are further disclosed

### DESCRIPTION OF THE DRAWINGS

FIG. 1A. GRD adenovirus blocks Ras/Map-kinase signaling in human NF1 MPNST cell lines.

FIG. 1B. In vitro growth of MPNST cell lines at Days 1 and 4.

FIG. 2. Cell Growth in NF1+/+ and NFI−/− MPNST cell lines in the presence of increasing concentrations of the compound of Formula 1a.

FIG. 3. Inhibition of growth of cells lacking Ira2 and Erg6 by the compound of Formula Ia. Strains were grown overnight in Synthetic Complete media. 75 μL of culture at a starting optical density of 0.1 were added to a 96 well plate containing 75 μL of Synthetic Complete media with twice the desired final concentration of compound in 1% DMSO. Plates were incubated in a humidified 32C chamber, and scanned on an optical density plate reader at 595 nm after 24 h. The bars represent the mean of three wells, and the error bars represent ±2× standard error.

FIG. 4 depicts the effects of two compounds active in NF1−/− MPNST cells on the growth of U-87 MG GBM (glioblastoma) cells in vitro. FIG. 4a depicts the effects of the compound of Formula I(a); FIG. 4b depicts the effects of the compound of Formula IV(c). U87-MG cells were seeded in 96 well plates at a density of 1000 per well in in DMEM containing 10% fetal bovine serum. The next day, medium was removed and fresh medium containing 0.1% DMSO or compound was added back. Plates were incubated for three days at 37° C., 5% CO₂. Cell growth was assessed with Alamar Blue with values normalized to the DMSO-treated wells. Values are mean of three wells. The AC50 for the compound of Formula Ia was 3.77 micromolar +/−0.7. The AC50 for the compound of Formula IV(c) was 0.97 micromolar +/−0.1. AC50 was calculated using data from three independent experiments.

### DETAILED DESCRIPTION

Abbreviations—Malignant Peripheral Nerve Sheath Tumor (MPNST); NF1 (with italics) indicates the NF1 gene; NF1 (without italics) indicates Neurofibromatosis Type 1; loss of heterozygosity region of the NF1 gene (LOH); mitogen glial growth factor (GGF); Normal Human Schwann Cells (NHSC); The identifier “Δ” indicates a change in gene status that results in a loss of function via deletion or mutation. For example, as used herein, the term “Erg6Δ” reflects a cell having a mutated or deleted Erg6 gene, such that Erg6 gene product function is impaired. As used herein, the use of italics generally indicates the gene, as compared to the gene product, which is not italicized.

For convenience, certain terms employed in the specification, examples and claims are collected here. These definitions should be read in light of the remainder of the disclosure and understood as by a person of skill in the art. Unless defined otherwise, all technical and scientific terms used herein have the same meaning as commonly understood by a person of ordinary skill in the art. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the materials and/or methodologies which are reported in the publications which might be used in connection with the invention.

Nothing herein is to be construed as an admission that the invention is not entitled to antedate such disclosure by virtue of prior invention. In order to provide a clear and consistent understanding of the specification and claims, including the scope to be given such terms, the following definitions are provided:

The articles “a” and “an” refer to one or to more than one (i.e., to at least one) of the grammatical object of the article. By way of example, “an element” means one element or more than one element.

The term “alteration” is intended to encompass any mutation or deletion of a gene, including truncation, deletion of the entire sequence or a portion of the gene, or one or more mutations that result in ablated or significantly attenuated gene function, “loss of function,” such that the net result of the alteration is to essentially or substantially reduce the function of a gene of interest such that the assay as described herein can be effectively carried out to identify potential therapeutic agents. The term may also encompass any mutation that results in suppression or altered translation or transcription of the gene of interest, such that the gene function is essentially or substantially reduced in function. Determination of alterations with respect to a particular gene that satisfies the above-definition may be determined via routine experimentation.

“Biologically acceptable medium” includes any and all solvents, dispersion media, and the like which may be appropriate for the desired route of administration of the pharmaceutical preparation. The use of such media for pharmaceutically active substances is known in the art.

The terms “candidate agent” or “candidate compound” or “candidate molecule” or “candidate drug” may be used interchangeably and encompass an agent, compound, or molecule which has the potential to have a therapeutic effect in vivo or in vitro which can be used with the disclosed methods to determine whether the agent or compound has a desired biological or biochemical activity.

The phrase “cellular characteristic associated with a proliferative disorder” as used herein is intended to include any feature or property, whether biological or biochemical, of a cell or cellular population that may be indicative of a proliferative disorder, particularly that of NF1 or an NF1 related disease. For example, the characteristic may include migration, proliferation, rate of cell growth, or cellular adhesion. The cellular characteristic may be that of individual cells or a population of cells.

“Chemical library” or “compound library” generally refers to a collection of stored chemicals often used in high-throughput screening or industrial manufacture. The library may comprise a series of stored chemicals, each chemical typically having associated information stored in a database. The associated information may include, for example, the chemical structure, purity, quantity, and physiochemical characteristics of the compound. Chemical or compound libraries may focus on large groups of varied organic chemical series such that an organic chemist can make many variations on the same molecular scaffold or molecular backbone. Chemicals may also be purchased from outside vendors as well and included into an internal chemical library.

The term “compound” (e.g., as in “candidate compound”) includes both exogenously added candidate compounds and peptides endogenously expressed from a peptide library. For example, in certain aspects, the reagent cell may produce the candidate compound being screened. For instance, the reagent cell can produce. e.g., a candidate polypeptide, a candidate nucleic acid and/or a candidate carbohydrate which may be screened for its ability to modulate the receptor/channel activity. In such aspects, a culture of such reagent cells will collectively provide a library of potential effector molecules and those members of the library which either agonize or antagonize the receptor or ion channel function can be selected and identified. Moreover, it will be apparent that the reagent cell can be used to detect agents which transduce a signal via the receptor or channel of interest.

The phrase “disorder associated with Ras deregulation or dysregulation” includes diseases wherein the etiology the disorder involves deregulation of RAS signaling, for example, wherein RAS activity may be increased to the extent that a disease state arises. The Ras forms contemplated herein encompass any known variant of Ras and include K-Ras (for example, NCBI Accession Number NG 007524) (having two splice variants), H-Ras (for example, NCBI Accession Number NG 007666), and N-Ras (for example, NCBI Accession Number NG 007572), and R-Ras (for example, NCBI Accession Number NC_{—}000019 (Gene ID 6237), Ras 1, Ras 2 and combinations thereof. The disorder associated with Ras deregulation or dysregulation may be a proliferative disorder such as cancer. The disorder associated with Ras deregulation or dysregulation may be Neurofibromatosis Type 1; a disease state that results from a mutation or loss of function in the NF1 gene (SEQ ID NO: 22); neuroblastoma, lung adenocarcinoma, squamous cell carcinoma, glioblastomas, ovarian cancer, colon cancer; lung cancer; neurofibromas, malignant peripheral nerve sheath tumors, optic gliomas, Schwannomas, gliomas, leukemias, pheochromocytomas, pancreatic cancer; pancreatic adenocarcinoma (wherein greater than about 90% of tumors have activating mutations in K-Ras), and/or other sporadic cancers, and may also include non-tumor manifestations such as learning disorders or and fungal infections such as those involving the transformation of fungi to the invasive hyphal form. In one aspect, the disorder may comprise a disorder caused by Candida albicans.

The terms “drug,” “pharmaceutically active agent,” “bioactive agent,” “therapeutic agent,” and “active agent” may be used interchangeably and means a substance, such as a chemical compound or complex, that has a measurable beneficial physiological effect on the body, such as a therapeutic effect in treatment of a disease or disorder, when administered in an effective amount. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

A “hit” means as a candidate agent, compound, or molecule inhibiting growth by about 40% as compared to untreated cells. A hit, as defined herein, may also include a candidate agent, compound, or molecule inhibiting growth by about 10% or about 20% or about 30% or about 50% or greater than about 60% as compared to untreated cells.

The terms “include” and “including” are not intended to be limiting in scope.

An “individual” means a vertebrate, preferably a mammal, more preferably a human.

The phrase “loss of function” means an alteration that causes a decrease or the total loss of the activity of the encoded protein. In one aspect, the decrease in activity and/or function is about 30%, or about 40%, or about 50%, or about 60%, or about 70%, or about 80%, or about 90%, or greater than about 95%.

The term “mutation” means an alteration in a DNA or protein sequence, either by site-directed or random mutagenesis. A mutated form of a protein encompasses point mutations as well as insertions, deletions, or rearrangements. A mutant is an organism containing a mutation.

The phrase “NF1-related disorder or condition” means any disease state or disorder or symptoms that result from or is associated with a mutation, deletion, dysregulation or other alteration of the NF1 gene. Such disorders include Neurofibromatosis Type I. Associated conditions include neurofibromas, malignant peripheral nerve sheath tumors, optic gliomas, Schwannomas, gliomas, leukemias, pheochromocytomas and non-tumor manifestations, including learning disorders.

The phrase “non-peptidic compounds” include compounds composed, in whole or in part, of peptidomimetic structures, such as D-amino acids, non-naturally occurring L-amino acids, modified peptide backbones and the like, as well as compounds that are composed, in whole or in part, of molecular structures unrelated to naturally-occurring L-amino acid residues linked by natural peptide bonds. “Non-peptidic compounds” also include natural products.

The term “pharmaceutically-acceptable carrier,” as used herein, means one or more compatible solid or liquid filler diluents or encapsulating substances which are suitable for administration to a mammal

The term “compatible” means that the components of the composition are capable of being commingled with the subject compound, and with each other, in a manner such that there is no interaction which would substantially reduce the pharmaceutical efficacy of the composition under ordinary use situations. When liquid dose forms are used, it may be advantageous for the disclosed compounds to be soluble in the liquid. Pharmaceutically-acceptable carriers must, of course, be of sufficiently high purity and sufficiently low toxicity to render them suitable for administration to the mammal being treated.

The phrase “pharmaceutically acceptable salt(s)” includes salts of acidic or basic groups that may be present in combination with the disclosed compounds. Compounds that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, ptoluenesulfonate and pamoate (i.e., 1,1′-methylene-bis-(2-hydroxy-3-naphthoate)) salts. Compounds that include an amino moiety may form pharmaceutically or cosmetically acceptable salts with various amino acids, in addition to the acids mentioned above. Compounds that are acidic in nature are capable of forming base salts with various pharmacologically or cosmetically acceptable cations. Examples of such salts include alkali metal or alkaline earth metal salts and, particularly, calcium, magnesium, sodium lithium, zinc, potassium, and iron salts.

The term “polypeptide” means any chain of amino acids, regardless of length or post-translational modification (for example, glycosylation or phosphorylation).

The term “potential therapeutic agent” means any candidate compound that may be identified, using the disclosed methods, as having a potential beneficial or therapeutic effect on one or more disorders described herein. Potential therapeutic agents may be identified by their effect on the disclosed, such effect generally comprising inhibition of viability, growth, proliferation, or migration of test cells, although variations of the effect or additional effects that can be measured will be recognized by one of ordinary skill in the art and are included within the scope of the disclosure. Potential therapeutic agents, as used herein, are identified as having a desired effect in vitro, and are considered “hits” which may be subjected to further in vitro or in vivo evaluation to determine or optimize the therapeutic benefit, or, alternatively, may be used to identify derivative or analogous agents which may in turn be evaluated for an in vivo or in vitro therapeutic effect.

The terms “prevent,” “preventing” and “prevention” refer to the prevention of the development, recurrence or onset of a disorder or one or more symptoms thereof resulting from the administration of one or more compounds disclosed herein or the administration of a combination of such a compound and a known therapy for such a disorder.

The terms “prophylactic agent” and “prophylactic agents” refer to any agent(s) which can be used in the prevention of a disorder. In certain embodiments, the term “prophylactic agent” refers to a compound identified in the screening assays described herein. In certain other embodiments, the term “prophylactic agent” refers to an agent other than a compound identified in the screening assays described herein which is known to be useful for, or has been or is currently being used to prevent or impede the onset, development and/or progression of a disorder or one or more symptoms thereof. The term “purified,” in the context of a compound refers to a compound that is substantially free of chemical precursors or other chemicals when chemically synthesized. In a specific embodiment, the compound is 60%, preferably 65%, 70%, 75%, 80%, 85%, 90%, or 99% free of other, different compounds.

The term “small molecule” and analogous terms include peptides, peptidomimetics, amino acids, amino acid analogs, polynucleotides, polynucleotide analogs, nucleotides, nucleotide analogs, organic or inorganic compounds (i.e., including heterorganic and/or ganometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Unless otherwise specifically stated, use of the term “substituted” or “substituent” means any group or atom other than hydrogen. Additionally, when a group, compound or formula containing a substitutable hydrogen is referred to or when the term “group” is used, it means that when a substituent group contains a substitutable hydrogen, it is also intended to encompass not only the substituent's unsubstituted form, but also its form further substituted with any substituent group or groups as herein mentioned, so long as the substituent does not destroy properties necessary for device utility. Suitably, a substituent group may be halogen or may be bonded to the remainder of the molecule by an atom of carbon, silicon, oxygen, nitrogen, phosphorous, sulfur, selenium, or boron. The substituent may be, for example, halogen, such as chloro, bromo or fluoro; nitro; hydroxyl; cyano; carboxyl; or groups which may be further substituted, such as alkyl, including straight or branched chain or cyclic alkyl, such as methyl, trifluoromethyl, ethyl, t-butyl, and cyclohexyl; alkenyl, such as ethylene, 2-butene; alkoxy, such as methoxy, ethoxy, propoxy, butoxy, 2-methoxyethoxy, sec-butoxy, and hexyloxy; aryl such as phenyl, 4-t-butylphenyl, 2,4,6-trimethylphenyl, naphthyl; aryloxy, such as phenoxy, 2-methylphenoxy, alpha- or beta-naphthyloxy, and 4-tolyloxy; carbonamido, such as acetamido, benzamido, butyramido, phenylcarbonylamino, p-tolylcarbonylamino, N-methylureido, NN-dimethylureido, N-phenylureido, and N,N-diphenylureido; sulfonamido, such as methylsulfonamido, benzenesulfonamido, p-tolylsulfonamido, and N,N-dipropyl-sulfamoylamino; sulfamoyl, such as N-methylsulfamoyl, N-ethylsulfamoyl, N,N-dipropylsulfamoyl, and N-phenylsulfamoyl; carbamoyl, such as N-methylcarbamoyl, N,N-dibutylcarbamoyl, N-benzylcarbamoyl; acyl, such as acetyl, propanoyl, benzoyl and 4-methyl benzoyl; oxyacyl, such as phenoxycarbonyl, methoxycarbonyl, butoxycarbonyl, ethoxycarbonyl, and benzyloxycarbonyl; sulfonyl, such as methylsulfonyl, ethylsulfonyl, phenylsulfonyl, 4-fluorophenylsulfonyl, phenoxysulfonyl, and p-tolylsulfonyl; sulfinyl, such as methylsulfinyl, ethylsulfinyl, phenylsulfinyl, and p-tolylsulfinyl; thio, such as methylthio, ethylthio, benzylthio, phenylthio, and p-tolylthio; acyloxy, such as acetyloxy, and benzoyloxy; amine, such as anilino, 2-chloroanilino, dimethylamine, methylamine; a heterocyclic group, a heterocyclic oxy group or a heterocyclic thio group, each of which may be substituted and which contain a 5 to 7 membered heterocyclic ring composed of carbon atoms and at least one hetero atom selected from the group consisting of oxygen, nitrogen, or sulfur, such as 2-furyl, 2-imidazolyl, 4-imidazolyl, 2-thienyl, 2-benzimidazolyloxy or 2-benzothiazolyl; If desired, the substituents may themselves be further substituted one or more times with the described substituent groups. The particular substituents used may be selected by those skilled in the art to attain desirable properties for a specific application and can include, for example, electron-withdrawing groups, electron-donating groups, and steric groups. When a molecule may have two or more substituents, the substituents may be joined together to form a ring such as a fused ring unless otherwise provided. Generally, the above groups and substituents thereof may include those having up to 48 carbon atoms, typically 1 to 36 carbon atoms and usually less than 24 carbon atoms, but greater numbers are possible depending on the particular substituents selected.

The term “therapeutic agent” means any potential therapeutic agent or candidate agent that is determined, using the disclosed methods, to have an in vitro effect on test cells, as described herein, such that the agent would be expected to have, if not demonstrated to have, a beneficial effect on NF1 or NF1-related disorders or conditions. The in vitro effect measured may vary, but generally comprises inhibition of viability, growth, proliferation, or migration of test cells; variations of the effect that can be measured will be recognized by one of ordinary skill in the art. Potential therapeutic agents, as used herein, are identified as having a desired effect in vitro, and are considered “hits” which may be subjected to further in vitro or in vivo evaluation to determine or optimize the therapeutic benefit, or, alternatively, may be used to identify derivative or analogous agents which may in turn be evaluated for an in vitro or in vitro therapeutic effect. Such therapeutic agents are intended to be used in the prevention, treatment, management or amelioration of one or more symptoms of disorders related to NF1 gene mutations, deletions, or dysregulation. The term “therapeutic agent” may refer to a compound, such as a small molecule as defined herein.

The term “therapeutically effective amount” refers to the amount of a therapy (e.g., a therapeutic agent) sufficient to result in (i) the amelioration of one or more symptoms of a disorder, (ii) prevent advancement of a disorder, (iii) cause regression of a disorder, or (iv) to enhance or improve the therapeutic effect(s) of another therapy (e.g., therapeutic agent). The amount of the subject compound is generally sufficient to significantly induce a positive modification in the condition to be treated, but low enough to avoid serious side effects (at a reasonable benefit/risk ratio), within the scope of sound medical judgment. The therapeutically effective amount of the subject compound will vary with the age and physical condition of the patient being treated, the severity of the condition, the duration of the treatment, the nature of concurrent therapy, the particular pharmaceutically-acceptable carrier utilized, and like factors within the knowledge and expertise of the attending physician. Preparing a dosage form is within the purview of the skilled artisan. Examples are provided for the skilled artisan, but are non-limiting, and it is contemplated that the skilled artisan can prepare variations of the compositions claimed.

The terms “therapy” and “therapies” refer to any method, protocol and/or agent that can be used in the prevention, treatment, management or amelioration of a disease or disorder or one or more symptoms thereof.

The terms “treat,” “treatment” and “treating” refer to the reduction or amelioration of the progression, severity and/or duration of a disorder or one or more symptoms thereof.

The term “yeast” means a unicellular fungi. The precise classification is a field that uses the characteristics of the cell, ascospore and colony. Physiological characteristics are also used to identify species. Budding yeasts are true fungi of the phylum Ascomycetes, class Saccharomycetes (also called Hemiascomycetes). The true yeasts are separated into one main order Saccharomycetales. The term “yeast” includes not only yeast in a strictly taxonomic sense, i.e., unicellular organisms, but also yeast-like multicellular fungi or filamentous fungi.

The practice of the disclosed methods will employ, unless otherwise indicated, conventional techniques of molecular biology (including recombinant techniques), microbiology, cell biology and biochemistry, which are within the skill of the art.

It is believed that de-regulation of the protein “Ras” is associated with a wide range of disease states. There are several Ras isoforms in humans. The predominant isoforms believed to be relevant to human cancer are K-Ras (NCBI Accession Number NG 007524, SEQ ID NO: 1) (having two splice variants), H-Ras (NCBI Accession Number NG 007666, SEQ ID NO: 2), and N-Ras (NCBI Accession Number NG 007572; SEQ ID NO: 3). The mammalian R-Ras is most similar to S. cerevisiae Ras1 (SEQ ID: 5) and Ras 2 (SEQ ID: 6). Frequently, tumors acquire mutations in one of these genes that render the protein constitutively active (deregulated). In other disease states, upstream effector molecules may lose function or otherwise be affected such that Ras is deregulated. For example, the Ras signaling pathway may be activated by amplification of certain growth factor receptors, or by activating mutations in growth factor receptor genes. Several other inherited syndromes are associated with deregulated Ras signaling (Ras-opathies), for example NF1, Costello syndrome, Noonan syndrome, and LEOPARD syndrome. These disorders may be caused by deregulation of the Ras signaling pathway, predominantly by activating mutations in K-Ras and H-Ras or loss of upstream regulators.

Similarly, it is believed that the Ras pathway is critical in Candida albicans and other fungal pathogens for the transition from yeast to hyphal forms, such form being believed to be critical for virulence. Candida albicans is a yeast-like fungus that commonly causes infections. Candida albicans lives in the mucous membranes of the mouth, vaginal tract, and the intestines. Certain conditions such as pregnancy, oral contraception, antibiotic use, or a compromised immune system can cause an overgrowth of Candida making it an infection. The three most common areas of Candida infection are the vagina, mouth, and uncircumcised penis. Vaginal Candida infections are commonly called yeast infections, but other fungi can produce a similar vaginal infection. A Candida infection of the mouth is called thrush, and a Candida infection of the uncircumcised penis is called balanitis. These infections can be treated with topical or oral anti-fungals. Accordingly, the assays disclosed herein may be useful for identifying and/or developing novel antifungal therapeutic drugs; novel fungal secondary metabolites; improve yields of presently available fungal products; and develop technologies and products to address unmet fungal challenges. It is believed that the signal transduction machinery is conserved among fungi. Thus, based on the discoveries described herein, each of these signal transduction cascades represents a target for antifungal drugs and/or regulation of secondary metabolites. Strains of S. cerevisiae carrying mutant alleles of any of the genes can be used to screen for fungal homologs, including those from important pathogenic fungi and commercially important fungi, such as Aspergillus sp., Penicillium sp., Acremonium chrysogenum, Yarrowia lipolytica and Phaff a rhodozyma, which are capable of complementing or rescuing the mutant phenotype. These strains can be genetically modified such that the rescued organisms are capable of increased growth or survival, such that these organisms can be isolated using selection based screens described herein. Selection-based screens allow for high-throughput, and thus provide a more rapid approach to gene isolation than those currently used. Moreover, screens for genes which complement mutant phenotypes allows for isolation of genes which share functional properties but which do not contain high degrees of similarity at the nucleotide or amino acid level.

The NF1 protein is a GTPase-activating (“GAP”) protein for Ras proteins. The NF1 gene locus represents a mutational hotspot (14). Loss of NF1 results in increased levels of Ras-GTP (9, 10). NF1 mutation in MPNST cells also leads to increased MAP kinase and PKA activation (38). Loss of function mutations in the Neurofibromatosis type 1 gene (NF1, SEQ ID NO: 7)) results in an autosomal dominant disorder known as Neurofibromatosis type I (NF1) that affects 1 in 2,500 to 3,500 live births. It is believed that activated Ras can lead to many of the phenotypes observed in NF1 patients, such as uncontrolled proliferation and aberrant migration of Schwann cells. 95% of patients will develop neurofibromas that associate with nerve endings (dermal) or large nerves (plexiform). 30% of patients develop plexiform neurofibromas that can cause disfigurement and/or compression of organs, which can have devastating consequences. Furthermore, 8-13% of patients will develop malignant peripheral nerve sheath tumors (“MPNST”s) (4-6), the most severe manifestation of NF1 disease ((4), (5), (6)). These tumors are aggressive soft tissue sarcomas with poor prognosis. Half of all MPNSTs are sporadic in nature; half arise in individuals with loss of function mutations in the NF1 gene. MPNSTs represent a major cause of mortality in NF1 patients.

As traditional treatment using DNA damaging agents frequently leads to secondary malignancies, surgical removal of tumor tissue and the affected nerve is the only treatment, which is often ineffective. Therapeutic options are limited to surgical resection of the neurofibromas and the associated nerve. Excision of the tumor does not always prevent local recurrence, and metastases to the lung, liver, and brain are common. Current therapeutic regimens have limited use because the tumors are generally resistant to standard chemotherapy and radiation. Furthermore, DNA damaging cancer therapies frequently trigger genomic instability, thus when used in young individuals they can induce mutations that will lead to secondary malignancies or malignancies later in life (7, 8). However, in identifying agents that selectively treat or prevent NF1 or NF1 related disorders, the optimal screen would identify compounds that affect migration and/or growth of NF1 mutant but not of wild-type Schwann cells. However, Schwann cells can be difficult to work with and may not be available in large enough quantities to make large scale screening feasible. Thus, alternative screening tools for new compounds are desired.

It is also believed that similar pathways are deregulated and/or dysregulated in many cancers including pancreatic (K-Ras), colon, lung, and other sporadic cancers. Thus, inhibitors and targets identified in this screen could be explored as therapeutic targets for other types of cancer.

In one aspect, a method of identifying novel therapeutic agents for the treatment of a disorder associated with Ras deregulation or dysregulation and/or therapeutic agents that act on or modulate the NF1 pathway, including, for example, associated molecules (such as downstream or upstream signaling or effector molecules) are disclosed. In one aspect, the disorder associated with Ras deregulation or dysregulation may be related to an alteration in the NF1 gene. In one aspect, the disorder may be Neurofibromatosis Type I. In one aspect, the disorder may be a fungal infection, such as a fungal infection associated with or caused by Candida albicans.

In one aspect, a method of identifying a therapeutic agent useful for the treatment of NF1 or an NF1-related disorder or condition is provided, the method comprising the steps of:

a) contacting test cells with a candidate agent;
b) determining viability, proliferation or migration of test cells contacted with the candidate agent; and
c) comparing the viability, proliferation, or migration of the test cells with the viability of an appropriate control group of cells
wherein a candidate agent that effects viability, growth, proliferation or migration is determined to be a potential therapeutic agent for the treatment of an NF1-related disorder or condition.

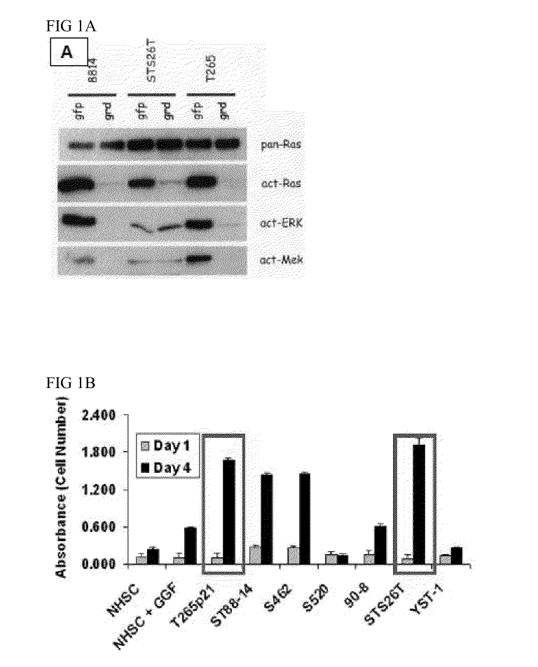

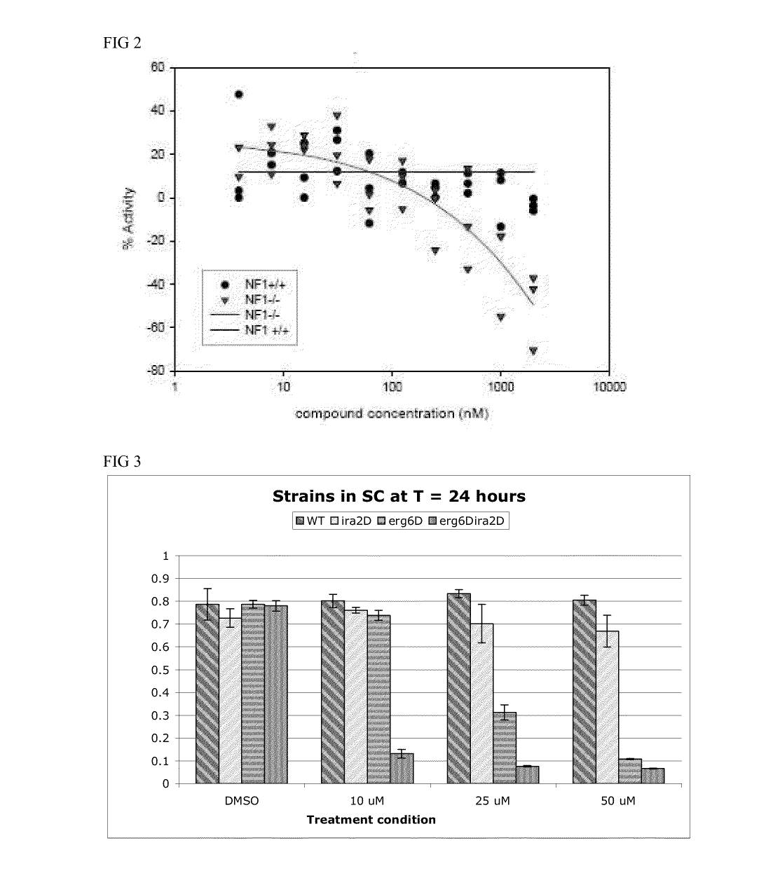

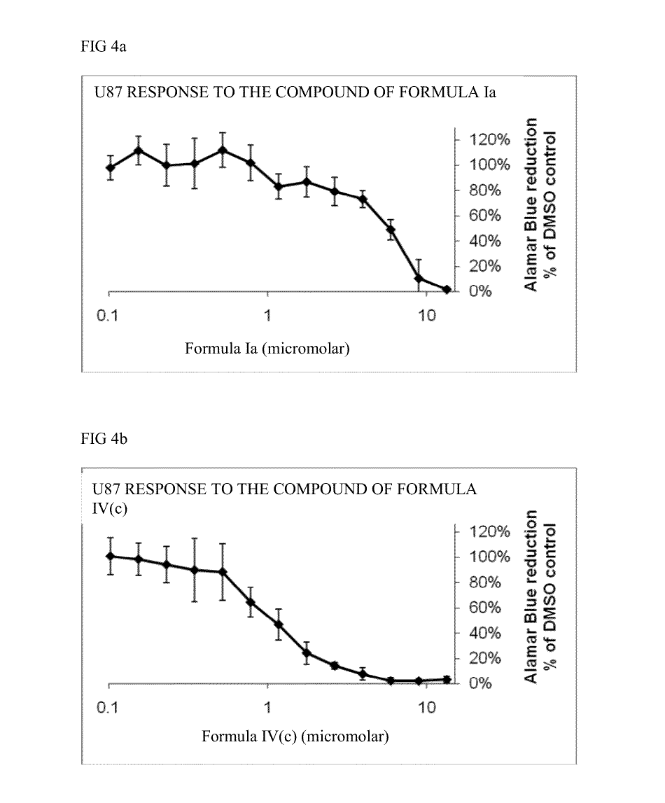

The test cells used in the methods may comprise a population of MPNST cells lacking wild-type NF1 (“NF1−/−”), while the control group may comprise MPNST cells having wild-type NF1 (“NF1+/+”) While these cells have similar gene expression patterns (Miller et al., 2006) they show different basal levels of GTP-bound Ras (Mahller et al., 2006) and phospho-Erk levels. FIG. 1A. This indicates that a molecular signature upstream of Erk exists that differentiates the NF1 status of MPNST cells. In vitro growth, proliferation, cell number or migration of MPNST cells having wild type NF1 (NF1+/+) may then be compared to MPNST cells lacking NF1 (NF1−/−) after exposure to a candidate agent. In this aspect, candidate compounds which effect a change in cell growth, proliferation, number or migration in the NF1−/− cells may be considered a potential therapeutic agent. For determining whether a candidate compound may be a potential therapeutic agent, the effect is generally a decrease in, or inhibition of, cell growth, proliferation, number, or migration.

The disclosed method is based on the observation that re-introduction of the Ras regulatory (GRD) domain of NF1 by adenoviral transduction selectively downregulates ERK phosphorylation in NF1−/− but not NF1+/+ MPNST cells. NF1−/− MPNST cell lines have mutations in both alleles of the NF1 gene, whereas NF1+/+ MPNST cell lines have the wild type NF1 gene on both alleles. FIG. 1B shows ST8814 and T265 NF1 patient MPNST cell lines. STS26T is a non-NF1 patient cell line, with no NF1 mutations identified using DNA analysis (Miller et al., 2006). The three human MPNST cell lines are infected with an NF1 GRD (GRD) adenovirus or a vector encoding green fluorescent protein (vector). Two days later lysates are prepared and blotted directly for total canonical Ras proteins (H,N,K-Ras), active ERK, or active MEK. Ras-GTP was evaluated by centrifuging beads conjugated with GST-Raf-RBD from cell lysates, and probing by Western blots. FIG. 1B demonstrates that cell growth rates vary in the tested MPNST cell lines. 5×103 MPNST cells from each line were seeded in triplicate at equivalent cell numbers on a 24-well plate in their normal growth medium. The same numbers of normal human Schwann cells were plated in the absence (NHSC) or presence of the Schwann cell mitogen glial growth factor (GGF) (NHSC+GGF). Absorbance was normalized to medium-only controls. An 3-(4,5-Dimethylthiazol-2-yl)-2,5-diphenyltetrazolium bromide assay (MTT assay) was performed on day one and day four to assess cell growth and absorbance measured at 540 nm. The GRD causes loss of viability only in MPNST cells that had lost NF1 function. As such, the disclosed screening methods are premised on the finding that signaling events may be deregulated due to a loss of functional NF1, and that molecules that interfere with the deregulated signaling events may be potentially useful therapeutic agents for the disclosed disorders.

Methods of Treating a Disorder Associated with Ras Deregulation or Dysregulation

In one aspect, a method of treating a disorder associated with Ras deregulation or dysregulation is disclosed.

In one aspect, the disorder may be related to an alteration in the NF1 gene. In one aspect, the disorder may be Neurofibromatosis Type I. In one aspect, the disorder may be a fungal infection, such as a fungal infection associated with or caused by Candida albicans.

In one aspect, the disorder may be a proliferative disorder. For example, numerous types of malignant tumors have NF1 mutations. Proliferative disorders that may be treated using the compounds disclosed herein may include glioblastomas, pancreatic cancer; colon cancer; lung cancer; neurofibromas, malignant peripheral nerve sheath tumors, optic gliomas, Schwannomas, gliomas, leukemias, pheochromocytomas, pancreatic adenocarcinoma and combinations thereof. In one aspect, the proliferative disorder may be glioblastoma. Numerous types of malignant tumors are now known to have NF1 mutations. These include neuroblastoma, thyroid tumors and lung cancers. Importantly, 11% of sporadic glioblastomas (GBM) have mutational inactivation of NF1, and others have proteasomal down-regulation of the NF1 protein [4, 5]. Thus many sporadic tumors, including gliomas may also respond to agents targeting NF1 loss. NF1 is mutated in a subset of sporadic GBM and is inactivated by excessive PKC-mediated proteasomal degradation in the absence of NF1 mutations in a subset of human GBM cell lines

In one aspect, the compound of Formula I, for example the compound of Formula Ia, may be used to inhibit the growth of or treat glioblastoma.

In one aspect, the compound of Formula IV, for example, a compound selected from Formula IV(a), Formula IV(b), Formula IV(c), Formula IV(d), or a combination thereof may be used to inhibit the growth of or treat glioblastoma. In one aspect, the compound may be Formula IV(c).

In one aspect, the method may comprise the step of administering a pharmaceutical composition to an individual or organism in need thereof comprising a compound comprising Formula I:

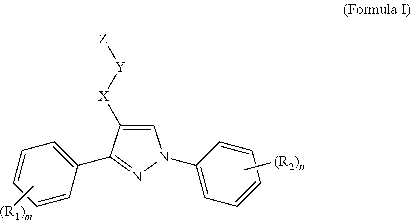

or a pharmaceutically acceptable salt thereof and a pharmaceutically-acceptable carrier; wherein each R₁ and R₂ may be independently selected from halogen; a substituted or unsubstituted aryl group; and a substituted or unsubstituted C₁-C₄ alkyl, C₁-C₄ alkoxy, C₁-C₄ mercapto, and cyano; m and n may be independently an integer from 0 to 5; X—Y may be selected from CH₂—S, CH═N, and CH₂—CH₂; and Z may be selected from amidine, amide, thioamide, hydroxy, and a linear or branched C₁-C₅ alcohol.

In one aspect, each R₁ may be independently selected from —F, —Cl, —Br, phenyl, methoxy, ethoxy, and isopropyloxy; each R₂ may be independently selected from —F, —Cl, —Br, methyl, methoxy, and cyano; m and n may be independently an integer from 0 to 5; X—Y may be selected from CH₂—S and CH═N; and Z may be selected from amidine, amide, hydroxy, and a linear or branched C₁-C₃ alcohol.

In one aspect, R₁ may be a meta-Br; m may be 1; n may be 0; X—Y may be —CH₂—S—; and Z may be an amidine.

In one aspect, the compound may comprise the structure:

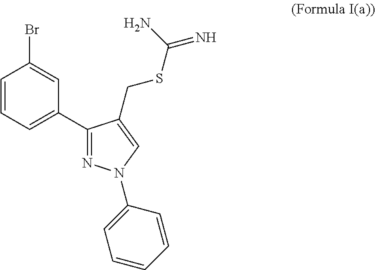 [SMILES: N=C(N)SCc1cn(-c2ccccc2)nc1-c1cccc(Br)c1]

In one aspect, the method may comprise the step of administering a pharmaceutical composition to an individual or organism in need thereof comprising a compound comprising Formula II:

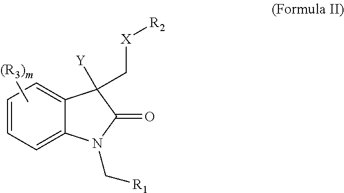

or a pharmaceutically acceptable salt thereof, and a pharmaceutically-acceptable carrier wherein X may be selected from C═O, CHOH, and CH₂; Y may be selected from hydroxy, methyl, alkoxy, amine, and alkyl amine; R₁ may be selected from a substituted or unsubstituted phenyl group, ethenyl, and ethynyl, wherein the phenyl substituent is one or more groups independently selected from F, Cl, Br, methyl, ethyl, hydroxy, methoxy, nitro, and cyano; R₂ may be selected from

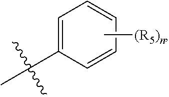

heteroaryl, or naphthyl groups; each R₃ may be independently selected from —F, —Cl, —Br, methyl, ethyl, hydroxy, and methoxy; each R₅ may be independently selected from —F, —Cl, —Br, methyl, ethyl, hydroxy, methoxy, nitro, cyano, and a 5-6 member fused heterocycle containing 1-2 oxygen or nitrogen atoms where the fused heterocycle is formed from two adjacent R₅ groups; m may be an integer from 0 to 4; and n may be an integer from 0 to 5.

In one aspect, X may be selected from C═O and CHOH; Y is selected from hydroxy, methyl, methoxy; R₁ is selected from a substituted or unsubstituted phenyl group, ethenyl, and ethynyl, wherein the phenyl substituent may be one or more groups independently selected from —F, —Cl, —Br, methyl, ethyl, hydroxy, methoxy, nitro, and cyano; R₂ is selected from

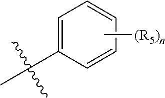

and 4-pyridyl; each R₃ is independently selected from —F, —Cl, —Br, methyl, ethyl, hydroxy, and methoxy; each R₅ may be independently selected from —F, —Cl, —Br, methyl, ethyl, hydroxy, methoxy, nitro, and cyano; and m may be an integer from 0 to 4; and n may be an integer from 0 to 5.

In one aspect, X may be C═O; Y is hydroxy; R₁ may be selected from phenyl, meta-toluene, ethenyl, and ethynyl; and R₂ i may be selected from phenyl and 4-pyridyl; and m may be 0.

In one aspect, the compound may comprise a compound having a structure selected from

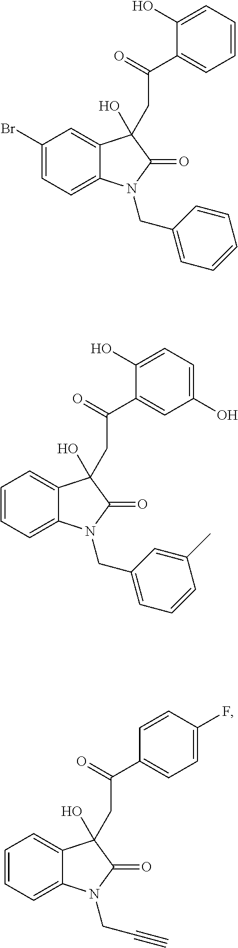 [SMILES: O=C(CC1(O)C(=O)N(Cc2ccccc2)c2ccc(Br)cc21)c1ccccc1O ; Cc1cccc(CN2C(=O)C(O)(CC(=O)c3cc(O)ccc3O)c3ccccc32)c1 ; C#CCN1C(=O)C(O)(CC(=O)c2ccc(F)cc2)c2ccccc21]

and combinations thereof.

In one aspect, the method may comprise the step of administering a pharmaceutical composition to an individual or organism in need thereof comprising a compound comprising Formula III:

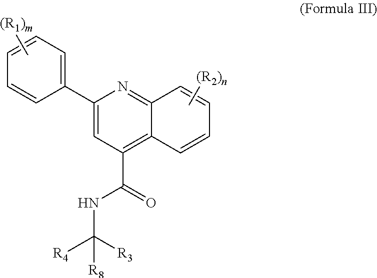

or a pharmaceutically acceptable salt thereof, and a pharmaceutically-acceptable carrier; wherein each R₁ may be independently selected from hydroxy; C₁-C₆ alkyl; C₁-C₆ alkoxy; amine; C₁-C₆ alkyl amino; a fused ring of formula —O(CH₂)ₖO— formed from two adjacent R₁ groups where k may be 1 or 2; and a fused ring of formula —N(CH₂)ₚX— formed from two adjacent R₁ groups where p may be 1 or 2, and X is O, N, or S; each R₂ may be independently selected from C₁-C₄ alkyl and C₁-C₄ alkoxy; R₃ may be selected from hydrogen, methyl, ethyl, propyl, isopropyl, isobutyl, phenyl, phenylmethyl, —CH₂OCH₂Ph, —CH₂SCH₂Ph, —CH₂SCH₂NHCOMe, CH₂CH₂SMe, para-hydroxy phenyl, para-benzyloxy phenyl, —(CH₂)_{q}NHCO₂Ph where q may be an integer from 1 to 4, and —(CH₂)_{w}CO₂cHex where w may be 1 or 2; R₄ may be selected from

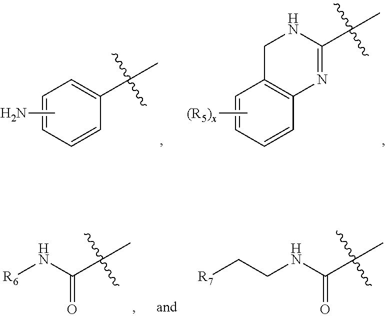

where y is 1 or 2; each R₅ may be independently selected from C₁-C₄ alkyl, C₁-C₄ alkoxy, halogen, and cyano; x is an integer from 0 to 4; R₆ i may be selected from —CH₂CMe₂CH₂NMe₂, —CHMeCH₂CH₂CH₂NEt₂, and

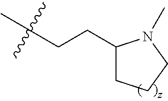

where z is 1 or 2; R₇ may be selected from —NMe₂, —NEt₂, —NiPr_{z}, —NPr₂, 1-pyrrolidine, 1-piperidine, and 4-methylpiperazine; R₈ may be selected from hydrogen and methyl; m may be an integer from 0 to 5; and n may be an integer from 0 to 4.

In one aspect, each R₁ is independently selected from hydroxy; methyl; C₁-C₄ alkoxy; and a fused ring of formula —O(CH₂)ₖO— formed from two adjacent R₁ groups where k is 1 or 2; each R₂ is independently selected from C₁-C₄ alkyl and C₁-C₄ alkoxy; R₃ is selected from hydrogen, methyl, ethyl, propyl, isopropyl, isobutyl, phenyl, phenylmethyl, —CH₂OCH₂Ph, —CH₂SCH₂Ph, —CH₂SCH₂NHCOMe, CH₂CH₂SMe, para-hydroxy phenyl, para-benzyloxy phenyl, —(CH₂)_{q}NHCO₂Ph, where q is an integer from 1 to 4, and —(CH₂)_{w}CO₂cHex, where w is 1 or 2; R₄ is selected from

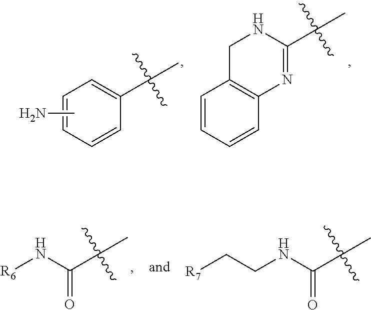

wherein y may be 1 or 2; R₆ is selected from —CH₂CMe₂CH₂NMe₂, —CHMeCH₂CH₂CH₂NEt₂, and

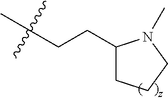

wherein z may be 1 or 2; R₇ may be selected from —NMe₂, —NEt₂, —NiPr₂, —NPr₂, 1-pyrrolidine, 1-piperidine, and 4-methylpiperazine; R₈ may be selected from hydrogen and methyl; m may be an integer from 0 to 5; and n may be an integer from 0 to 4.

In one aspect, the compound may comprise a compound selected from

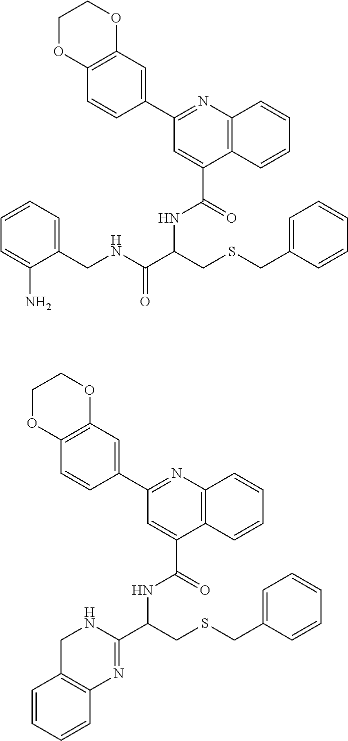 [SMILES: Nc1ccccc1CNC(=O)C(CSCc1ccccc1)NC(=O)c1cc(-c2ccc3c(c2)OCCO3)nc2ccccc12 ; O=C(NC(CSCc1ccccc1)C1=Nc2ccccc2CN1)c1cc(-c2ccc3c(c2)OCCO3)nc2ccccc12]

or combinations thereof.

In one aspect, the compound may comprise a compound having a structure selected from Table 1

TABLE 1

| Compound # | Structure |
|---|---|
| 1 | 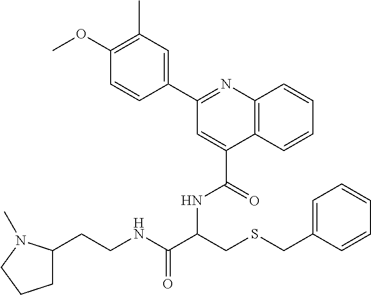 [SMILES: COc1ccc(-c2cc(C(=O)NC(CSCc3ccccc3)C(=O)NCCC3CCCN3C)c3ccccc3n2)cc1C] |
| 2 | 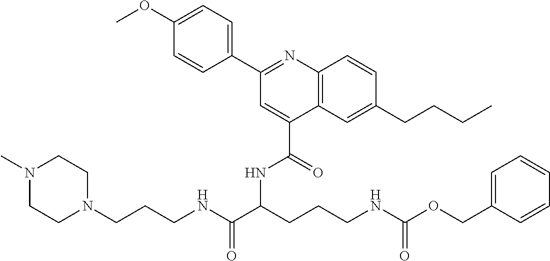 [SMILES: CCCCc1ccc2nc(-c3ccc(OC)cc3)cc(C(=O)NC(CCCNC(=O)OCc3ccccc3)C(=O)NCCCN3CCN(C)CC3)c2c1] |
| 3 | 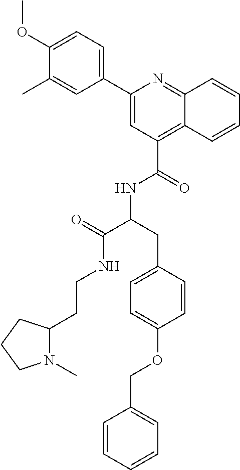 [SMILES: COc1ccc(-c2cc(C(=O)NC(Cc3ccc(OCc4ccccc4)cc3)C(=O)NCCC3CCCN3C)c3ccccc3n2)cc1C] |
| 4 | 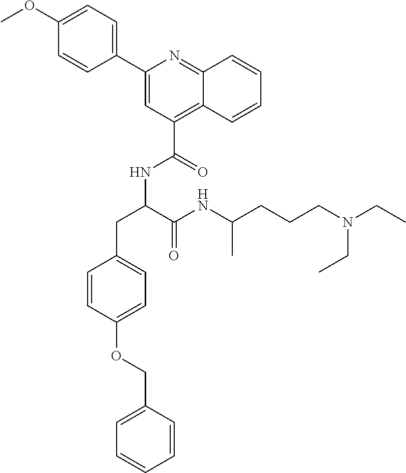 [SMILES: CCN(CC)CCCC(C)NC(=O)C(Cc1ccc(OCc2ccccc2)cc1)NC(=O)c1cc(-c2ccc(OC)cc2)nc2ccccc12] |
| 5 | 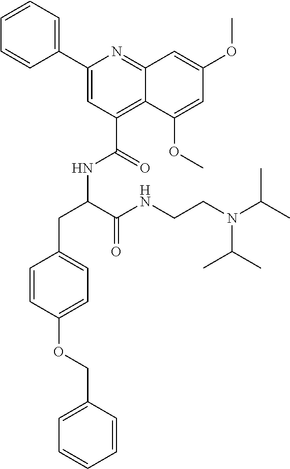 [SMILES: COc1cc(OC)c2c(C(=O)NC(Cc3ccc(OCc4ccccc4)cc3)C(=O)NCCN(C(C)C)C(C)C)cc(-c3ccccc3)nc2c1] |
| 6 | 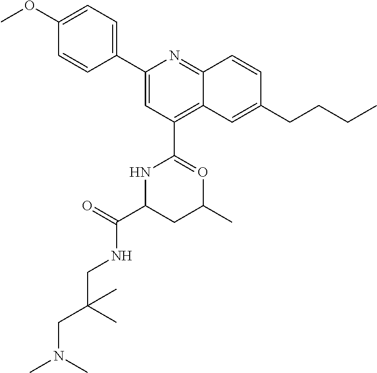 |
| 7 | 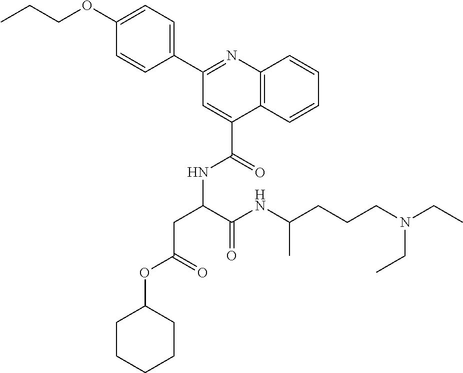 [SMILES: CCCOc1ccc(-c2cc(C(=O)NC(CC(=O)OC3CCCCC3)C(=O)NC(C)CCCN(CC)CC)c3ccccc3n2)cc1] |
| 8 | 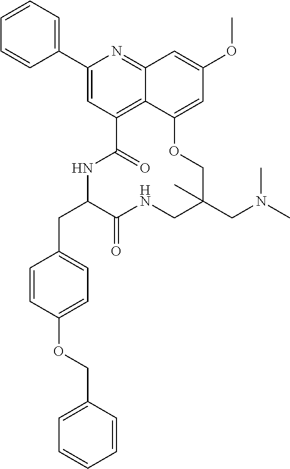 [SMILES: COc1cc2c3c(cc(-c4ccccc4)nc3c1)C(=O)NC(Cc1ccc(OCc3ccccc3)cc1)C(=O)NCC(C)(CN(C)C)CO2] |
| 9 | 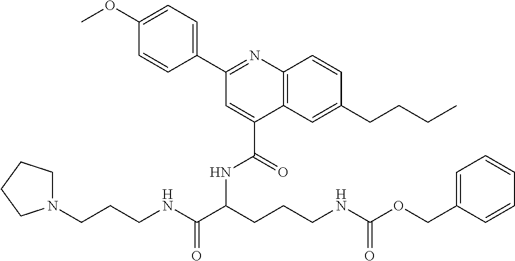 [SMILES: CCCCc1ccc2nc(-c3ccc(OC)cc3)cc(C(=O)NC(CCCNC(=O)OCc3ccccc3)C(=O)NCCCN3CCCC3)c2c1] |
| 10 | 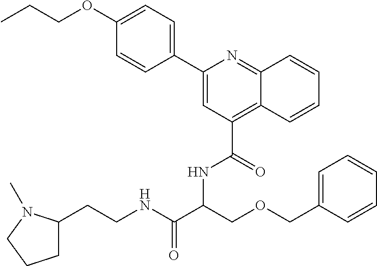 [SMILES: CCCOc1ccc(-c2cc(C(=O)NC(COCc3ccccc3)C(=O)NCCC3CCCN3C)c3ccccc3n2)cc1] |
| 11 | 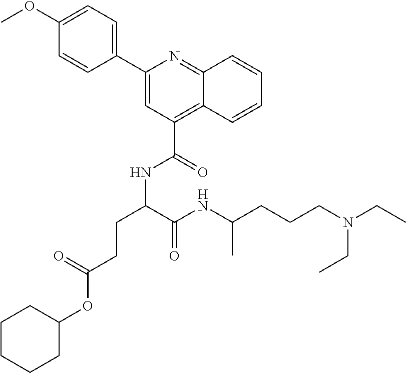 [SMILES: CCN(CC)CCCC(C)NC(=O)C(CCC(=O)OC1CCCCC1)NC(=O)c1cc(-c2ccc(OC)cc2)nc2ccccc12] |
| 12 | 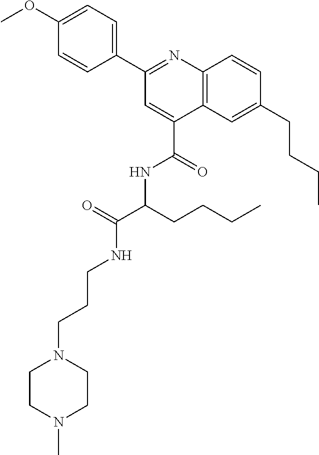 [SMILES: CCCCc1ccc2nc(-c3ccc(OC)cc3)cc(C(=O)NC(CCCC)C(=O)NCCCN3CCN(C)CC3)c2c1] |
| 13 | 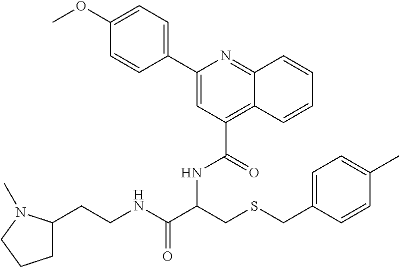 [SMILES: COc1ccc(-c2cc(C(=O)NC(CSCc3ccc(C)cc3)C(=O)NCCC3CCCN3C)c3ccccc3n2)cc1] |
| 14 | 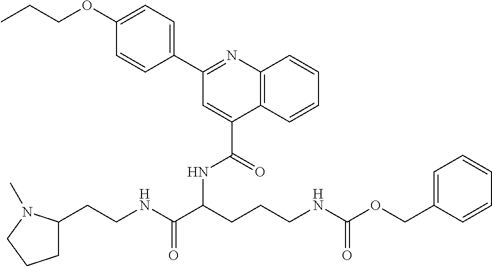 [SMILES: CCCOc1ccc(-c2cc(C(=O)NC(CCCNC(=O)OCc3ccccc3)C(=O)NCCC3CCCN3C)c3ccccc3n2)cc1] |
| 15 | 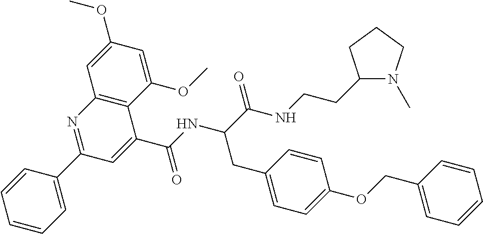 [SMILES: COc1cc(OC)c2c(C(=O)NC(Cc3ccc(OCc4ccccc4)cc3)C(=O)NCCC3CCCN3C)cc(-c3ccccc3)nc2c1] |
| 16 | 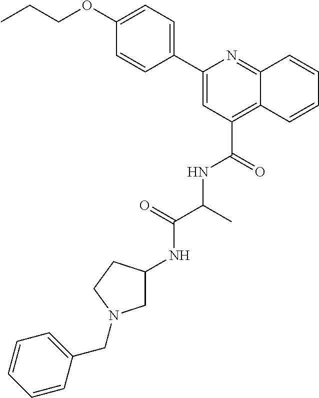 [SMILES: CCCOc1ccc(-c2cc(C(=O)NC(C)C(=O)NC3CCN(Cc4ccccc4)C3)c3ccccc3n2)cc1] |
| 17 | 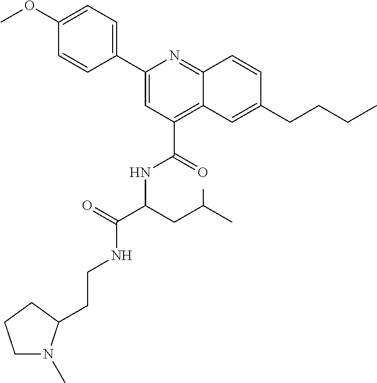 [SMILES: CCCCc1ccc2nc(-c3ccc(OC)cc3)cc(C(=O)NC(CC(C)C)C(=O)NCCC3CCCN3C)c2c1] |
| 18 | 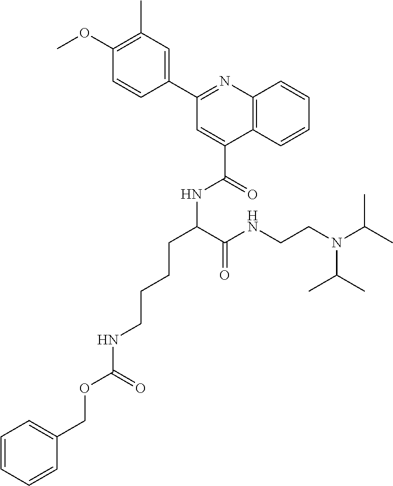 [SMILES: COc1ccc(-c2cc(C(=O)NC(CCCCNC(=O)OCc3ccccc3)C(=O)NCCN(C(C)C)C(C)C)c3ccccc3n2)cc1C] |
| 19 | 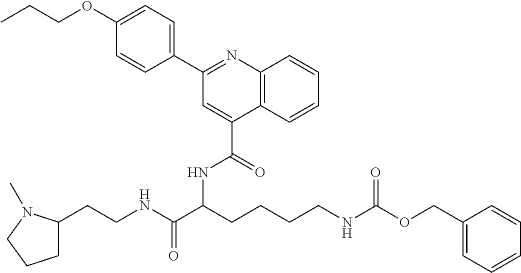 [SMILES: CCCOc1ccc(-c2cc(C(=O)NC(CCCCNC(=O)OCc3ccccc3)C(=O)NCCC3CCCN3C)c3ccccc3n2)cc1] |
| 20 | 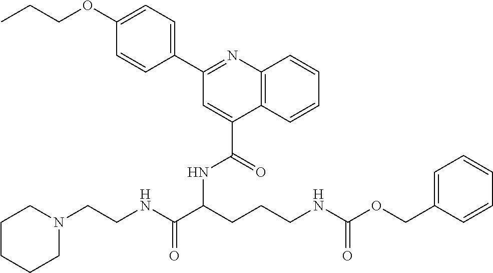 [SMILES: CCCOc1ccc(-c2cc(C(=O)NC(CCCNC(=O)OCc3ccccc3)C(=O)NCCN3CCCCC3)c3ccccc3n2)cc1] |
| 21 | 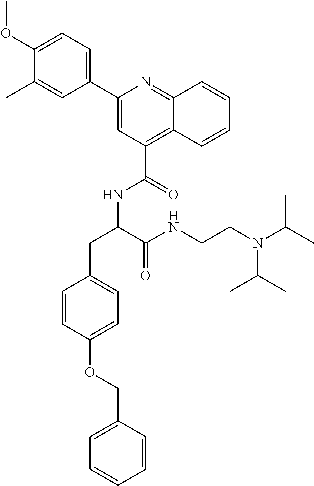 [SMILES: COc1ccc(-c2cc(C(=O)NC(Cc3ccc(OCc4ccccc4)cc3)C(=O)NCCN(C(C)C)C(C)C)c3ccccc3n2)cc1C] |
| 22 | 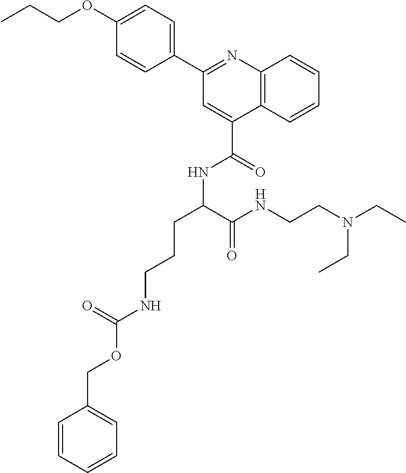 [SMILES: CCCOc1ccc(-c2cc(C(=O)NC(CCCNC(=O)OCc3ccccc3)C(=O)NCCN(CC)CC)c3ccccc3n2)cc1] |
| 23 | 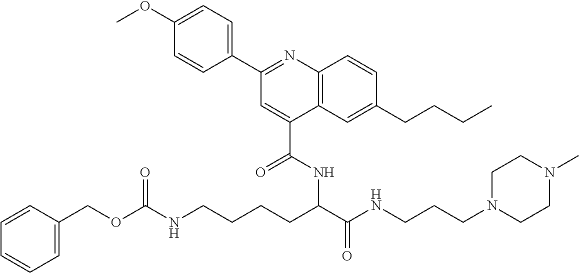 [SMILES: CCCCc1ccc2nc(-c3ccc(OC)cc3)cc(C(=O)NC(CCCCNC(=O)OCc3ccccc3)C(=O)NCCCN3CCN(C)CC3)c2c1] |
| 24 | 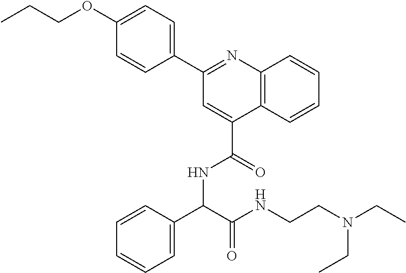 [SMILES: CCCOc1ccc(-c2cc(C(=O)NC(C(=O)NCCN(CC)CC)c3ccccc3)c3ccccc3n2)cc1] |
| 25 | 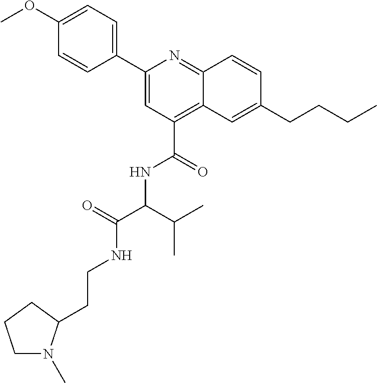 [SMILES: CCCCc1ccc2nc(-c3ccc(OC)cc3)cc(C(=O)NC(C(=O)NCCC3CCCN3C)C(C)C)c2c1] |
| 26 | 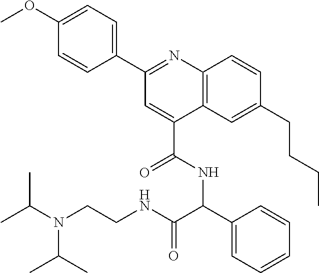 [SMILES: CCCCc1ccc2nc(-c3ccc(OC)cc3)cc(C(=O)NC(C(=O)NCCN(C(C)C)C(C)C)c3ccccc3)c2c1] |
| 27 | 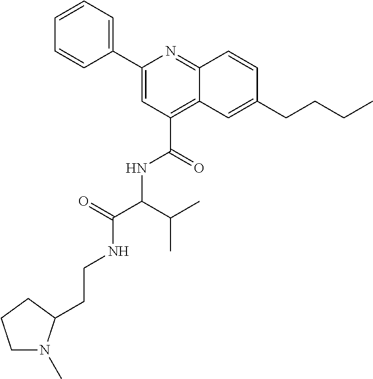 [SMILES: CCCCc1ccc2nc(-c3ccccc3)cc(C(=O)NC(C(=O)NCCC3CCCN3C)C(C)C)c2c1] |
| 28 | 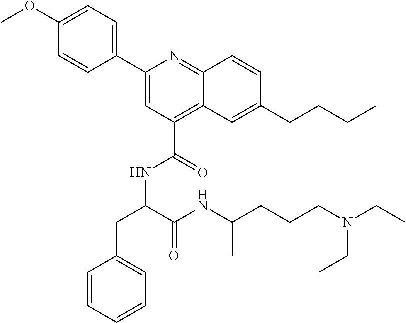 [SMILES: CCCCc1ccc2nc(-c3ccc(OC)cc3)cc(C(=O)NC(Cc3ccccc3)C(=O)NC(C)CCCN(CC)CC)c2c1] |
| 29 | 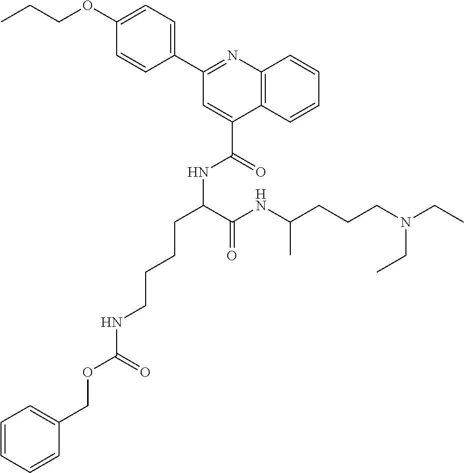 [SMILES: CCCOc1ccc(-c2cc(C(=O)NC(CCCCNC(=O)OCc3ccccc3)C(=O)NC(C)CCCN(CC)CC)c3ccccc3n2)cc1] |
| 30 | 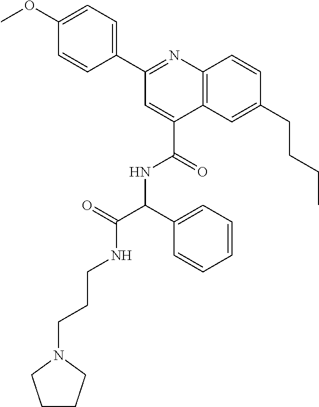 [SMILES: CCCCc1ccc2nc(-c3ccc(OC)cc3)cc(C(=O)NC(C(=O)NCCCN3CCCC3)c3ccccc3)c2c1] |
| 31 | 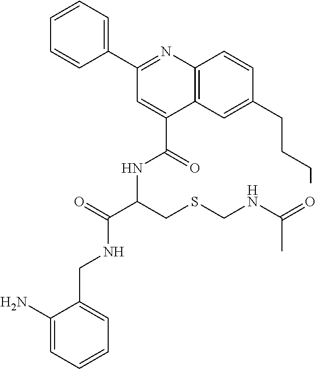 [SMILES: CCCCc1ccc2nc(-c3ccccc3)cc(C(=O)NC(CSCNC(C)=O)C(=O)NCc3ccccc3N)c2c1] |
| 32 | 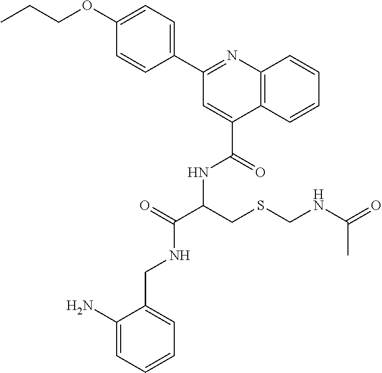 [SMILES: CCCOc1ccc(-c2cc(C(=O)NC(CSCNC(C)=O)C(=O)NCc3ccccc3N)c3ccccc3n2)cc1] |
| 33 | 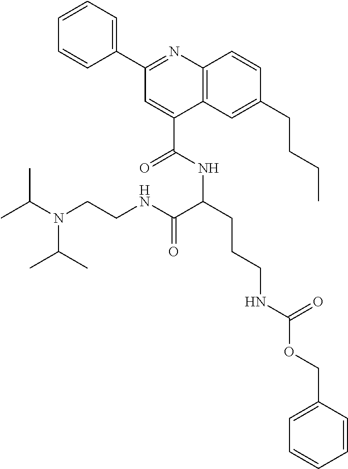 [SMILES: CCCCc1ccc2nc(-c3ccccc3)cc(C(=O)NC(CCCNC(=O)OCc3ccccc3)C(=O)NCCN(C(C)C)C(C)C)c2c1] |
| 34 | 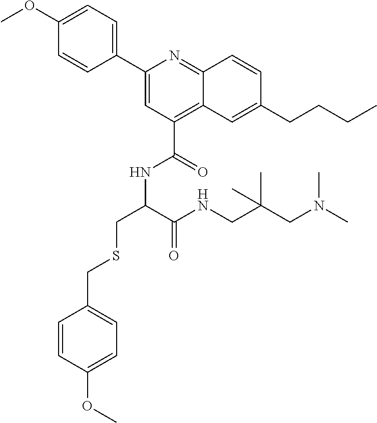 [SMILES: CCCCc1ccc2nc(-c3ccc(OC)cc3)cc(C(=O)NC(CSCc3ccc(OC)cc3)C(=O)NCC(C)(C)CN(C)C)c2c1] |
| 35 | 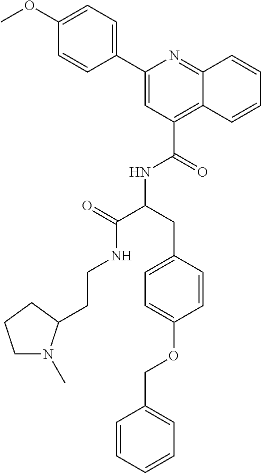 [SMILES: COc1ccc(-c2cc(C(=O)NC(Cc3ccc(OCc4ccccc4)cc3)C(=O)NCCC3CCCN3C)c3ccccc3n2)cc1] |
| 36 | 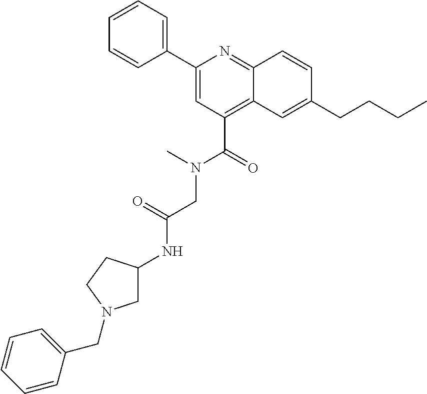 [SMILES: CCCCc1ccc2nc(-c3ccccc3)cc(C(=O)N(C)CC(=O)NC3CCN(Cc4ccccc4)C3)c2c1] |
| 37 | 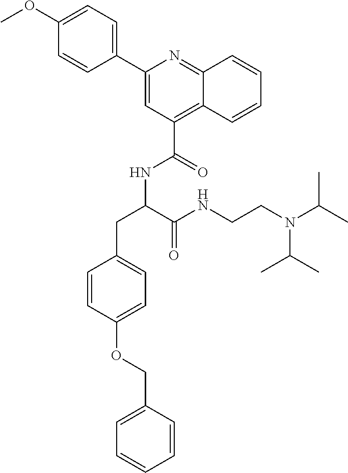 [SMILES: COc1ccc(-c2cc(C(=O)NC(Cc3ccc(OCc4ccccc4)cc3)C(=O)NCCN(C(C)C)C(C)C)c3ccccc3n2)cc1] |
| 38 | 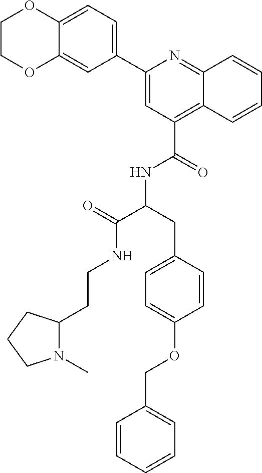 [SMILES: CN1CCCC1CCNC(=O)C(Cc1ccc(OCc2ccccc2)cc1)NC(=O)c1cc(-c2ccc3c(c2)OCCO3)nc2ccccc12] |
| 39 | 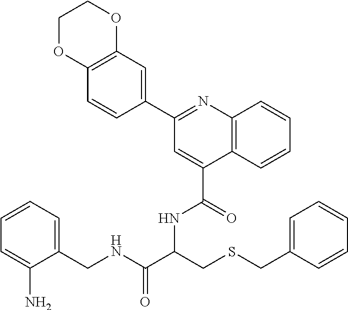 [SMILES: Nc1ccccc1CNC(=O)C(CSCc1ccccc1)NC(=O)c1cc(-c2ccc3c(c2)OCCO3)nc2ccccc12] |
| 40 | 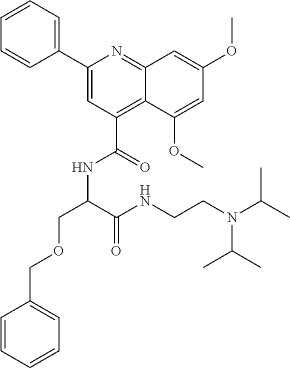 [SMILES: COc1cc(OC)c2c(C(=O)NC(COCc3ccccc3)C(=O)NCCN(C(C)C)C(C)C)cc(-c3ccccc3)nc2c1] |
| 41 | 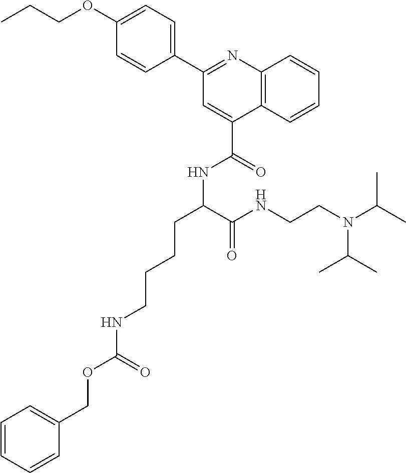 [SMILES: CCCOc1ccc(-c2cc(C(=O)NC(CCCCNC(=O)OCc3ccccc3)C(=O)NCCN(C(C)C)C(C)C)c3ccccc3n2)cc1] |
| 42 | 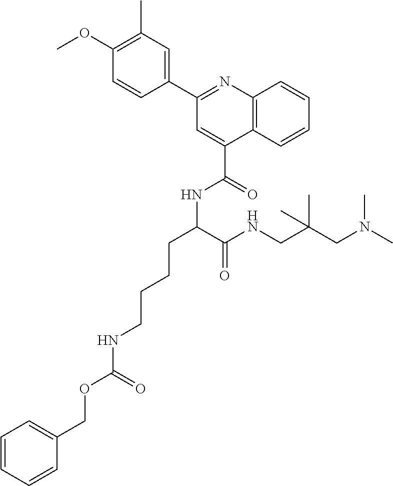 [SMILES: COc1ccc(-c2cc(C(=O)NC(CCCCNC(=O)OCc3ccccc3)C(=O)NCC(C)(C)CN(C)C)c3ccccc3n2)cc1C] |
| 43 | 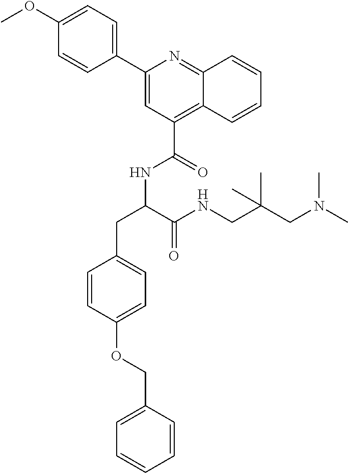 |
| 44 | 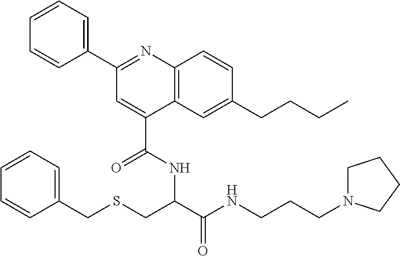 [SMILES: CCCCc1ccc2nc(-c3ccccc3)cc(C(=O)NC(CSCc3ccccc3)C(=O)NCCCN3CCCC3)c2c1] |
| 45 | 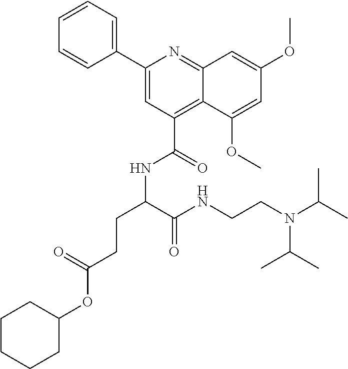 [SMILES: COc1cc(OC)c2c(C(=O)NC(CCC(=O)OC3CCCCC3)C(=O)NCCN(C(C)C)C(C)C)cc(-c3ccccc3)nc2c1] |
| 46 | 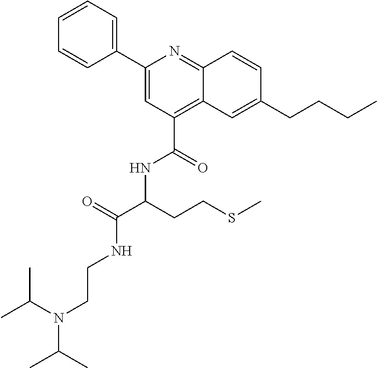 [SMILES: CCCCc1ccc2nc(-c3ccccc3)cc(C(=O)NC(CCSC)C(=O)NCCN(C(C)C)C(C)C)c2c1] |
| 47 | 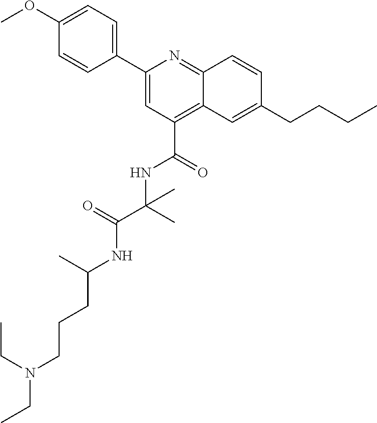 [SMILES: CCCCc1ccc2nc(-c3ccc(OC)cc3)cc(C(=O)NC(C)(C)C(=O)NC(C)CCCN(CC)CC)c2c1] |
| 48 | 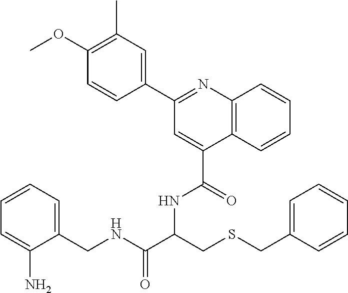 [SMILES: COc1ccc(-c2cc(C(=O)NC(CSCc3ccccc3)C(=O)NCc3ccccc3N)c3ccccc3n2)cc1C] |
| 49 | 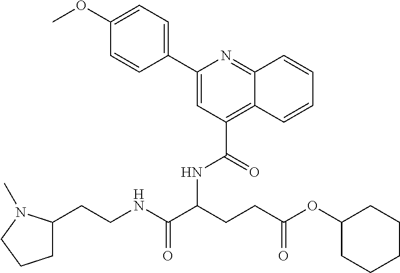 [SMILES: COc1ccc(-c2cc(C(=O)NC(CCC(=O)OC3CCCCC3)C(=O)NCCC3CCCN3C)c3ccccc3n2)cc1] |
| 50 | 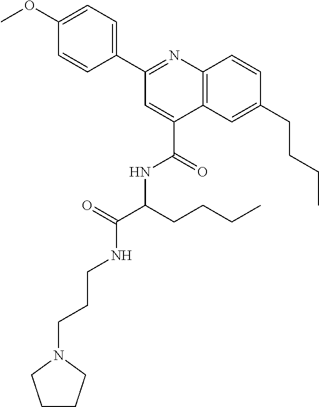 [SMILES: CCCCc1ccc2nc(-c3ccc(OC)cc3)cc(C(=O)NC(CCCC)C(=O)NCCCN3CCCC3)c2c1] |
| 51 | 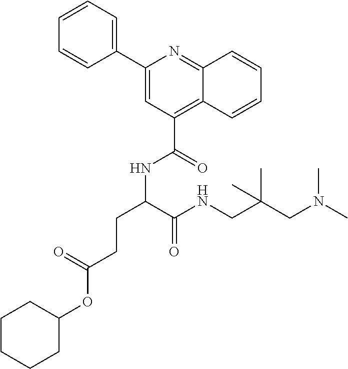 [SMILES: CN(C)CC(C)(C)CNC(=O)C(CCC(=O)OC1CCCCC1)NC(=O)c1cc(-c2ccccc2)nc2ccccc12] |
| 52 | 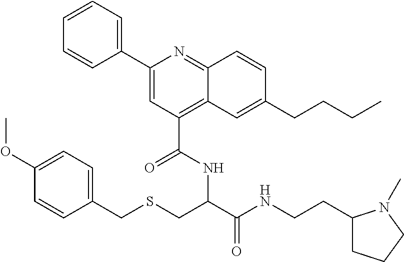 [SMILES: CCCCc1ccc2nc(-c3ccccc3)cc(C(=O)NC(CSCc3ccc(OC)cc3)C(=O)NCCC3CCCN3C)c2c1] |
| 53 | 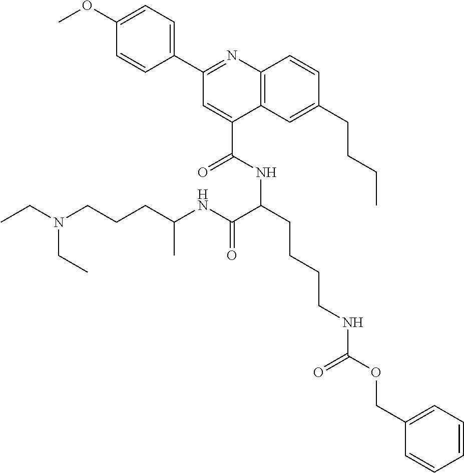 [SMILES: CCCCc1ccc2nc(-c3ccc(OC)cc3)cc(C(=O)NC(CCCCNC(=O)OCc3ccccc3)C(=O)NC(C)CCCN(CC)CC)c2c1] |
| 54 | 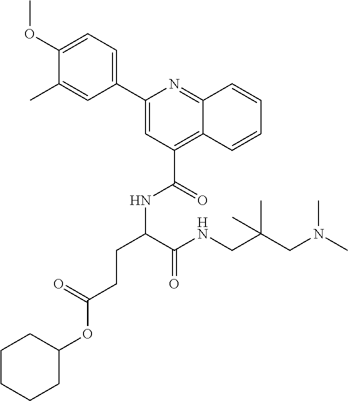 [SMILES: COc1ccc(-c2cc(C(=O)NC(CCC(=O)OC3CCCCC3)C(=O)NCC(C)(C)CN(C)C)c3ccccc3n2)cc1C] |
| 55 | 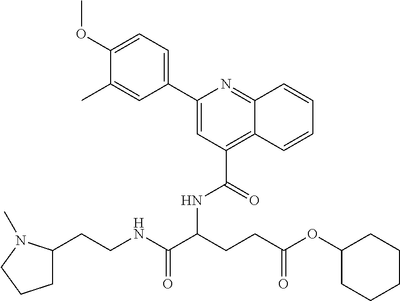 [SMILES: COc1ccc(-c2cc(C(=O)NC(CCC(=O)OC3CCCCC3)C(=O)NCCC3CCCN3C)c3ccccc3n2)cc1C] |
| 56 | 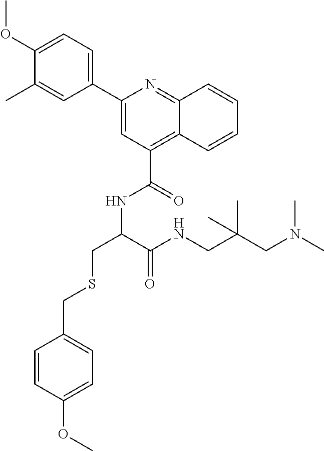 |
| 57 | 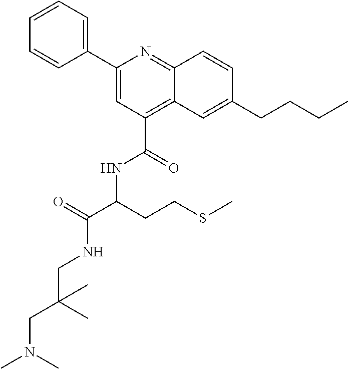 [SMILES: CCCCc1ccc2nc(-c3ccccc3)cc(C(=O)NC(CCSC)C(=O)NCC(C)(C)CN(C)C)c2c1] |
| 58 | 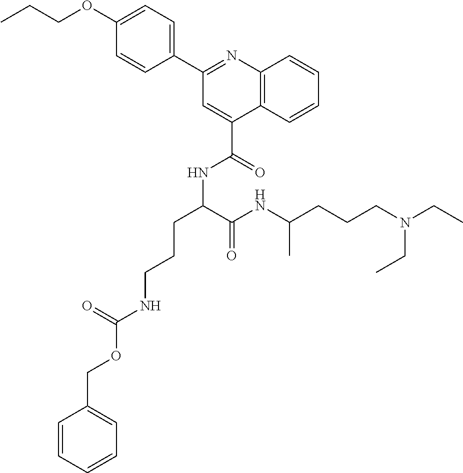 [SMILES: CCCOc1ccc(-c2cc(C(=O)NC(CCCNC(=O)OCc3ccccc3)C(=O)NC(C)CCCN(CC)CC)c3ccccc3n2)cc1] |
| 59 | 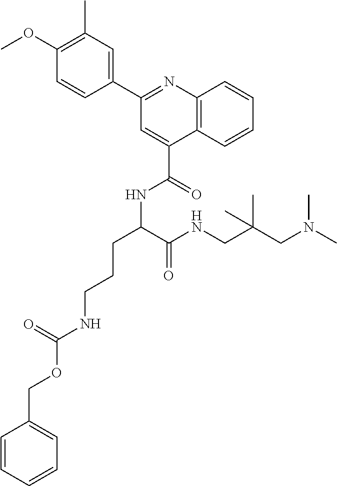 [SMILES: COc1ccc(-c2cc(C(=O)NC(CCCNC(=O)OCc3ccccc3)C(=O)NCC(C)(C)CN(C)C)c3ccccc3n2)cc1C] |
| 60 | 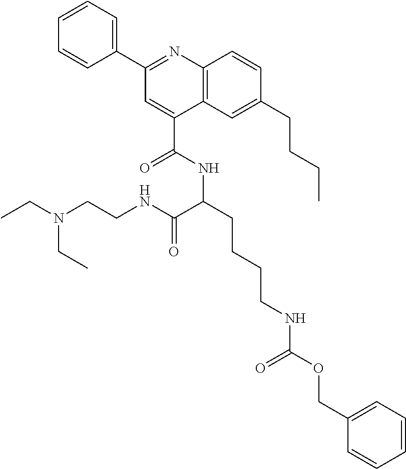 [SMILES: CCCCc1ccc2nc(-c3ccccc3)cc(C(=O)NC(CCCCNC(=O)OCc3ccccc3)C(=O)NCCN(CC)CC)c2c1] |
| 61 | 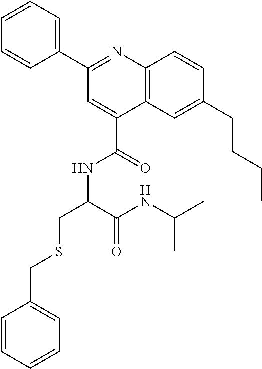 [SMILES: CCCCc1ccc2nc(-c3ccccc3)cc(C(=O)NC(CSCc3ccccc3)C(=O)NC(C)C)c2c1] |
| 62 | 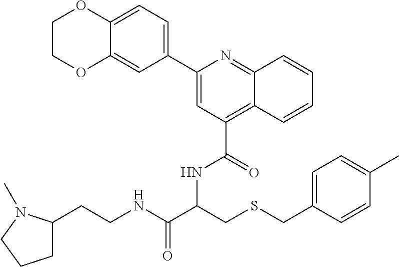 [SMILES: Cc1ccc(CSCC(NC(=O)c2cc(-c3ccc4c(c3)OCCO4)nc3ccccc23)C(=O)NCCC2CCCN2C)cc1] |
| 63 | 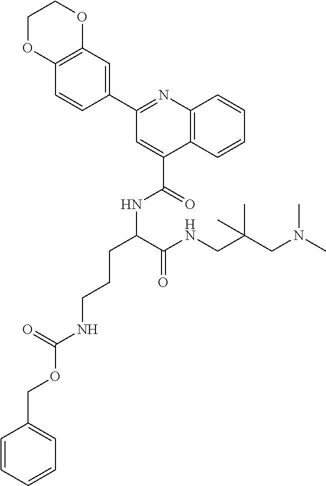 [SMILES: CN(C)CC(C)(C)CNC(=O)C(CCCNC(=O)OCc1ccccc1)NC(=O)c1cc(-c2ccc3c(c2)OCCO3)nc2ccccc12] |
| 64 | 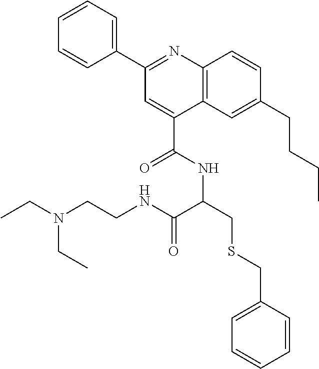 [SMILES: CCCCc1ccc2nc(-c3ccccc3)cc(C(=O)NC(CSCc3ccccc3)C(=O)NCCN(CC)CC)c2c1] |
| 65 | 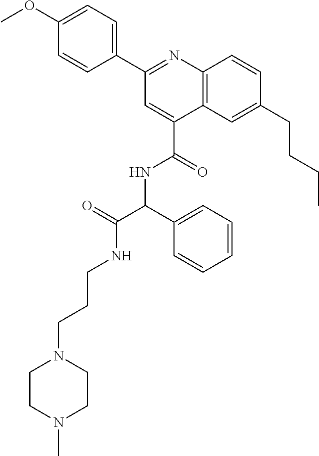 [SMILES: CCCCc1ccc2nc(-c3ccc(OC)cc3)cc(C(=O)NC(C(=O)NCCCN3CCN(C)CC3)c3ccccc3)c2c1] |
| 66 | 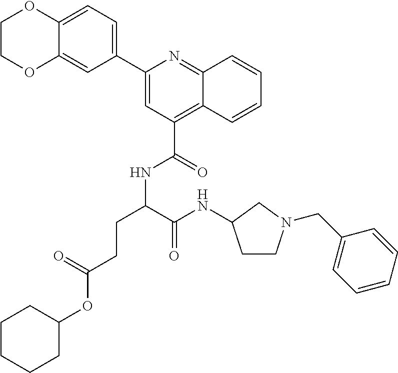 [SMILES: O=C(CCC(NC(=O)c1cc(-c2ccc3c(c2)OCCO3)nc2ccccc12)C(=O)NC1CCN(Cc2ccccc2)C1)OC1CCCCC1] |
| 67 | 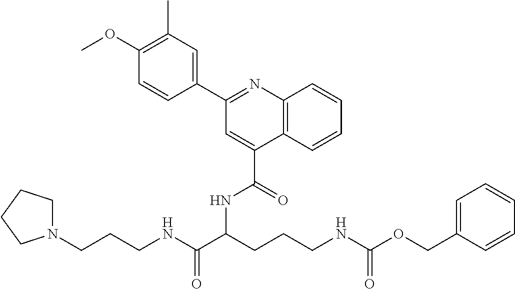 [SMILES: COc1ccc(-c2cc(C(=O)NC(CCCNC(=O)OCc3ccccc3)C(=O)NCCCN3CCCC3)c3ccccc3n2)cc1C] |
| 68 | 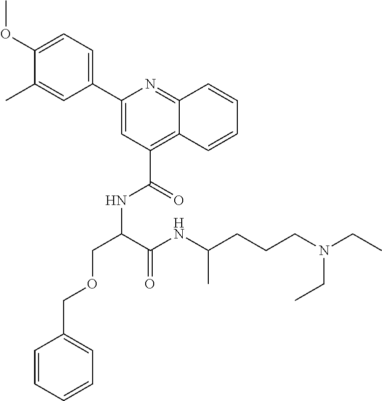 [SMILES: CCN(CC)CCCC(C)NC(=O)C(COCc1ccccc1)NC(=O)c1cc(-c2ccc(OC)c(C)c2)nc2ccccc12] |
| 69 | 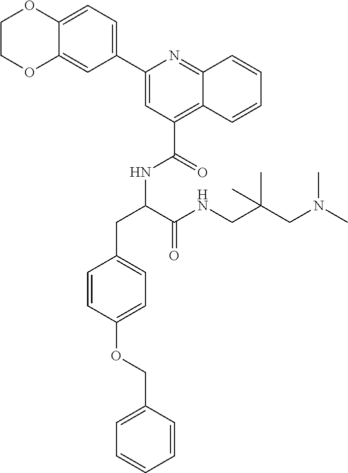 [SMILES: CN(C)CC(C)(C)CNC(=O)C(Cc1ccc(OCc2ccccc2)cc1)NC(=O)c1cc(-c2ccc3c(c2)OCCO3)nc2ccccc12] |
| 70 | 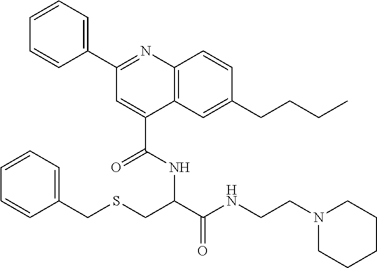 [SMILES: CCCCc1ccc2nc(-c3ccccc3)cc(C(=O)NC(CSCc3ccccc3)C(=O)NCCN3CCCCC3)c2c1] |

In one aspect, the method may comprise the step of administering a pharmaceutical composition to an individual or organism in need thereof comprising a compound comprising Formula IV

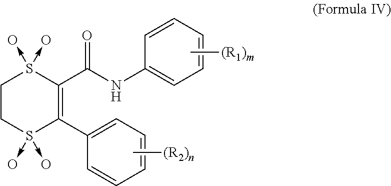

or a pharmaceutically acceptable salt thereof and a pharmaceutically-acceptable carrier; wherein each R₁ and R₂ may be independently selected from —F, —Cl, —Br, cyano, methyl, ethyl, and methoxy; and m and n may be independently an integer from 0 to 5. In one aspect, m and n may be 0.

In one aspect, the compound may comprise a compound having a structure selected from

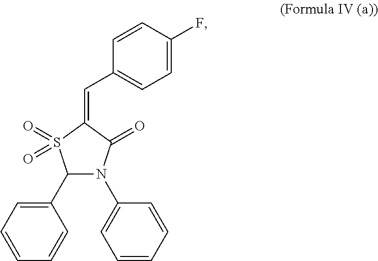 [SMILES: O=C1/C(=C\c2ccc(F)cc2)S(=O)(=O)C(c2ccccc2)N1c1ccccc1]

or a pharmaceutically acceptable salt thereof.

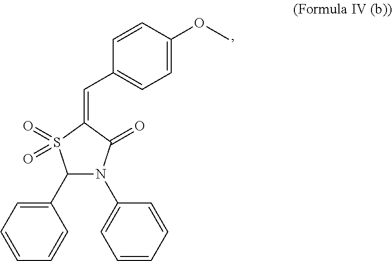 [SMILES: COc1ccc(/C=C2\C(=O)N(c3ccccc3)C(c3ccccc3)S2(=O)=O)cc1]

or a pharmaceutically acceptable salt thereof, and combinations thereof.

In one aspect, the compound may comprise Formula IV(c) as follows:

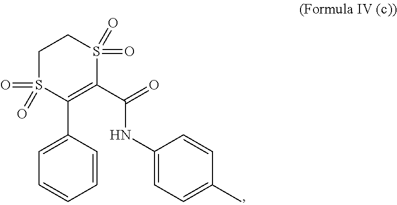 [SMILES: Cc1ccc(NC(=O)C2=C(c3ccccc3)S(=O)(=O)CCS2(=O)=O)cc1]

or a pharmaceutically acceptable salt thereof.

In one aspect, the compound may comprise Formula IV(d) as follows:

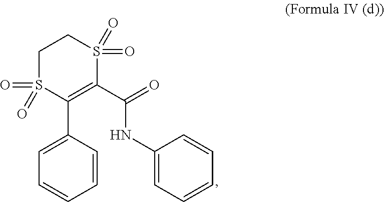 [SMILES: O=C(Nc1ccccc1)C1=C(c2ccccc2)S(=O)(=O)CCS1(=O)=O]

or a pharmaceutically acceptable salt thereof.

As such, in one aspect, the disclosed compounds may contain the related substructure as shown above.

In certain aspects, analogs or derivatives of the subject compounds are contemplated. For example, a chloromethyl group of a subject compound can be replaced with a methyl or difluoromethyl group. Exemplary analogs include tritiated analogs, biotinylated analogs, and analogs with photoactivatable cross-linkers. It is understood that methods of making structural analogs and derivatives of a compound are known and routine in the art.

### Identification of Derivative Compounds and Optimization Methods

The compounds listed herein and derivatives thereof may be useful for the treatment of NF1-related disorders or related pathways, such as, for example, malignant peripheral nerve sheath tumors.

Additional compounds may be identified via standard methods wherein the compounds herein, for example those of Table 1, are modified such that the overall activity at a biological target is expected to be preserved, while other properties are altered. Compounds predicted to have similar activity are first identified, then such compounds may be further selected or modified to achieve optimal or preferred “drug likeness,” as described herein. Additional compounds may be selected based on structural homology to the compounds described herein. Examples of such are provided in Table 6. Compounds predicted computationally to have related, similar, or analogous activity to the previous hits can be screened using the methods described herein. From the hits of both the initial and a secondary screen, directed screening of compounds closely related (“similar”) to these hits is carried out. A third screen, if desired, may be carried out.

Table 1 and the compounds listed in Table 6 and Table 7 may provide lead compounds which may provide a basis for identification of related compounds that may also be useful for the treatment the disclosed disorders. Identification of such compounds, and selection of preferred compounds, may be carried out in accordance with methods known to one of ordinary skill in the art. Such methods and principles are briefly described herein. This disclosure is not intended to be limiting, but rather, provide a general overview of methods by which additional compounds may be identified and selected as pharmaceutically acceptable compounds useful for the disclosed disorders. Modifications and alternatives to the methods described herein will be readily understood by one of ordinary skill in the art.

Three computational approaches may be used to identify similar compounds for screening in parallel. These are: 1) ROCS/EON Search: ROCS and EON are programs based on shape-matching and electrostatic matching algorithms that have shown solid track records of success in identifying highly similar and active compounds to a given active compound. Openeye Scientific Software, www.eyesopen.com, may be used to carry out this analysis; 2) Pipeline Pilot (Tanimoto) Similarity Search: Pipeline Pilot allows the simultaneous use of several Tanimoto Connectivity based similarity searches, which often identifies compounds of remarkable structural similarity; 3) Substructure Searches: From the literature available, one can identify key structural fragments that are important for activity and then identify compounds containing these structural features for screening.

In general, lead optimization, as known in the art, consists of the following general steps, which may be applied to the compounds and methods disclosed herein: From identification of a “hit,” a compound that is found to be active (i.e., exert a desired biochemical effect) in the initial screen, compounds are selected based on determination of IC50, EC50, or AC50 values. Hits are confirmed, as described above, either using the same or a different assay, particularly that of a functional assay or in a cellular environment. A second or third screen may be used to provide additional validation of function. Hits may then be evaluated according to their synthesis feasibility and other parameters such as up-scaling or costs. If the target is known, biophysical testing, such as nuclear magnetic resonance (NMR), isothermal titration calorimetry, dynamic light scattering, or surface plasmon resonance may be used to assess whether the compound binds effectively to the target, or to identify stoichiometry of the binding or the presence of promiscuous inhibitors. Confirmed hit compounds, such as those described in Table 3, can then be ranked according to the various hit confirmation experiments.

After confirmation of the initial hits, compounds may be clustered according to characteristics in the previously defined tests and/or overall similarity to the hit. In identifying a compound cluster, characteristics such as affinity towards the target (preferably less than 1 micromolar), chemical tractability, binding to the P450 enzymes, P-glycoproteins or serum albumin (wherein a lack of interference with these proteins are preferred), solubility in water, stability, membrane permeability, druglikeness, lack of cytotoxicity, metabolism (rapidly metabolized compounds are not preferred), and selectivity with an identified target. Compounds having preferred or optimal pharmacokinetic properties, ease of manufacture, solubility, safety, toxicity, metabolism, synthesis feasibility and other parameters such as up-scaling or costs, etc. may be determined

For example, from the list of compounds provided herein, one of ordinary skill in the art may apply standard methods and principles of medicinal chemistry to arrive at optimized compound structures that are preferred for administration to a mammal This can be done using a variety of different commercially available software packages or services which specialize in drug discovery, including lead discovery and optimization. See for example, Pharmacopeia Business Development, Princeton, N.J., which provides drug lead optimization services.

Structure-activity analysis may be conducted to identify core structures necessary for biological activity, such that additional compounds, derived from the initial hits shown in Table 3 or related compounds shown in Table 6, may be identified. Quantitative structure-activity relationship (QSAR) is the process by which chemical structure is quantitatively correlated with a well defined process, such as biological activity or chemical reactivity. For example, biological activity can be expressed quantitatively as in the concentration of a substance required to give a certain biological response. Additionally, when physiochemical properties or structures are expressed by numbers, one can form a mathematical relationship, or quantitative structure-activity relationship, between the two. The mathematical expression can then be used to predict the biological response of other chemical structures. The basic assumption for all molecule based hypotheses is that similar molecules have similar activities. This principle is also called Structure-Activity Relationship (SAR). It is well known for instance that within a particular family of chemical compounds, especially of organic chemistry, that there are strong correlations between structure and observed properties.

QSAR's most general mathematical form is:

Activity=f(physiochemical properties and/or structural properties)

3D-QSAR refers to the application of force field calculations requiring three-dimensional structures, e.g. based on protein crystallography or molecule superposition. It uses computed potentials, e.g. the Lennard-Jones potential, rather than experimental constants and is concerned with the overall molecule rather than a single substituent. It examines the steric fields (shape of the molecule) and the electrostatic fields based on the applied energy function. The created data space is then usually reduced by a following feature extraction (see also dimensionality reduction). The following learning method can be any of the already mentioned machine learning methods, e.g. support vector machines. The partial least squares (PLS) method may also be used, in which the feature extraction and induction is applied in one step.

3D-QSAR, referring to the application of force field calculations requiring three-dimensional structures, e.g. based on protein crystallography or molecule superposition, may also be used to predict preferred compounds. This method uses computed potentials, e.g. the Lennard-Jones potential, rather than experimental constants and evaluates the overall molecule rather than a single substituent. In this method, the steric fields (shape of the molecule) and the electrostatic fields based on the applied energy function are examined and optimized. See, for example, A. Leach, Molecular Modelling: Principles and Applications, Prentice Hall, 2001; SchOlkopf, B., K. Tsuda and J. P. Vert: Kernel Methods in Computational Biology, MIT Press, Cambridge, Mass., 2004; C. Helma (ed.), Predictive Toxicology, CRC, 2005; all incorporated herein in their entirety by reference. The created data space is then usually reduced by a following feature extraction (see also dimensionality reduction).

After compounds are selected based on the likelihood of the compound to exhibit similar bioactivity, such compounds may be further selected on the basis of druglikeness. While the compounds of the UC/GRI library are enriched for compounds having drug-like properties, the following analysis is applicable in identifying preferred compounds or in screening libraries which are not enriched for such compounds. “Druglikeness” refers to how druglike a substance is. This can be estimated from the molecular structure before the substance is synthesized and tested. A druglike molecule has properties such as optimal solubility to both water and fat, as an orally administered drug must pass through the intestinal lining, be carried in aqueous blood, and penetrate the lipid cellular membrane to reach the insider of a cell. The model compound for the cellular membrane is octanol, so the logarithm of the octanol/water partition coefficient, known as log Pow is used to estimate solubility. The compound can also be selected on the basis of overall water solubility, as therapeutic agents typically must be carried in aqueous media such as blood and intracellular fluid. Solubility in water can be estimated from the number of hydrogen bond donors versus alkyl sidechains in the molecule. Low water solubility translates to slow absorption and action. Too many hydrogen bond donors, on the other hand, lead to low fat solubility, so that the drug cannot penetrate the cell wall reach the inside of the cell. Druglike substances are also those that are relatively small in molecular weight, as this parameter determines diffusion. Compounds less than about 1000 Daltons, or about 800 daltons, or about 500 daltons, or about 450 daltons may be used. 80% of traded drugs have molecular weights under 450 daltons. Druglikeness is also determined based on the presence of substructures that have known pharmacological properties.

As a means of predicting general druglikeness, “Lipinski's Rule of Five” may be used. This rule allows one to generally determine if a chemical compound with pharmacological or biological activity has properties that would make it a likely orally active drug in humans. This rule is based on the general observation that most therapeutic agents are relatively small and lipophilic molecules. The rule describes molecular properties important for a drug's pharmacokinetics in the human body, including absorption, distribution, metabolism and excretion (“ADME”). In addition to evaluating identified compound clusters, this rule may be used to modify or optimize a lead structure step-wise for increased drug-like properties. For example, these principles may be applied to modify the molecular structure of a compound in the compound cluster or modification of a hit or lead compound to arrive at compounds having ideal molecular weights, rings, bonds, or lipophilicity. Lipinski's Rule of Five (all numbers in the rule are multipliers of the number 5) states that, in general, an orally active drug has: 1) not more than 5 hydrogen bond donors (OH and NH groups), 2) not more than 10 hydrogen bond acceptors (notably N and O); 3) a molecular weight under 500 g/mol; 4) a partition coefficient log P less than 5. See C. A. Lipinski, F. Lombardo, B. W. Dominy, P. J. Feeney, Experimental and computational approaches to estimate solubility and permeability in drug discovery and development settings, Adv. Drug Del. Rev., 2001, 46, 3-26, incorporated herein by reference. Software for calculating properties and predicting bioactivity of a compound is readily available, for example, at www.molinspiration.com. The compounds may be further optimized according to guidelines set forth in Ghose, et al. (1999). These are: partition coefficient log P in −0.4 to +5.6 range; molar refractivity from 40 to 130; molecular weight from 160 to 480; number of heavy atoms from 20 to 70. In addition to application of the Rule of Five, preferred compounds may be selected based on the predicted ADME. ADME refers to absorption, distribution, metabolism or excretion; compounds may be selected as preferred compounds for additional screening or testing for efficacy as a therapeutic compound on this basis. QSPR or QSAR may be used to predict the ADME and toxicity of a compound.

Based on an assessment of this information, druglikeness indexes can be constructed based on molecular fragments of structures (Xu and tevenson 2000). The “drug like index” (DLI) is may be constructed according to a formula that uses the true and false positives, or true and false negatives in any set of best results that were obtained using the types of data described above. The DLI may be used for prioritizing molecules in any set of given structures, such as within the data sets of molecules obtained via High Throughput Screening (HTS) for molecular hits, in preparing lists of combinatorial chemistry for synthesis, or in assigning structures for High Throughput in Silico Docking of molecules, or those compound clusters described herein. The DLI may be further used for optimization of identified compounds (such as those listed herein) toward viable pharmaceutical agents by combinatorial addition of substituents that optimize their drug likeness. Using computational docking experiments as known in the art, DLI may also be combined with scores for the affinity. DLI may be used to decide how to reduce compound sets so that smaller sets could be examined (by HTS) or synthesized (by Combinatorial Chemistry). In summary, the DLI allows stratification of compounds such one may readily select compounds likely to be useful as therapeutic agents in practice. After selection of these compounds, routine testing of these compounds, including in vitro and in vivo testing, may be carried out.

In accordance with the above-described methods, US 2007/0156343, Rayan et al., filed Oct. 24, 2004, is incorporated in its entirety by reference.

### Compositions

In another aspect, compositions that comprise a safe and effective amount of a subject compound, or a pharmaceutically-acceptable salt thereof, and a pharmaceutically-acceptable carrier are disclosed. In addition to the subject compound, the compositions may comprise a pharmaceutically-acceptable carrier. Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitcl, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogenfree water; isotonic saline; and phosphate buffer solutions. The choice of a pharmaceutically-acceptable carrier to be used in conjunction with the subject compound is basically determined by the way the compound is to be administered. If the subject compound is to be injected, the preferred pharmaceutically-acceptable carrier is sterile, physiological saline, with a blood-compatible suspending agent, the pH of which has been adjusted to about 7.4.

If the mode of administering the subject compound is perorally, the preferred unit dosage form is therefore tablets, capsules, lozenges, chewable tablets, and the like. Such unit dosage forms comprise a safe and effective amount of the subject compound, which is preferably from about 0.01 mg to about 350 mg, more preferably from about 0.1 mg to about 35 mg, based on a 70 kg person. The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of this disclosure, and can be readily made by a person skilled in the art.

Peroral compositions also include liquid solutions, emulsions, suspensions, and the like. The pharmaceutically-acceptable carriers suitable for preparation of such compositions are well known in the art. Such liquid oral compositions preferably comprise from about 0.001% to about 5% of the subject compound, more preferably from about 0.01% to about 0.5%. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel®RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the subject compounds include sublingual and buccal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included. Compositions can also be used to deliver the compound to the site where activity is desired: intranasal doses for nasal decongestion, inhalants for asthma, and eye drops, gels and creams for ocular disorders. Compositions may include solutions or emulsions, preferably aqueous solutions or emulsions comprising a safe and effective amount of a subject compound intended for topical intranasal administration. Such compositions preferably comprise from about 0.001% to about 25% of a subject compound, more preferably from about 0.01% to about 10%. Similar compositions are preferred for systemic delivery of subject compounds by the intranasal route. Compositions intended to deliver the compound systemically by intranasal dosing preferably comprise similar amounts of a subject compound as are determined to be safe and effective by peroral or parenteral administration. Such compositions used for intranasal dosing also typically include safe and effective amounts of preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfate and others; aromatic agents; viscosity adjustors, such as polymers, including cellulose and derivatives thereof, and polyvinyl alcohol and acids and bases to adjust the pH of these aqueous compositions as needed. The compositions may also comprise local anesthetics or other actives. These compositions can be used as sprays, mists, drops, and the like.

Other compositions may include aqueous solutions, suspensions, and dry powders comprising a safe and effective amount of a subject compound intended for atomization and inhalation administration. Such compositions may comprise from about 0.1% to about 50% of a subject compound, more preferably from about 1% to about 20%; of course, the amount can be altered to fit the circumstance of the patient contemplated and the package. Such compositions are typically contained in a container with attached atomizing means. Such compositions also typically include propellants such as chlorofluorocarbons 12/11 and 12/114, and more environmentally friendly fluorocarbons, or other nontoxic volatiles; solvents such as water, glycerol and ethanol, these include cosolvents as needed to solvate or suspend the active; stabilizers such as ascorbic acid, sodium metabisulfite; preservatives such as cetylpyridinium chloride and benzalkonium chloride: tonicity adjustors such as sodium chloride; buffers; and flavoring agents such as sodium saccharin. Such compositions are useful for treating respiratory disorders, such as asthma and the like.

Other compositions may include aqueous solutions comprising a safe and effective amount of a subject compound intended for topical intraocular administration. Such compositions preferably comprise from about 0.0001% to about 5% of a subject compound, more preferably from about 0.01% to about 0.5%. Such compositions also typically include one or more of preservatives, such as benzalkonium chloride, thimerosal, phenylmercuric acetate; vehicles, such as poloxamers, modified celluloses, povidone and purified water; tonicity adjustors, such as sodium chloride, mannitol and glycerin; buffers such as acetate, citrate, phosphate and borate; antioxidants such as sodium metabisulfite, butylated hydroxy toluene and acetyl cysteine; acids and bases may be used to adjust the pH of these formulations as needed.

Other compositions useful for peroral administration may include solids, such as tablets and capsules, and liquids, such as solutions, suspensions and emulsions (preferably in soft gelatin capsules), comprising a safe and effective amount of a subject compound. Such compositions preferably comprise from about 0.01 mg to about 350 mg per dose, more preferably from about 0.1 mg to about 35 mg per dose. Such compositions can be coated by conventional methods, typically with pH or time-dependent coatings, such that the subject compound is released in the gastrointestinal tract at various times to extend the desired action. Such dosage forms typically include one or more of cellulose acetate phthalate, polyvinylacetate phthalate, hydroxypropyl methyl cellulose phthalate, ethyl cellulose, Eudragit® coatings, waxes and shellac. Any of the disclosed compositions may optionally include other drug actives.

While specific aspects have been discussed, the above specification is illustrative and not restrictive. Many variations will become apparent to those skilled in the art upon review.

### Test Methods/Assays

The following assays and protocols are employed in carrying out the above-described methods. Variations to these methods will be understood by one of ordinary skill in the art, and such variations are not intended to be excluded from the scope of the disclosure.

MPNST tumor acquisition and processing—Tumor specimens and corresponding clinical data were collected and used in accordance with Institutional Review Board-approved protocols. The diagnosis of NF1 was established according to published criteria (NIH Consensus Statement). Frozen, archived tumor specimen pathology was reviewed and RNA isolated and then analyzed on Affymetrix U95Av2 GeneChip microarrays as reported (18).

Cell culture —Human MPNST cell lines are collected from patients with and without NF1 mutations. T265p21, 90-8, ST88-14, 88-3, and STS26T cell lines were provided by Jeff DeClue (National Cancer Institute, Bethesda, Md.). The YST-1 cell line was provided by Yoji Nagashima (University School of Medicine, Yokohama, Japan). Human Narf cells that express isopropyl-L-thio-B-Dgalactopyranoside (IPTG)-inducible human ADP ribosylation factor (ARF) were provided by Gordon Peters (Imperial Cancer Research Fund, London, United Kingdom). ST88-14, STS26T, 5520, 5462, and Narf lines were grown in DMEM (Fisher Scientific, Pittsburgh, Pa.) supplemented with 10% fetal bovine serum (FBS; Harlan, Indianapolis, Ind.). 88-3, 90-8, and T265p21 lines were grown in a RPMI 1640-based medium as described (7). The YST-1 line was grown in RPMI 1640 (Fisher Scientific) containing 10% FBS. NHSCs were generated as previously described (19). Analyses were done under standard culture conditions for each cell line. Several assays were not conducted on the 88-3 cells due to difficulties in culturing this line.

NF1 mutation analysis—DNA was isolated from frozen STS26T or YST1 cell pellets. The NF1 gene was screened for mutations by denaturing high-performance liquid chromatography-based heteroduplex analysis using the WAVE analysis system (Transgenomic, Omaha, Nebr.) as described. Several primer sequences were redesigned to reduce their homology to the NF1 pseudogenes sequences (20).

3-(4,5-Dimethylthiazol-2-y0-2,5-diphenyltetrazolium bromide assay —Cells (5×103) were plated in triplicate on a 24-well plate (day 0). The 3-(4,5-dimethylthiazol-2-yl)-2,5-diphenyltetrazolium bromide (MTT) assay was done on day 1 and day 4 by adding 50 pL of a 5 mg/mL MTT solution (Sigma-Aldrich, St. Louis, Mo.) to each well. Following incubation at 37° C. for 2 hours, the formazan precipitate was extracted in isopropanol-HCl and absorbance measured at 540 nm. For Taxol sensitivity assays, the noted concentration of paclitaxel (Sigma-Aldrich) was added to the medium on day 1. Concentration of paclitaxel was based on previous studies (21).

Western blot analysis —Cells were lysed on ice in 50 mmol/L Tris (pH 7.5), 120 mmol/L NaCl, 1 mmol/L EDTA, 0.5% NP40, 0.1 mmol/L sodium vanadate, 1 mmol/L sodium flouride, 5 ug/mL leupeptin, and 30 umol/L phenylmethylsulfonyl flouride. Lysates were sonicated and clarified by centrifugation. Equivalent amounts of protein (50-100 pig) were separated by electrophoresis on SDSpolyacrylamide gradient gels (7-15% or 4-20%; ISC BioExpress, Kaysville, Utah) and transferred to polyvinylidene difluoride membrane (Bio-Rad, Hercules, Calif.). Membranes were probed with anti-ARF (NB 200-111; Novus Biologicals, Littleton, Colo.), anti-RB (clone G3-245; BD Biosciences PharMingen, San Diego, Calif.), anti-p16 (Ab-1; Research Products, San Diego, Calif.), anti-p53 (DO-1; Santa Cruz Biotechnology, Santa Cruz, Calif.), anti-HDM2 (2A10 hybridoma supernatant provided by Gerry Zambetti, St. Jude Children's Research Hospital, Memphis, Term., and Arnold Levine, Cancer Institute of New Jersey/UMDNJ, New Brunswick, N.J.), or anti-TWIST1 antibodies (22). Blots were stripped and reprobed with antitubulin (Sigma-Aldrich) or anti-β-actin (Cell Signaling Technology, Inc., Danvers, Mass.) as a loading control. Signals were detected using horseradish peroxidase-conjugated secondary antibodies (Bio-Rad, Hercules, Calif.) in combination with Enhanced Chemiluminescence (ECL) Plus developing system (Amersham Biosciences) according to the specifications of the manufacturer. For TWIST1 quantification, ImageJ 1.33u software (http://rsb.info.nih.gov/ij/) was used to obtain values from scanned autoradiographs representing protein levels. TWIST1 levels were normalized to 13-actin levels for each sample.

Immunohistochemistry—Cell suspensions were mounted in a HistoGel (Richard Allan-Scientific, Kalamazoo, Mich.) “button” according to the protocol of the manufacturer and embedded in paraffin blocks. S100B and EGFR protein expression was determined by a semiautomated immunohistochemical technique as described (Ventana ES, Ventana Medical Systems, Tuscon, Ariz.; ref 14).

Mouse Schwann cell culture —NF1−/− embryos obtained from timed mating of NF1+/− C57B1/6 male and NF1+/− 129 female mice at embryonic day 12.5 are identified by PCR genotyping (Brannan et al., 1994). Mouse Schwann cells (MSCs) are isolated from embryonic day 12.5 dorsal root ganglia (Kim et al., 1995). Schwann cells are cultured on poly-L-lysine-coated plates in DMEM with 10% fetal bovine serum, 10 ng/ml 131 heregulin peptide (R&D Systems) and 21.tM forskolin (Calbiochem). Cells are used between passages 1 and 3. For conditioned medium, Schwann cell cultures at 90% confluence are switched to N2 medium (DMEM/F12+N2 supplements, GibcoBRL) and incubated for 48 hours. Conditioned medium is harvested, centrifuged, and stored at −70° C. Viability Assay. Cells are cultured on poly-L-lysine in 96 well plates, 48-72 hours after plating 10,000 cells per well. Cell numbers are determined indirectly using an MTS assay. All assays are performed in triplicate. If differences in viability are observed, follow up assays will measure BrdU incorporation and cell death.

Migration assay—The migratory response of MPNST cells are measured using a modified Boyden chamber assay. 4×10⁴ cells in serum-free DMEM are plated on the upper chamber of a transwell with 8-um pores (Costar, Corning Inc., Corning, N.Y.). The lower chamber contains 800 uL MPNST conditioned medium. Cells are incubated for 16 hours at 37° C. in 10% CO2. Nonmigrating cells were removed from the upper surface of the membrane with cotton swabs. Membranes were stained with bisbenzimide and mounted onto glass slides. Migration was quantified by counting cells in four fields. Each condition was done in triplicate and the number of migrated cells was normalized to the total number of cells on an unscraped filter to validate the total number of cells plated. Data shown are representative of three independent experiments; values presented are the mean±SD. Statistical significance was determined by t test using Microsoft Excel software.

Bromodeoxyuridine incorporation —Twenty-four hours post plating 3×10⁴ cells onto glass coverslips, cells were labeled for 1 hour with bromodeoxyuridine (BrdUrd) labeling reagent (1:1,000; Amersham Biosciences, Piscataway, N.J.) to detect DNA synthesis. Cells were fixed with 3.7% formaldehyde (Fisher Scientific), permeabilized in 0.3% Triton X-100 (Sigma-Aldrich), and incubated with anti-BrdUrd antibodies (1:200; Accurate Chemical & Scientific, Westbury, N.Y.) in immunofluorescence buffer [20 mmol/L MgCl2, 50 units DNase I (Roche, Basel, Switzerland)] for 45 minutes at 37° C. Cell nuclei and BrdUrd localization were visualized by incubating in 5 lig/mL bisbenzamide (Sigma-Aldrich) and rhodamine-conjugated donkey anti-Rat secondary antibodies (1:100; Jackson ImmunoResearch, West Grove, Pa.). Total number of cells and number of BrdUrd-positive cells were counted in five fields per sample and averaged. Assays were done in duplicate.

Viability assay —Cells were cultured on glass coverslips. Live versus dead cell numbers were determined using a LIVE/DEAD Viability/Cytotoxicity kit (Molecular Probes, Eugene, Oreg.) according to the protocol of the manufacturer. The total number of live and dead cells was counted in five fields per sample and averaged. Assays were done in duplicate.

Determination of LD50—Mice are injected interperitoneally at an initial dose which is that of the IC50, increasing in ⅓ Log steps, to determine the lethal dose (LD50). The dose at which toxic effects are noted can also be determined to ensure that the “therapeutic” dose is well below the toxic dose. Necropsy can be performed on animals that received confirmed hit compounds to evaluate for possible toxic effects on animal organs. Tissues obtained will include brain, spinal cord, heart, lungs, spleen, liver, large intestines, muscle, bone, and bone marrow. (2) A mass spectroscopy test for compounds in plasma will can also be developed.

### EXAMPLES

The following non-limiting example illustrates an embodiment of the methods disclosed herein.

### Example I

### Initial Screen

Candidate compounds are tested on the T265p21 (NFI) and STS26T (non-NF1) cell lines. Although many MPNST cell lines differ in growth rates, these two lines show similar, vigorous growth in vitro (FIG. 1B). MPNST cell lines are shown in Table 2. Patient information and histopathology of the primary tumors is documented in Nagashima et al. 1990; Fletcher et al. 1991; Dahlberg et al. 1993; Frahm et al. 2004. The sporadic MPNST lines, STS26T and YST-1, are wild type at the NF1 locus as identified by screening all 50 exons of the NF1 gene for mutations using denaturing high-performance liquid chromatography based heteroduplex analysis. Consistent with the role of NF1 as a RasGAP, only the NF1 patient derived, NFI mutant cell lines show increased Ras-GTP which can be blocked by the gap-related domain of NF1 (FIG. 1A). Similarly, the MAPK cascade downstream of Ras-GTP is activated in NF1 mutant cells and blocked by the GRD.

TABLE 2
MPNST Cell Lines - “+” indicates documented history
of NF1 disease. “*” indicates no documented history
of NF1 disease; “*” indicates a cell line with a
microdeletion of NF1 (Wu et al. 1999). LOH = loss of
heterozygosity region of the NF1 gene, previously confirmed
in five of the six NF/-associated MPNST lines (Glover et
al. 1991; Reynolds et al. 1992; Wu et al. 1999).

| Cell Line | NF1 Patient | Reference |
|---|---|---|
| ST88-14 | +(LOH) | Fletcher et al., 1991 |
| 90-8 | +(LOH) * | Glover et al., 1991 |
| 88-3 | +(LOH) | Glover et al., 1991 |
| T265p21 | + | Badache and DeVries, 1998 |
| S462 | +(LOH) | Frahm et al., 2004 |
| S520 | +(LOH) | Frahm et al., 2004 |
| STS26T | | Dahlberg et al., 1993 |
| YST-1 | − | Nagashima et al., 1990 |

The NF1−/− MPNST line T265 used for the screen provides two additional readouts for the chemical approaches to suppress the phenotypes of NFI loss: activated Ras (Mahller et al., 2005) and activated MAPK (FIG. 1). In vitro growth assays of the two cell lines (NF1 positive and NF1 negative) are scaled down to 384 well plates. An Evotec Technologies plate::explorer uHTS system is used. MPNST cell lines T265 and STS26T in logarithmic growth phase are plated in separate 384 well plates at a density of 500 cells per well in Dulbecco's modified Eagle's medium (DMEM; Fisher Scientific; Pittsburgh, Pa.) supplemented with 10% Fetal Bovine Serum (Harlan; Indianapolis, Ind.) containing vehicle (DMSP, “Dimethyl Sulfoxide”) only or one of 13,031 candidate compounds at a concentration of 10 micromolar, a concentration of drug shown not to cause cell death of many human cell lines. The candidate compounds are selected to represent a cross section of compounds covering the diversity space resident in the available library, restricted to drug-like compounds, but evenly and consistently representative across this realm and representative of commercially available compounds. Compounds targeting GPCRs, kinases and other targets of known medical interest are also included. The cells are incubated with the compounds or 5 μg/ml doxorubicin as a positive control for 2 days. Growth is monitored by using the alamar blue method (Alley et al. 1988; Ahmed et al. 1994.) Metabolic activity of cells results in reduction of alamar blue and a large increase in alamar blue fluorescence, which correlates with cell number. Candidate compounds causing interference with fluorescence are eliminated. Candidate compounds causing fluorescence interference are eliminated. Appropriate control samples and statistical analyses are used.

Candidate compounds that cause differences in growth in the two cell lines, i.e., compounds that slowed down or stopped growth of the NF1−/− but not the NF1+1+ MPNST cells are considered “hits” and potential therapeutic agents for the treatment of NF/-associated disorders. The migration assay, viability assay, bromodeoxyuridine assay, described in detail below, or any other assay known in the art to measure cell growth, number, viability, or migration may be used to determine the effect on the NF1−/− and NFI+/+MPNST cell lines.

Confirmed hits that decreased viability of both cell lines were identified as possible therapeutic agents. A smaller population of hits selectively affected the non-NF1 cell line. Seven candidate agents showed selectivity for the NF1 cell line and are shown in Table 3. These seven confirmed “hits,” or potential therapeutic agents, were tested using the same assay method at 10 concentrations, in triplicate, to give a dose-response and to determine the AC50 (the concentration at which 50% of the maximum activity or inhibition is achieved) for each compound. Using the initial screen, seven compounds are identified showing a significant differential growth inhibition of the mutant NF1 cells relative to wild type NF1 cells.

TABLE 3
Confirmed hit compounds showing selectivity for the NF1 cell
line.

| | | 26T | |
|---|---|---|---|
| Formula | | IC50 | T265 |
| Number | Compound Structure | (μM) | IC50 |
| Ia | 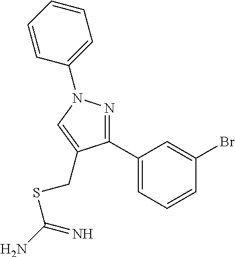 [SMILES: N=C(N)SCc1cn(-c2ccccc2)nc1-c1cccc(Br)c1] | 50.3 | 1.3 |
| III | 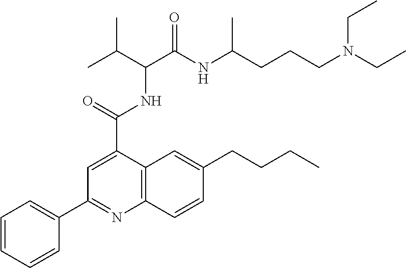 [SMILES: CCCCc1ccc2nc(-c3ccccc3)cc(C(=O)NC(C(=O)NC(C)CCCN(CC)CC)C(C)C)c2c1] | No Effect | 3.0 |
| V | 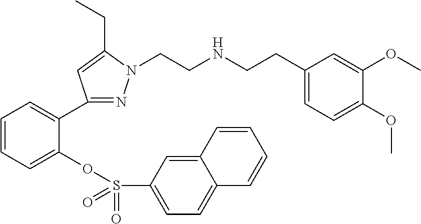 [SMILES: CCc1cc(-c2ccccc2OS(=O)(=O)c2ccc3ccccc3c2)nn1CCNCCc1ccc(OC)c(OC)c1] | 22.8 | 2.8 |
| VI | 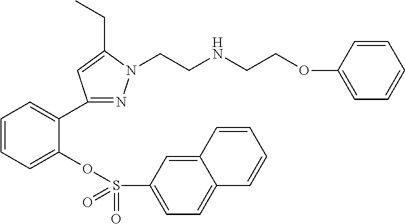 [SMILES: CCc1cc(-c2ccccc2OS(=O)(=O)c2ccc3ccccc3c2)nn1CCNCCOc1ccccc1] | 1.9 | 4.5 |
| VII | 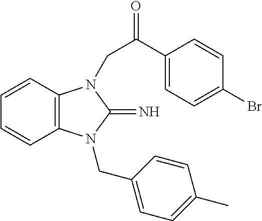 [SMILES: Cc1ccc(Cn2c(=N)n(CC(=O)c3ccc(Br)cc3)c3ccccc32)cc1] | 0.056 | 0.078 |
| VIII | 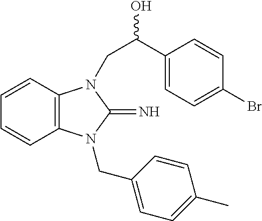 [SMILES: Cc1ccc(Cn2c(=N)n(CC(O)c3ccc(Br)cc3)c3ccccc32)cc1] | 19.5 | 1.1 |
| XI | 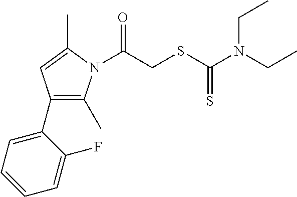 [SMILES: CCN(CC)C(=S)SCC(=O)n1c(C)cc(-c2ccccc2F)c1C] | 1.0 | 1.7 |

Of the seven, four showed robust differential effects in the NFI mutant sarcoma cells (T265) and the NF1 wild type (26T) sarcoma cells. These differences are shown in Table 4. Candidate agents meeting criteria for a hit, or potential therapeutic agent, are tested three additional times at the same concentration as the initial assay. Candidate agents that met assay criteria for a hit in at least two of the three replicates were designated “confirmed” HTS hits.

TABLE 4
IC50 values of hit compounds showing differential
growth inhibition in NF1 mutant sarcoma cells (T265)
and the NF1 wild type (26T) sarcoma cells.

| | | |
|---|---|---|
| Compound | IC50, μM | Fold |

| Formula No. | 26T | T265 | Difference |
|---|---|---|---|
| Ia | 50.3 | 1.3 | 38.7 |
| V | 22.9 | 2.9 | 8.1 |
| X | No Effect | 3.0 | ∞ |
| VIII | 19.5 | 1.1 | 17.7 |

A search of the chemical literature produced no references specifically describing the structure of Formula Ia, although the structure is known as a commercially available entity. The compound lacking the bromine group is the closest published relative. This and other compounds related to Formula Ia have been described as intermediates in synthesis (Russ. J. Org. Chem. 2002). Similar structures can be seen as a substructure within other more complex compounds (e.g. Chem. Pharm. Bull. 2003), the closest of which is shown below and described as having antiangiogenic activity.

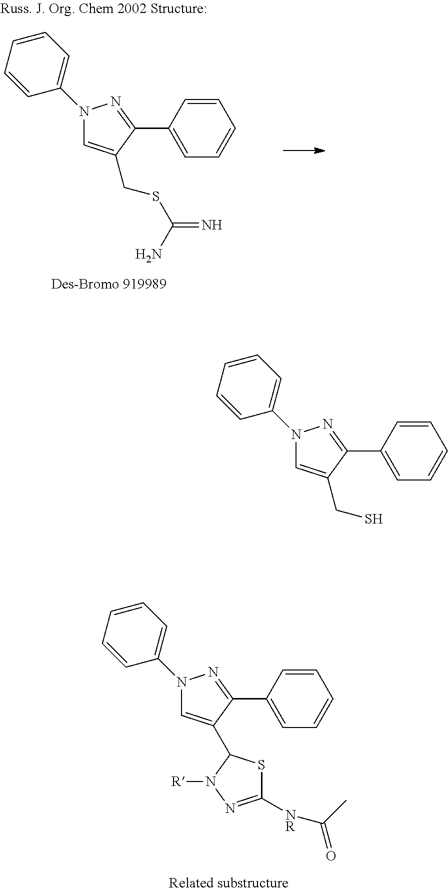

### Example II

### Validation of Hit Compounds Using MPNST Cells

Compounds including confirmed hits and additional confirmed hits identified using the protocols described in Example I are tested on three additional NF1MPNST cell lines and one non-NF/MPNST cell line (See Table 2 and FIG. 1). Assays, such as the viability and migration assay, are carried out for two days (as in the high throughput screen), and for one week, to determine if longer exposures show improved efficacy. The compounds that block growth selectively in NF1+/+ MPNST cells are then further validated in lab-based studies using primary mouse Nf1+/+ and NF1−/− Schwann cells established from mice. These cells differ only in NF1 status, while MPNST cells have sustained multiple genetic alterations. Viability and cell migration assays as described above are used, as these assays are able to detect differences between NFI mutant and wild type Schwann cells Inhibitors of known signaling pathways have only partial effects on NF1 Schwann cell migration (Huang et al., 2003). These assays are described above and reviewed in Ratner et al., 2006. Each compound is tested at four doses, above and below the AC50 defined for MPNST cells. Assays are carried out in triplicate.

### Example III

### Identification of Additional Compounds

A similarity search based on four distinct connectivity indices was performed, with each method selecting the 400 “most” similar compounds from the library. The combined methods identified 982 similar compounds. 428 compounds were identified by two or more protocols; 138 compounds were identified in 3 or more of the protocols and 56 compounds were identified by all four protocols. The resulting compound set is included as Table 6. Of the 982 similar compounds identified above, 44 of these were part of the original screening set (none identified as active). Similarity assessments may be a highly “approximate” determination; a combination of parameters (connectivity indices) to allow the methods to compensate for each one's strengths and weaknesses is used. A substructure search of the diphenylpyrazole core was conducted. 390 compounds containing this substructure exist in the library used, and are set forth in Table 7. Twelve of these compounds share the diphenyl pyrazole core structure but were demonstrated to be inactive using the above-described NF1 screen (see Table 5). One of skill in the art will recognize that knowledge of inactive compound provides data as to which functional groups of a molecule in this set are unlikely to contribute to the therapeutic effect observed in the compounds of Table 3, and such data can be used, for example, using computational methods, to identify and stratify compounds such that a pool of molecules may be enriched for those more likely to exert a biologically desirable effect.

As such, these compounds, and other compounds having the diphenyl pyrazole core structure are also considered viable candidates for the disclosed disorders.

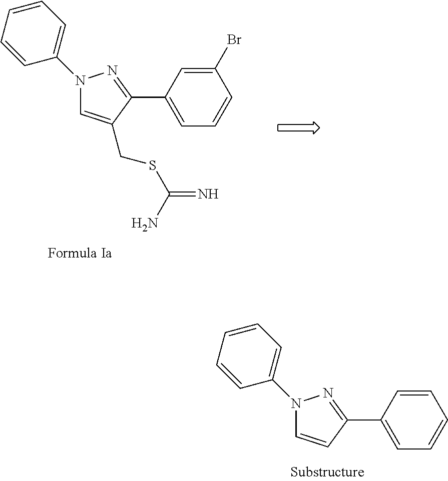 [SMILES: N=C(N)SCc1cn(-c2ccccc2)nc1-c1cccc(Br)c1 ; c1ccc(-c2ccn(-c3ccccc3)n2)cc1]

Table 6 lists example compounds identified using a similarity analysis using Formula Ia as the base compound. The compounds of Table 6 were identified based on a similarity search using four distinct connectivity indices, with each method selecting the 400 “most” similar compounds from the Compound Library available at UC/GRI. The combined methods identified 982 similar compounds. 428 compounds were identified by two or more protocols; 138 compounds were identified in 3 or more of the protocols and 56 compounds were identified by all four protocols. The 138 compound set is included in Table 6.

TABLE 5
Inactive Compounds having the diphenyl pyrazole core substructure

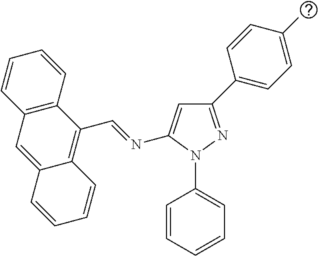 [SMILES: Brc1ccc(-c2cc(/N=C/c3c4ccccc4cc4ccccc34)n(-c3ccccc3)n2)cc1]

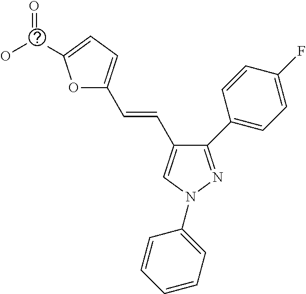

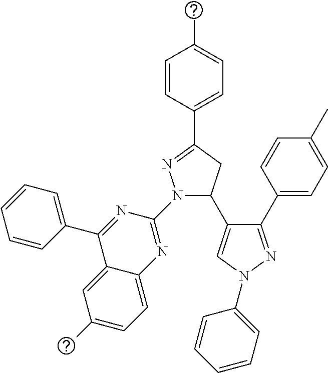 [SMILES: Cc1ccc(-c2nn(-c3ccccc3)cc2C2CC(c3ccc(Br)cc3)=NN2c2nc(-c3ccccc3)c3cc(O)ccc3n2)cc1]

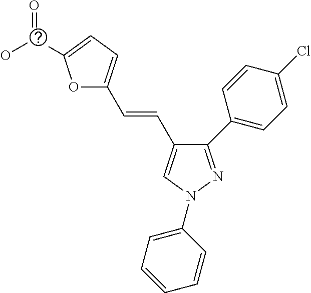

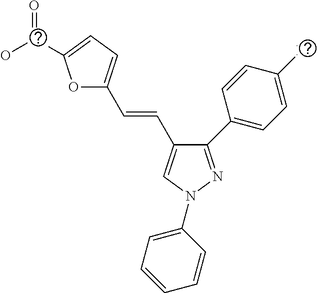

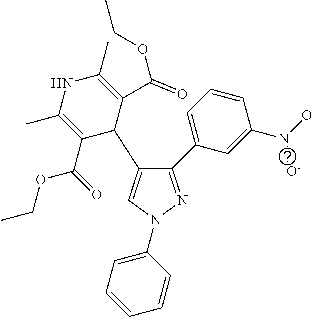

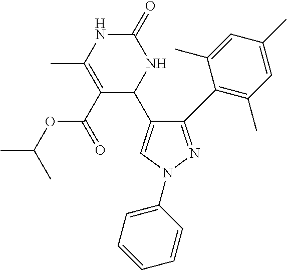 [SMILES: CC1=C(C(=O)OC(C)C)C(c2cn(-c3ccccc3)nc2-c2c(C)cc(C)cc2C)NC(=O)N1]

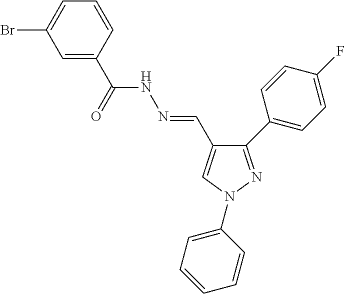 [SMILES: O=C(N/N=C/c1cn(-c2ccccc2)nc1-c1ccc(F)cc1)c1cccc(Br)c1]

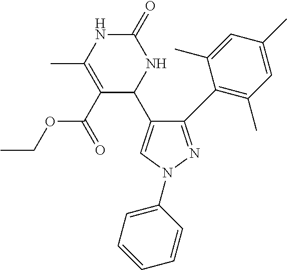 [SMILES: CCOC(=O)C1=C(C)NC(=O)NC1c1cn(-c2ccccc2)nc1-c1c(C)cc(C)cc1C]

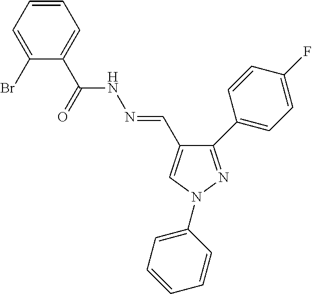 [SMILES: O=C(N/N=C/c1cn(-c2ccccc2)nc1-c1ccc(F)cc1)c1ccccc1Br]

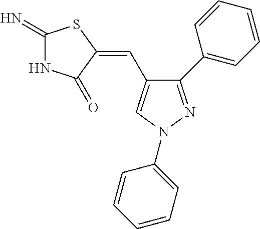 [SMILES: N=C1NC(=O)/C(=C\c2cn(-c3ccccc3)nc2-c2ccccc2)S1]

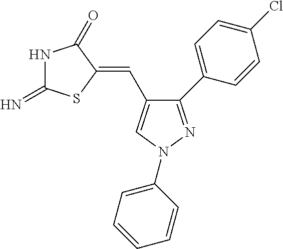 [SMILES: N=C1NC(=O)/C(=C/c2cn(-c3ccccc3)nc2-c2ccc(Cl)cc2)S1]

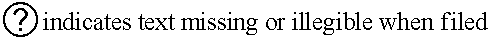

TABLE 6
Example Compounds from Similarity Analysis

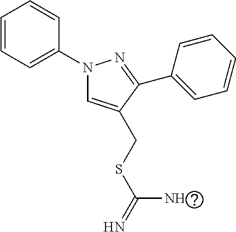 [SMILES: N=C(N)SCc1cn(-c2ccccc2)nc1-c1ccccc1]

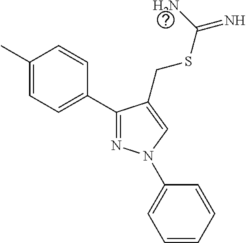 [SMILES: Cc1ccc(-c2nn(-c3ccccc3)cc2CSC(=N)N)cc1]

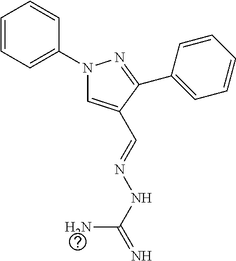 [SMILES: N=C(N)N/N=C/c1cn(-c2ccccc2)nc1-c1ccccc1]

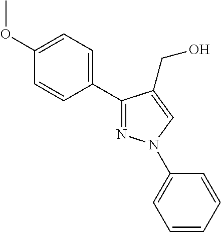 [SMILES: COc1ccc(-c2nn(-c3ccccc3)cc2CO)cc1]

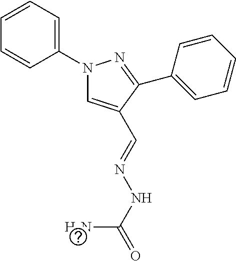 [SMILES: NC(=O)N/N=C/c1cn(-c2ccccc2)nc1-c1ccccc1]

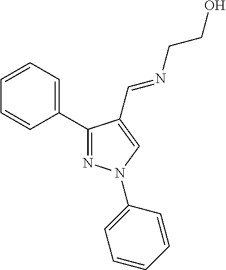 [SMILES: OCC/N=C/c1cn(-c2ccccc2)nc1-c1ccccc1]

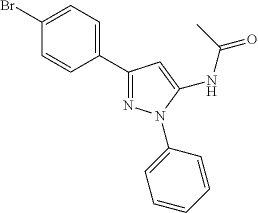 [SMILES: CC(=O)Nc1cc(-c2ccc(Br)cc2)nn1-c1ccccc1]

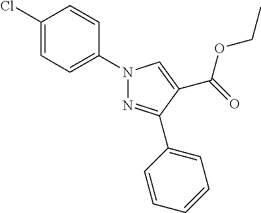 [SMILES: CCOC(=O)c1cn(-c2ccc(Cl)cc2)nc1-c1ccccc1]

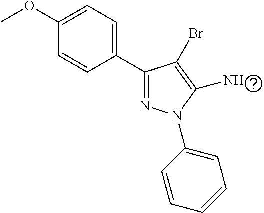 [SMILES: COc1ccc(-c2nn(-c3ccccc3)c(N)c2Br)cc1]

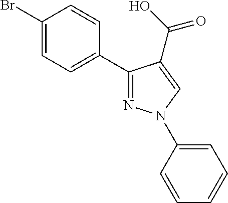 [SMILES: O=C(O)c1cn(-c2ccccc2)nc1-c1ccc(Br)cc1]

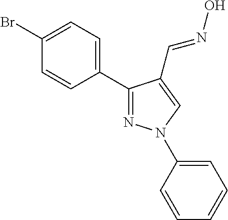 [SMILES: O/N=C/c1cn(-c2ccccc2)nc1-c1ccc(Br)cc1]

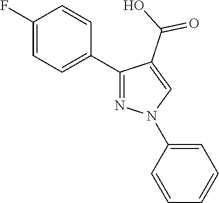 [SMILES: O=C(O)c1cn(-c2ccccc2)nc1-c1ccc(F)cc1]

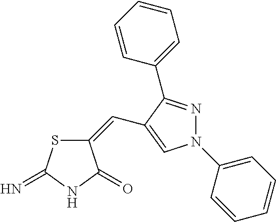 [SMILES: N=C1NC(=O)/C(=C\c2cn(-c3ccccc3)nc2-c2ccccc2)S1]

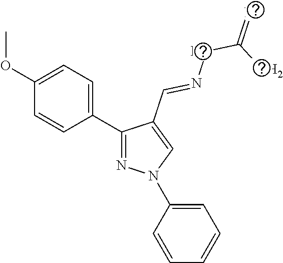 [SMILES: COc1ccc(-c2nn(-c3ccccc3)cc2/C=N/NC(=N)N)cc1]

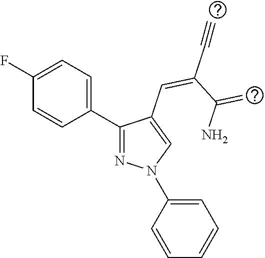 [SMILES: N#C/C(=C/c1cn(-c2ccccc2)nc1-c1ccc(F)cc1)C(N)=S]

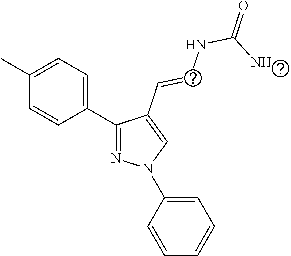 [SMILES: Cc1ccc(-c2nn(-c3ccccc3)cc2/C=N/NC(N)=O)cc1]

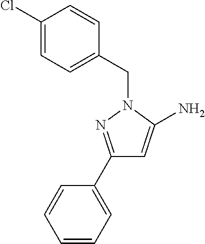 [SMILES: Nc1cc(-c2ccccc2)nn1Cc1ccc(Cl)cc1]

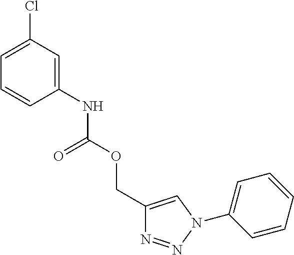 [SMILES: O=C(Nc1cccc(Cl)c1)OCc1cn(-c2ccccc2)nn1]

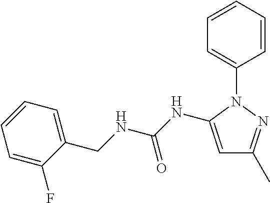 [SMILES: Cc1cc(NC(=O)NCc2ccccc2F)n(-c2ccccc2)n1]

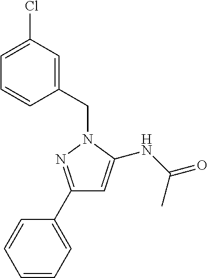 [SMILES: CC(=O)Nc1cc(-c2ccccc2)nn1Cc1cccc(Cl)c1]

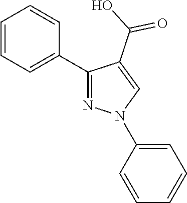 [SMILES: O=C(O)c1cn(-c2ccccc2)nc1-c1ccccc1]

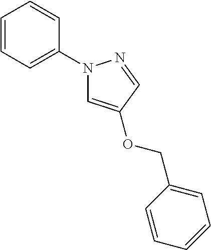 [SMILES: c1ccc(COc2cnn(-c3ccccc3)c2)cc1]

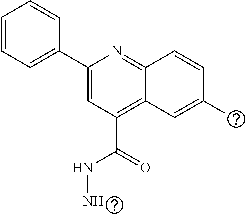 [SMILES: NNC(=O)c1cc(-c2ccccc2)nc2ccc(Br)cc12]

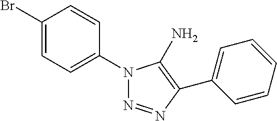 [SMILES: Nc1c(-c2ccccc2)nnn1-c1ccc(Br)cc1]

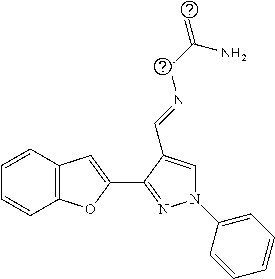 [SMILES: N=C(N)N/N=C/c1cn(-c2ccccc2)nc1-c1cc2ccccc2o1]

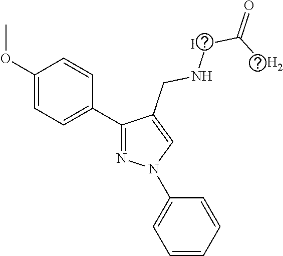 [SMILES: COc1ccc(-c2nn(-c3ccccc3)cc2CNNC(N)=O)cc1]

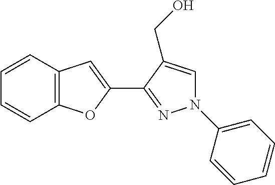 [SMILES: OCc1cn(-c2ccccc2)nc1-c1cc2ccccc2o1]

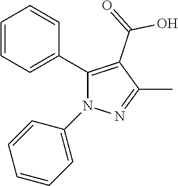 [SMILES: Cc1nn(-c2ccccc2)c(-c2ccccc2)c1C(=O)O]

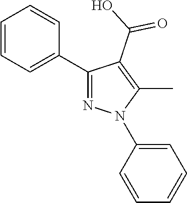 [SMILES: Cc1c(C(=O)O)c(-c2ccccc2)nn1-c1ccccc1]

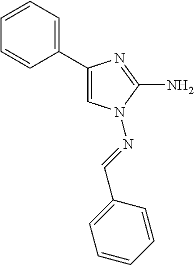 [SMILES: Nc1nc(-c2ccccc2)cn1/N=C/c1ccccc1]

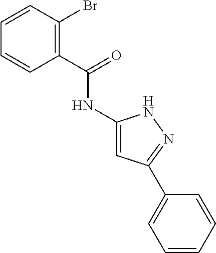 [SMILES: O=C(Nc1cc(-c2ccccc2)n[nH]1)c1ccccc1Br]

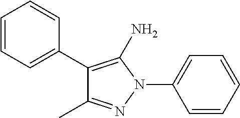 [SMILES: Cc1nn(-c2ccccc2)c(N)c1-c1ccccc1]

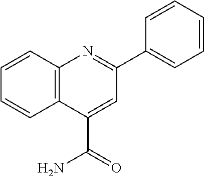 [SMILES: NC(=O)c1cc(-c2ccccc2)nc2ccccc12]

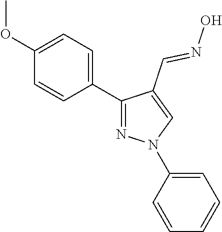 [SMILES: COc1ccc(-c2nn(-c3ccccc3)cc2/C=N/O)cc1]

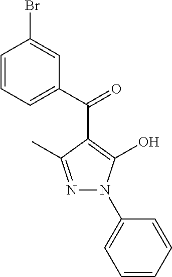 [SMILES: Cc1nn(-c2ccccc2)c(O)c1C(=O)c1cccc(Br)c1]

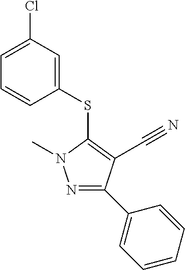 [SMILES: Cn1nc(-c2ccccc2)c(C#N)c1Sc1cccc(Cl)c1]

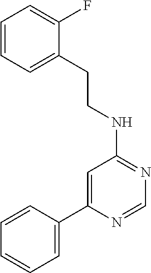 [SMILES: Fc1ccccc1CCNc1cc(-c2ccccc2)ncn1]

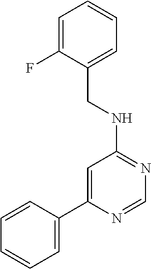 [SMILES: Fc1ccccc1CNc1cc(-c2ccccc2)ncn1]

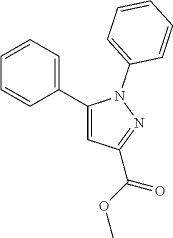 [SMILES: COC(=O)c1cc(-c2ccccc2)n(-c2ccccc2)n1]

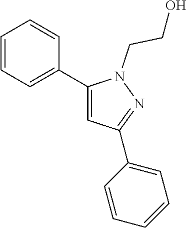 [SMILES: OCCn1nc(-c2ccccc2)cc1-c1ccccc1]

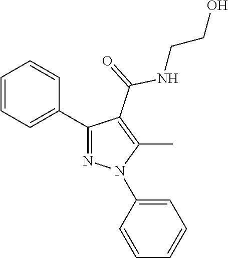 [SMILES: Cc1c(C(=O)NCCO)c(-c2ccccc2)nn1-c1ccccc1]

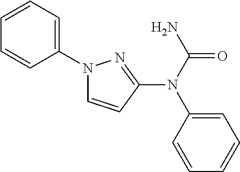 [SMILES: NC(=O)N(c1ccccc1)c1ccn(-c2ccccc2)n1]

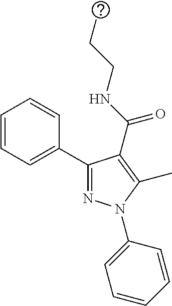 [SMILES: Cc1c(C(=O)NCCBr)c(-c2ccccc2)nn1-c1ccccc1]

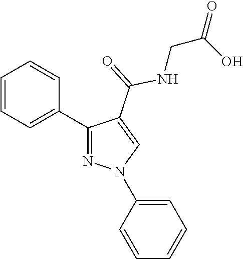 [SMILES: O=C(O)CNC(=O)c1cn(-c2ccccc2)nc1-c1ccccc1]

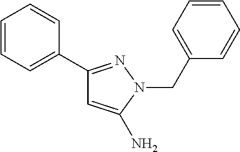 [SMILES: Nc1cc(-c2ccccc2)nn1Cc1ccccc1]

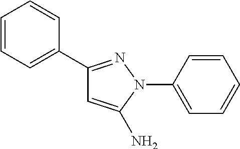 [SMILES: Nc1cc(-c2ccccc2)nn1-c1ccccc1]

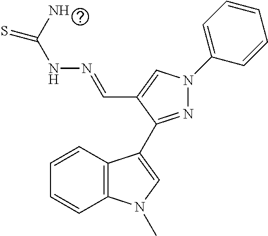 [SMILES: Cn1cc(-c2nn(-c3ccccc3)cc2/C=N/NC(N)=S)c2ccccc21]

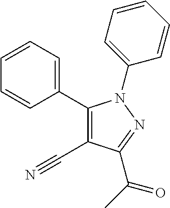 [SMILES: CC(=O)c1nn(-c2ccccc2)c(-c2ccccc2)c1C#N]

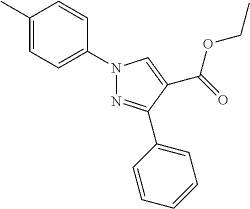 [SMILES: CCOC(=O)c1cn(-c2ccc(C)cc2)nc1-c1ccccc1]

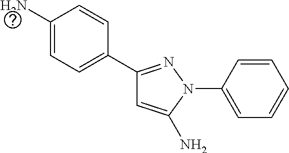 [SMILES: Nc1ccc(-c2cc(N)n(-c3ccccc3)n2)cc1]

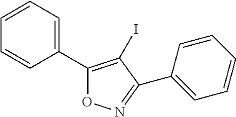 [SMILES: Ic1c(-c2ccccc2)noc1-c1ccccc1]

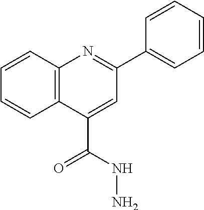 [SMILES: NNC(=O)c1cc(-c2ccccc2)nc2ccccc12]

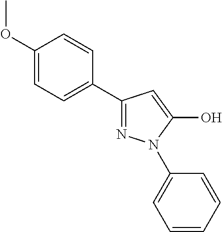 [SMILES: COc1ccc(-c2cc(O)n(-c3ccccc3)n2)cc1]

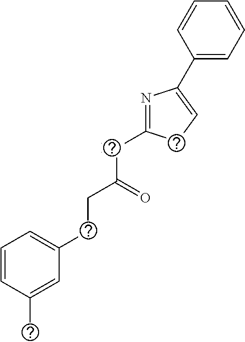 [SMILES: O=C(COc1cccc(Br)c1)Nc1nc(-c2ccccc2)cs1]

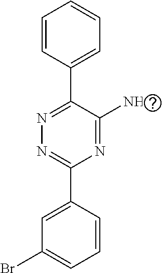 [SMILES: Nc1nc(-c2cccc(Br)c2)nnc1-c1ccccc1]

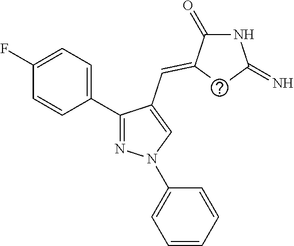 [SMILES: N=C1NC(=O)/C(=C/c2cn(-c3ccccc3)nc2-c2ccc(F)cc2)S1]

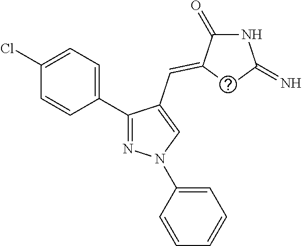 [SMILES: N=C1NC(=O)/C(=C/c2cn(-c3ccccc3)nc2-c2ccc(Cl)cc2)S1]

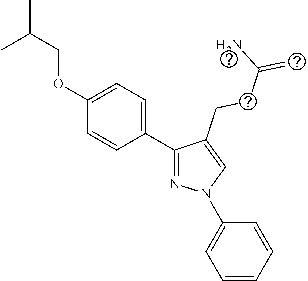 [SMILES: CC(C)COc1ccc(-c2nn(-c3ccccc3)cc2CSC(=N)N)cc1]

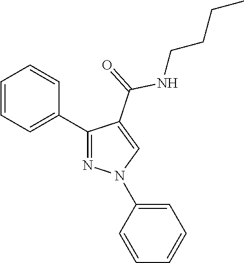 [SMILES: CCCCNC(=O)c1cn(-c2ccccc2)nc1-c1ccccc1]

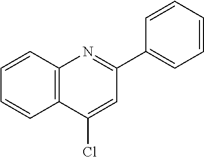 [SMILES: Clc1cc(-c2ccccc2)nc2ccccc12]

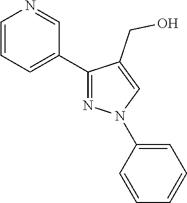 [SMILES: OCc1cn(-c2ccccc2)nc1-c1cccnc1]

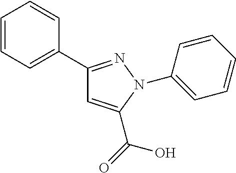 [SMILES: O=C(O)c1cc(-c2ccccc2)nn1-c1ccccc1]

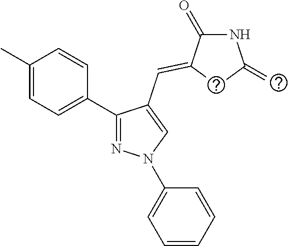 [SMILES: Cc1ccc(-c2nn(-c3ccccc3)cc2/C=C2\SC(=N)NC2=O)cc1]

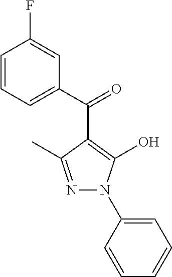 [SMILES: Cc1nn(-c2ccccc2)c(O)c1C(=O)c1cccc(F)c1]

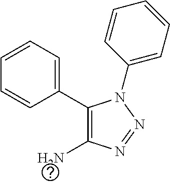 [SMILES: Nc1nnn(-c2ccccc2)c1-c1ccccc1]

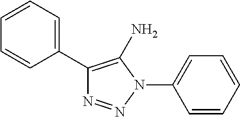 [SMILES: Nc1c(-c2ccccc2)nnn1-c1ccccc1]

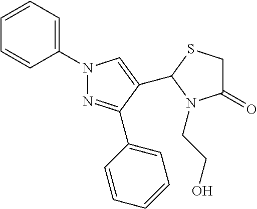 [SMILES: O=C1CSC(c2cn(-c3ccccc3)nc2-c2ccccc2)N1CCO]

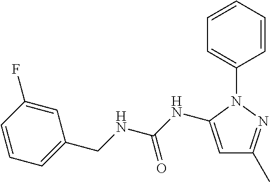 [SMILES: Cc1cc(NC(=O)NCc2cccc(F)c2)n(-c2ccccc2)n1]

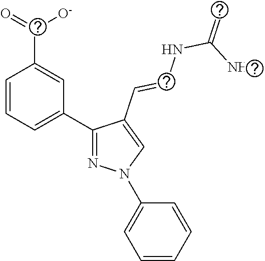 [SMILES: NC(=S)N/N=C/c1cn(-c2ccccc2)nc1-c1cccc([N+](=O)[O-])c1]

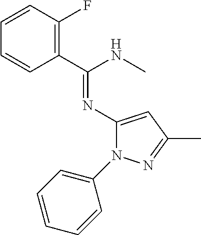 [SMILES: CN/C(=N\c1cc(C)nn1-c1ccccc1)c1ccccc1F]

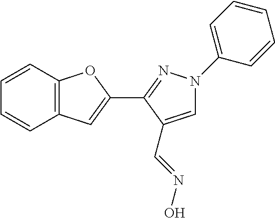 [SMILES: O/N=C/c1cn(-c2ccccc2)nc1-c1cc2ccccc2o1]

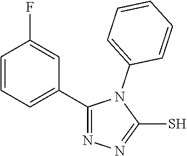 [SMILES: Fc1cccc(-c2nnc(S)n2-c2ccccc2)c1]

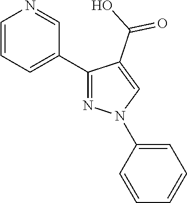 [SMILES: O=C(O)c1cn(-c2ccccc2)nc1-c1cccnc1]

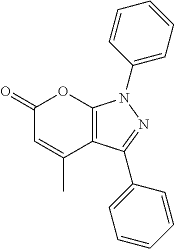 [SMILES: Cc1cc(=O)oc2c1c(-c1ccccc1)nn2-c1ccccc1]

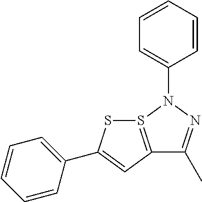 [SMILES: CC1=NN(c2ccccc2)S2=C1C=C(c1ccccc1)S2]

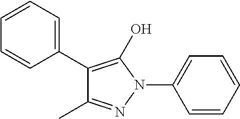 [SMILES: Cc1nn(-c2ccccc2)c(O)c1-c1ccccc1]

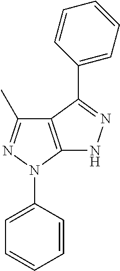 [SMILES: Cc1nn(-c2ccccc2)c2[nH]nc(-c3ccccc3)c12]

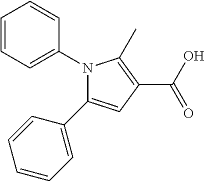 [SMILES: Cc1c(C(=O)O)cc(-c2ccccc2)n1-c1ccccc1]

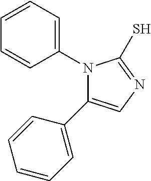 [SMILES: Sc1ncc(-c2ccccc2)n1-c1ccccc1]

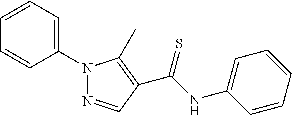 [SMILES: Cc1c(C(=S)Nc2ccccc2)cnn1-c1ccccc1]

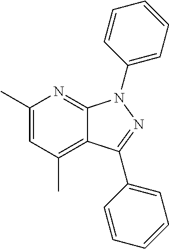 [SMILES: Cc1cc(C)c2c(-c3ccccc3)nn(-c3ccccc3)c2n1]

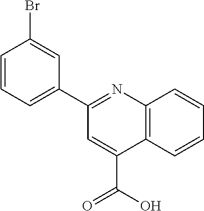 [SMILES: O=C(O)c1cc(-c2cccc(Br)c2)nc2ccccc12]

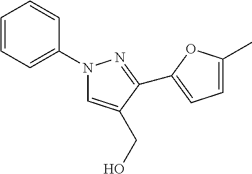 [SMILES: Cc1ccc(-c2nn(-c3ccccc3)cc2CO)o1]

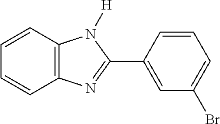 [SMILES: Brc1cccc(-c2nc3ccccc3[nH]2)c1]

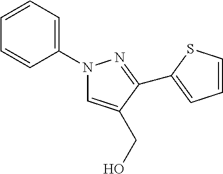 [SMILES: OCc1cn(-c2ccccc2)nc1-c1cccs1]

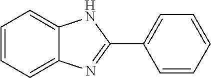 [SMILES: c1ccc(-c2nc3ccccc3[nH]2)cc1]

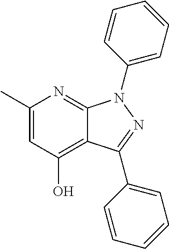 [SMILES: Cc1cc(O)c2c(-c3ccccc3)nn(-c3ccccc3)c2n1]

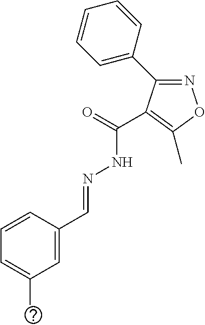 [SMILES: Cc1onc(-c2ccccc2)c1C(=O)N/N=C/c1cccc(Br)c1]

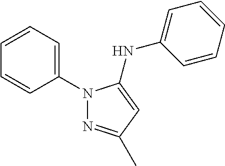 [SMILES: Cc1cc(Nc2ccccc2)n(-c2ccccc2)n1]

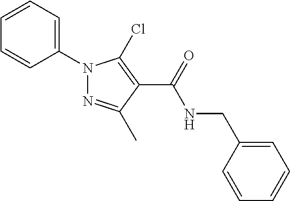 [SMILES: Cc1nn(-c2ccccc2)c(Cl)c1C(=O)NCc1ccccc1]

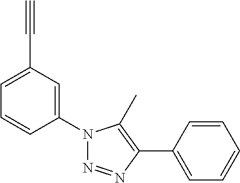 [SMILES: C#Cc1cccc(-n2nnc(-c3ccccc3)c2C)c1]

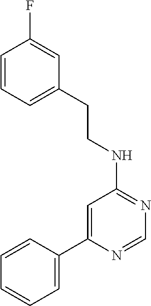 [SMILES: Fc1cccc(CCNc2cc(-c3ccccc3)ncn2)c1]

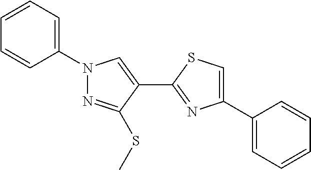 [SMILES: CSc1nn(-c2ccccc2)cc1-c1nc(-c2ccccc2)cs1]

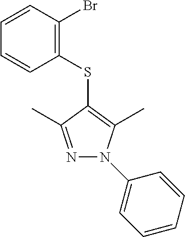 [SMILES: Cc1nn(-c2ccccc2)c(C)c1Sc1ccccc1Br]

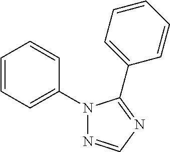 [SMILES: c1ccc(-c2ncnn2-c2ccccc2)cc1]

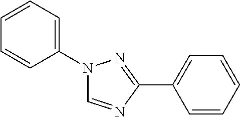 [SMILES: c1ccc(-c2ncn(-c3ccccc3)n2)cc1]

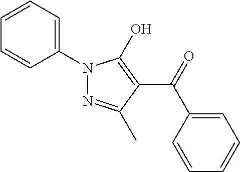 [SMILES: Cc1nn(-c2ccccc2)c(O)c1C(=O)c1ccccc1]

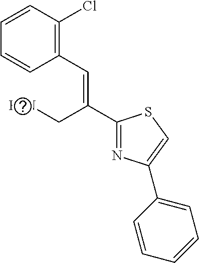 [SMILES: NC/C(=C\c1ccccc1Cl)c1nc(-c2ccccc2)cs1]

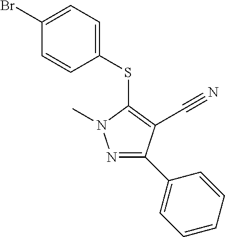 [SMILES: Cn1nc(-c2ccccc2)c(C#N)c1Sc1ccc(Br)cc1]

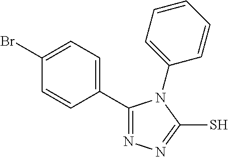 [SMILES: Sc1nnc(-c2ccc(Br)cc2)n1-c1ccccc1]

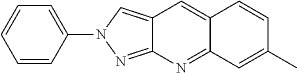 [SMILES: Cc1ccc2cc3cn(-c4ccccc4)nc3nc2c1]

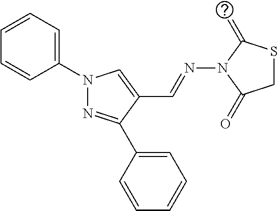 [SMILES: O=C1CSC(=S)N1/N=C/c1cn(-c2ccccc2)nc1-c1ccccc1]

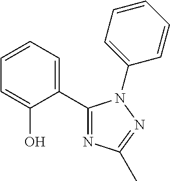 [SMILES: Cc1nc(-c2ccccc2O)n(-c2ccccc2)n1]

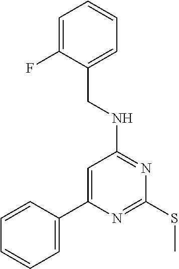 [SMILES: CSc1nc(NCc2ccccc2F)cc(-c2ccccc2)n1]

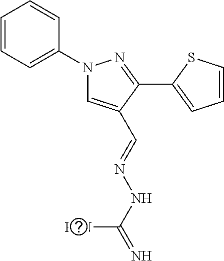 [SMILES: N=C(N)N/N=C/c1cn(-c2ccccc2)nc1-c1cccs1]

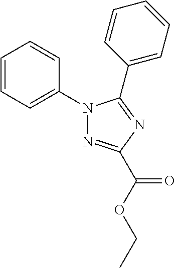 [SMILES: CCOC(=O)c1nc(-c2ccccc2)n(-c2ccccc2)n1]

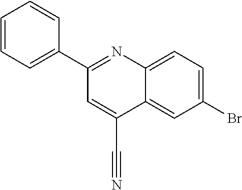 [SMILES: N#Cc1cc(-c2ccccc2)nc2ccc(Br)cc12]

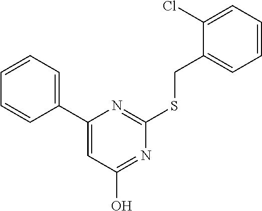 [SMILES: Oc1cc(-c2ccccc2)nc(SCc2ccccc2Cl)n1]

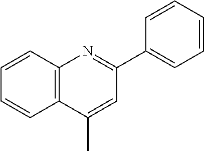 [SMILES: Cc1cc(-c2ccccc2)nc2ccccc12]

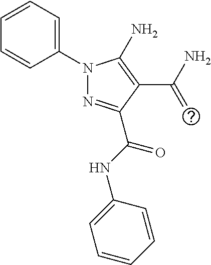 [SMILES: NC(=S)c1c(C(=O)Nc2ccccc2)nn(-c2ccccc2)c1N]

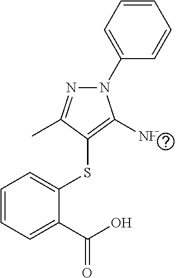 [SMILES: Cc1nn(-c2ccccc2)c(N)c1Sc1ccccc1C(=O)O]

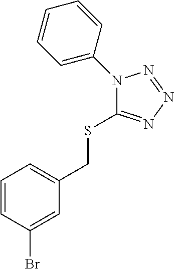 [SMILES: Brc1cccc(CSc2nnnn2-c2ccccc2)c1]

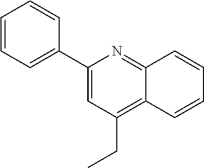 [SMILES: CCc1cc(-c2ccccc2)nc2ccccc12]

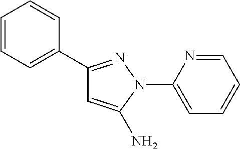 [SMILES: Nc1cc(-c2ccccc2)nn1-c1ccccn1]

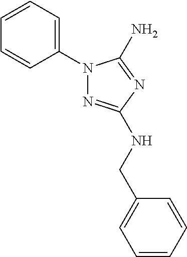 [SMILES: Nc1nc(NCc2ccccc2)nn1-c1ccccc1]

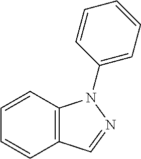 [SMILES: c1ccc(-n2ncc3ccccc32)cc1]

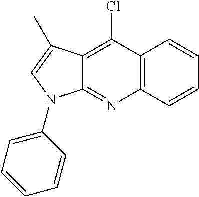 [SMILES: Cc1cn(-c2ccccc2)c2nc3ccccc3c(Cl)c12]

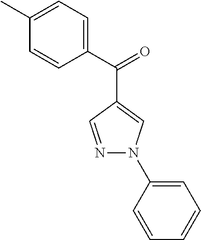 [SMILES: Cc1ccc(C(=O)c2cnn(-c3ccccc3)c2)cc1]

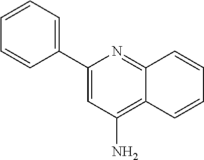 [SMILES: Nc1cc(-c2ccccc2)nc2ccccc12]

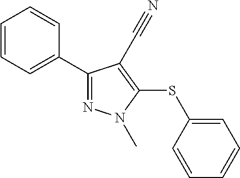 [SMILES: Cn1nc(-c2ccccc2)c(C#N)c1Sc1ccccc1]

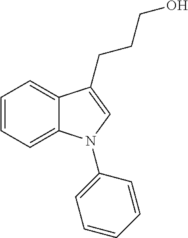 [SMILES: OCCCc1cn(-c2ccccc2)c2ccccc12]

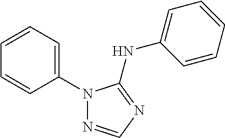 [SMILES: c1ccc(Nc2ncnn2-c2ccccc2)cc1]

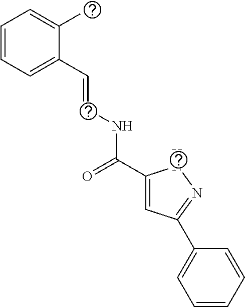 [SMILES: O=C(N/N=C/c1ccccc1Br)c1cc(-c2ccccc2)n[nH]1]

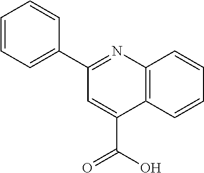 [SMILES: O=C(O)c1cc(-c2ccccc2)nc2ccccc12]

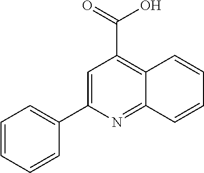 [SMILES: O=C(O)c1cc(-c2ccccc2)nc2ccccc12]

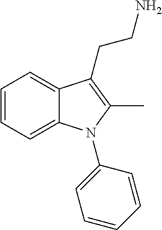 [SMILES: Cc1c(CCN)c2ccccc2n1-c1ccccc1]

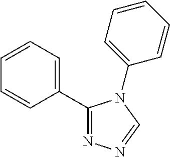 [SMILES: c1ccc(-c2nncn2-c2ccccc2)cc1]

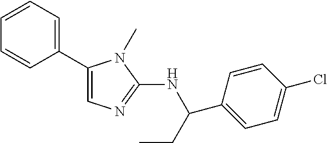 [SMILES: CCC(Nc1ncc(-c2ccccc2)n1C)c1ccc(Cl)cc1]

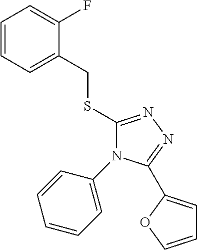 [SMILES: Fc1ccccc1CSc1nnc(-c2ccco2)n1-c1ccccc1]

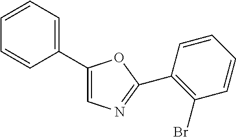 [SMILES: Brc1ccccc1-c1ncc(-c2ccccc2)o1]

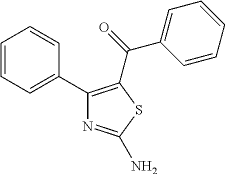 [SMILES: Nc1nc(-c2ccccc2)c(C(=O)c2ccccc2)s1]

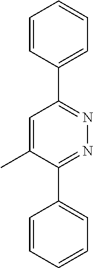 [SMILES: Cc1cc(-c2ccccc2)nnc1-c1ccccc1]

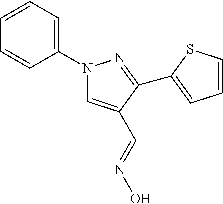 [SMILES: O/N=C/c1cn(-c2ccccc2)nc1-c1cccs1]

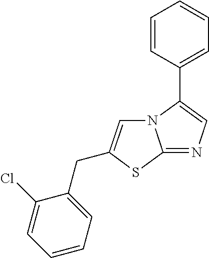 [SMILES: Clc1ccccc1Cc1cn2c(-c3ccccc3)cnc2s1]

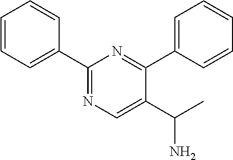 [SMILES: CC(N)c1cnc(-c2ccccc2)nc1-c1ccccc1]

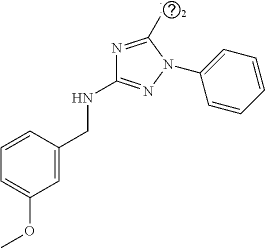 [SMILES: COc1cccc(CNc2nc(N)n(-c3ccccc3)n2)c1]

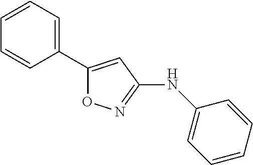 [SMILES: c1ccc(Nc2cc(-c3ccccc3)on2)cc1]

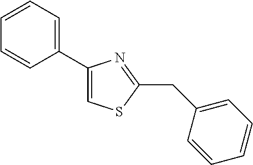 [SMILES: c1ccc(Cc2nc(-c3ccccc3)cs2)cc1]

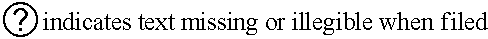

TABLE 7
Compounds that contain the diphenylpyazole substructure
present in Formula Ia.

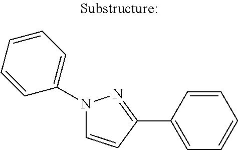 [SMILES: c1ccc(-c2ccn(-c3ccccc3)n2)cc1]

Structures

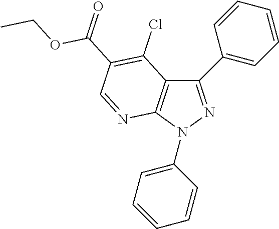 [SMILES: CCOC(=O)c1cnc2c(c(-c3ccccc3)nn2-c2ccccc2)c1Cl]

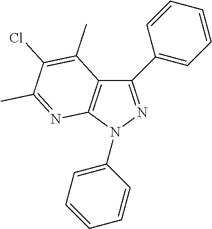 [SMILES: Cc1nc2c(c(-c3ccccc3)nn2-c2ccccc2)c(C)c1Cl]

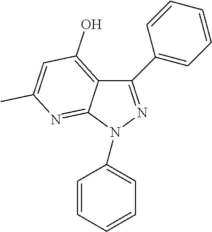 [SMILES: Cc1cc(O)c2c(-c3ccccc3)nn(-c3ccccc3)c2n1]

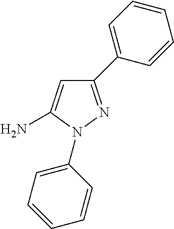 [SMILES: Nc1cc(-c2ccccc2)nn1-c1ccccc1]

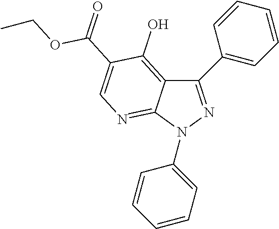 [SMILES: CCOC(=O)c1cnc2c(c(-c3ccccc3)nn2-c2ccccc2)c1O]

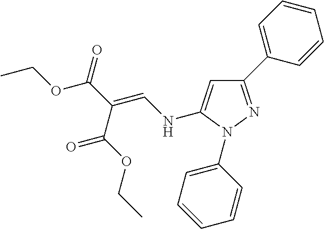 [SMILES: CCOC(=O)C(=CNc1cc(-c2ccccc2)nn1-c1ccccc1)C(=O)OCC]

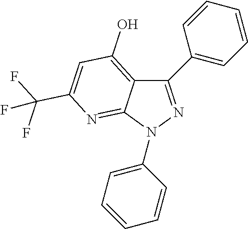 [SMILES: Oc1cc(C(F)(F)F)nc2c1c(-c1ccccc1)nn2-c1ccccc1]

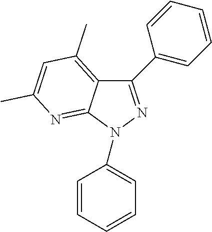 [SMILES: Cc1cc(C)c2c(-c3ccccc3)nn(-c3ccccc3)c2n1]

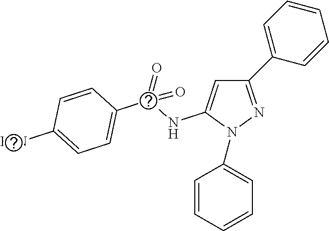 [SMILES: Nc1ccc(S(=O)(=O)Nc2cc(-c3ccccc3)nn2-c2ccccc2)cc1]

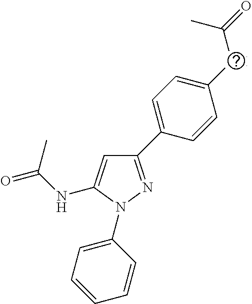 [SMILES: CC(=O)Nc1ccc(-c2cc(NC(C)=O)n(-c3ccccc3)n2)cc1]

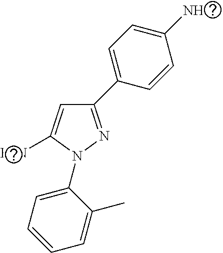 [SMILES: Cc1ccccc1-n1nc(-c2ccc(N)cc2)cc1N]

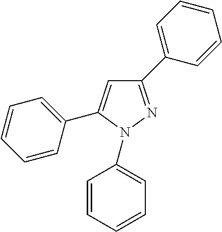 [SMILES: c1ccc(-c2cc(-c3ccccc3)n(-c3ccccc3)n2)cc1]

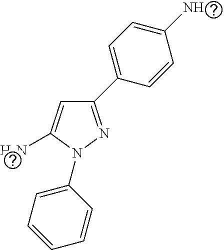 [SMILES: Nc1ccc(-c2cc(N)n(-c3ccccc3)n2)cc1]

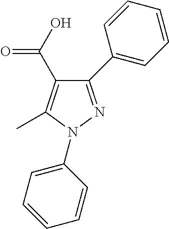 [SMILES: Cc1c(C(=O)O)c(-c2ccccc2)nn1-c1ccccc1]

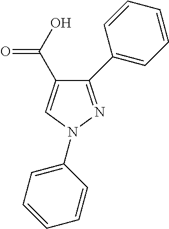 [SMILES: O=C(O)c1cn(-c2ccccc2)nc1-c1ccccc1]

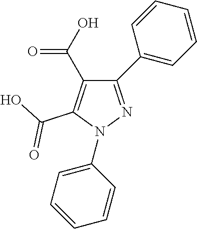 [SMILES: O=C(O)c1c(-c2ccccc2)nn(-c2ccccc2)c1C(=O)O]

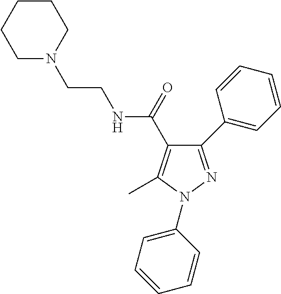 [SMILES: Cc1c(C(=O)NCCN2CCCCC2)c(-c2ccccc2)nn1-c1ccccc1]

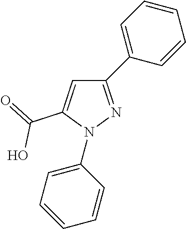 [SMILES: O=C(O)c1cc(-c2ccccc2)nn1-c1ccccc1]

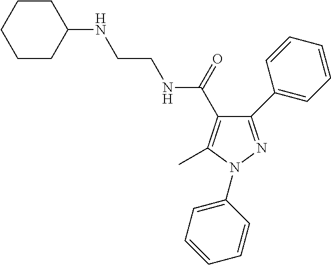 [SMILES: Cc1c(C(=O)NCCNC2CCCCC2)c(-c2ccccc2)nn1-c1ccccc1]

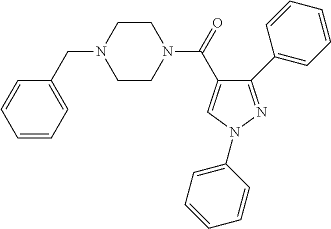 [SMILES: O=C(c1cn(-c2ccccc2)nc1-c1ccccc1)N1CCN(Cc2ccccc2)CC1]

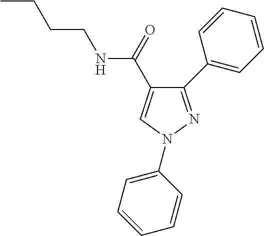 [SMILES: CCCCNC(=O)c1cn(-c2ccccc2)nc1-c1ccccc1]

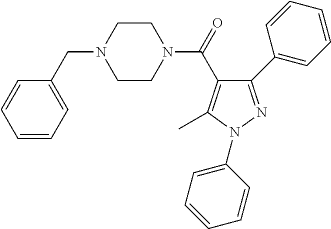 [SMILES: Cc1c(C(=O)N2CCN(Cc3ccccc3)CC2)c(-c2ccccc2)nn1-c1ccccc1]

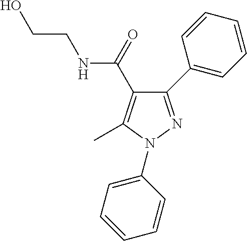 [SMILES: Cc1c(C(=O)NCCO)c(-c2ccccc2)nn1-c1ccccc1]

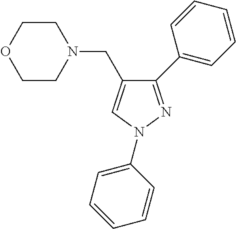 [SMILES: c1ccc(-c2nn(-c3ccccc3)cc2CN2CCOCC2)cc1]

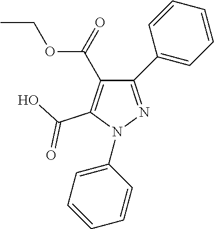 [SMILES: CCOC(=O)c1c(-c2ccccc2)nn(-c2ccccc2)c1C(=O)O]

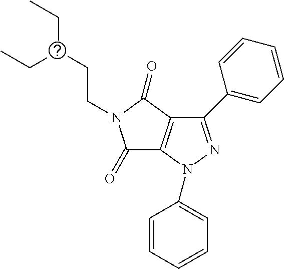 [SMILES: CCN(CC)CCN1C(=O)c2c(-c3ccccc3)nn(-c3ccccc3)c2C1=O]

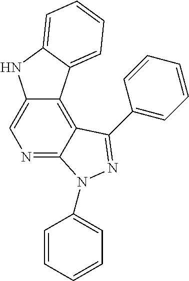 [SMILES: c1ccc(-c2nn(-c3ccccc3)c3ncc4[nH]c5ccccc5c4c23)cc1]

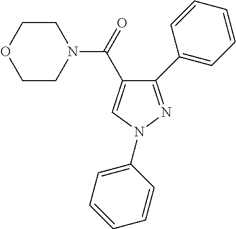 [SMILES: O=C(c1cn(-c2ccccc2)nc1-c1ccccc1)N1CCOCC1]

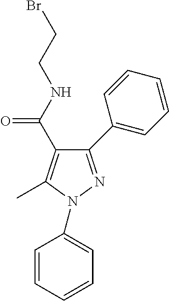 [SMILES: Cc1c(C(=O)NCCBr)c(-c2ccccc2)nn1-c1ccccc1]

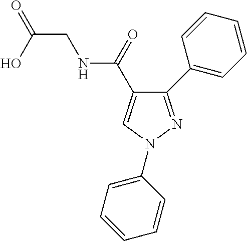 [SMILES: O=C(O)CNC(=O)c1cn(-c2ccccc2)nc1-c1ccccc1]

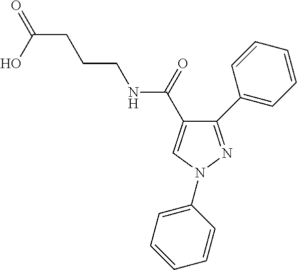 [SMILES: O=C(O)CCCNC(=O)c1cn(-c2ccccc2)nc1-c1ccccc1]

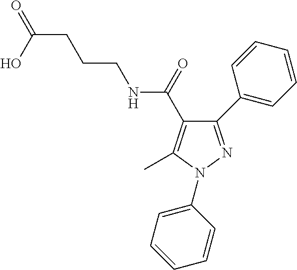 [SMILES: Cc1c(C(=O)NCCCC(=O)O)c(-c2ccccc2)nn1-c1ccccc1]

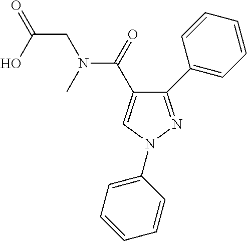 [SMILES: CN(CC(=O)O)C(=O)c1cn(-c2ccccc2)nc1-c1ccccc1]

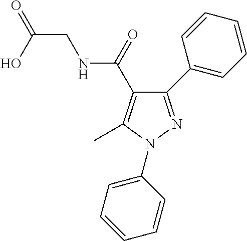 [SMILES: Cc1c(C(=O)NCC(=O)O)c(-c2ccccc2)nn1-c1ccccc1]

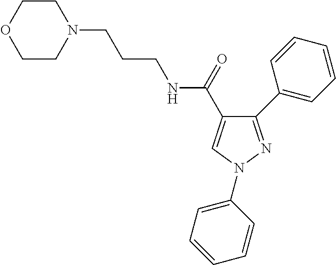 [SMILES: O=C(NCCCN1CCOCC1)c1cn(-c2ccccc2)nc1-c1ccccc1]

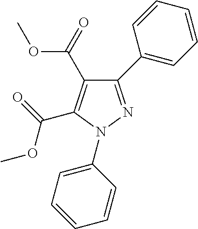 [SMILES: COC(=O)c1c(-c2ccccc2)nn(-c2ccccc2)c1C(=O)OC]

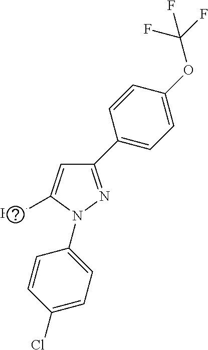 [SMILES: Nc1cc(-c2ccc(OC(F)(F)F)cc2)nn1-c1ccc(Cl)cc1]

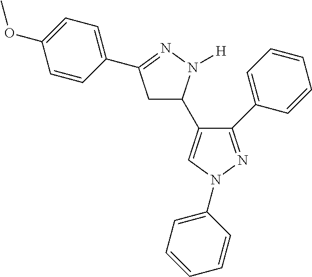 [SMILES: COc1ccc(C2=NNC(c3cn(-c4ccccc4)nc3-c3ccccc3)C2)cc1]

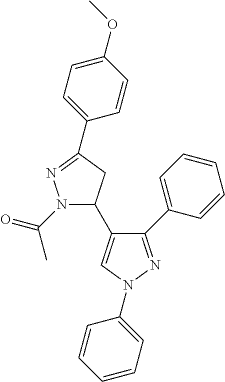 [SMILES: COc1ccc(C2=NN(C(C)=O)C(c3cn(-c4ccccc4)nc3-c3ccccc3)C2)cc1]

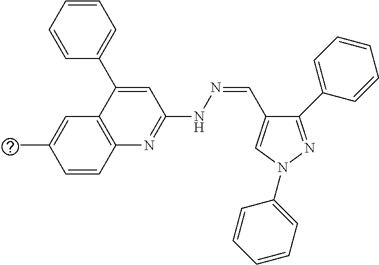 [SMILES: Brc1ccc2nc(N/N=C\c3cn(-c4ccccc4)nc3-c3ccccc3)cc(-c3ccccc3)c2c1]

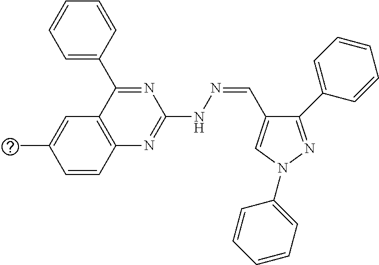 [SMILES: Brc1ccc2nc(N/N=C\c3cn(-c4ccccc4)nc3-c3ccccc3)nc(-c3ccccc3)c2c1]

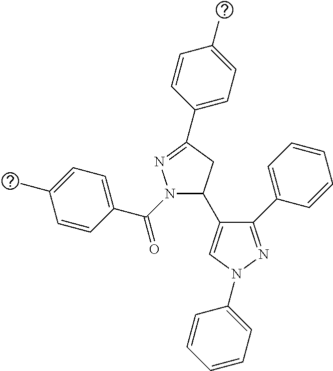 [SMILES: O=C(c1ccc(Cl)cc1)N1N=C(c2ccc(Br)cc2)CC1c1cn(-c2ccccc2)nc1-c1ccccc1]

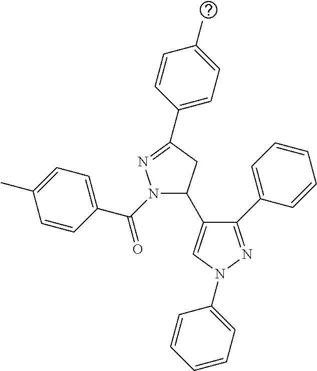 [SMILES: Cc1ccc(C(=O)N2N=C(c3ccc(Br)cc3)CC2c2cn(-c3ccccc3)nc2-c2ccccc2)cc1]

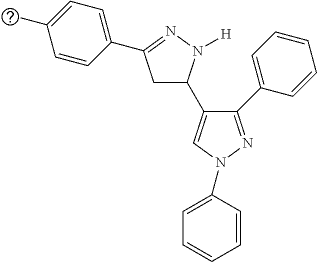 [SMILES: Brc1ccc(C2=NNC(c3cn(-c4ccccc4)nc3-c3ccccc3)C2)cc1]

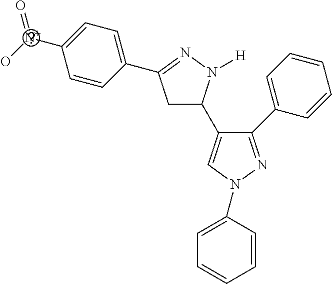

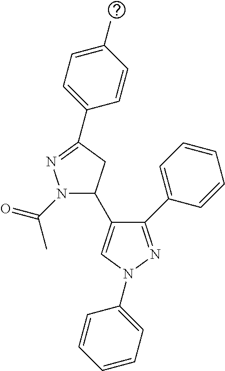 [SMILES: CC(=O)N1N=C(c2ccc(Br)cc2)CC1c1cn(-c2ccccc2)nc1-c1ccccc1]

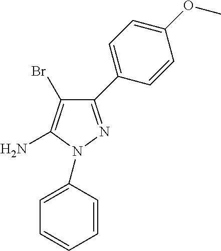 [SMILES: COc1ccc(-c2nn(-c3ccccc3)c(N)c2Br)cc1]

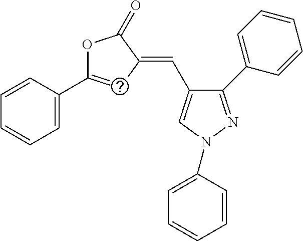

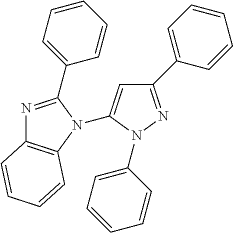 [SMILES: c1ccc(-c2cc(-n3c(-c4ccccc4)nc4ccccc43)n(-c3ccccc3)n2)cc1]

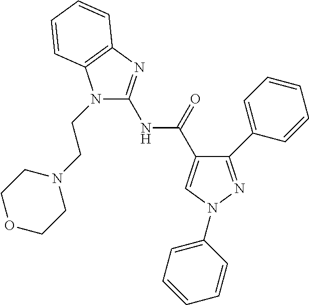 [SMILES: O=C(Nc1nc2ccccc2n1CCN1CCOCC1)c1cn(-c2ccccc2)nc1-c1ccccc1]

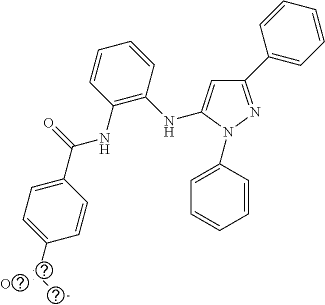 [SMILES: O=C(Nc1ccccc1Nc1cc(-c2ccccc2)nn1-c1ccccc1)c1ccc([NH+]([O-])O)cc1]

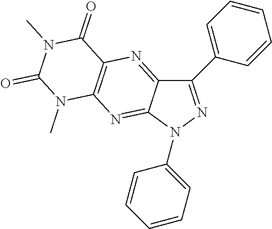 [SMILES: Cn1c(=O)c2nc3c(-c4ccccc4)nn(-c4ccccc4)c3nc2n(C)c1=O]

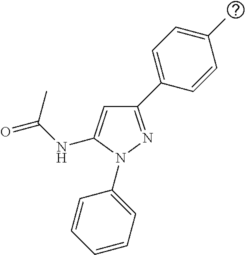 [SMILES: CC(=O)Nc1cc(-c2ccc(Br)cc2)nn1-c1ccccc1]

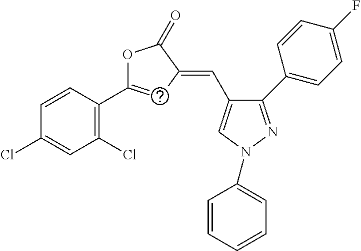

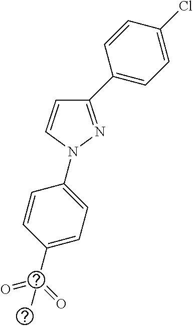 [SMILES: O=S(=O)(Cl)c1ccc(-n2ccc(-c3ccc(Cl)cc3)n2)cc1]

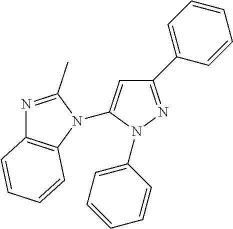 [SMILES: Cc1nc2ccccc2n1-c1cc(-c2ccccc2)nn1-c1ccccc1]

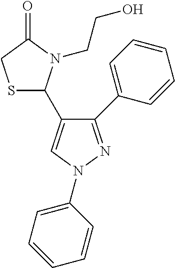 [SMILES: O=C1CSC(c2cn(-c3ccccc3)nc2-c2ccccc2)N1CCO]

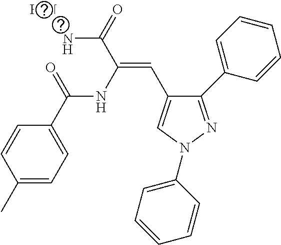 [SMILES: Cc1ccc(C(=O)N/C(=C\c2cn(-c3ccccc3)nc2-c2ccccc2)C(=O)NN)cc1]

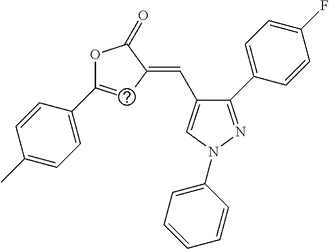 [SMILES: Cc1ccc(C2=N/C(=C\c3cn(-c4ccccc4)nc3-c3ccc(F)cc3)C(=O)O2)cc1]

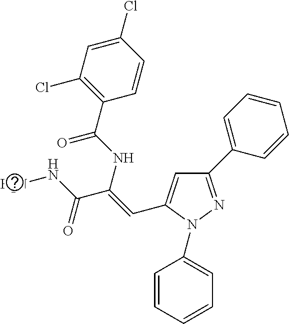 [SMILES: NNC(=O)/C(=C/c1cc(-c2ccccc2)nn1-c1ccccc1)NC(=O)c1ccc(Cl)cc1Cl]

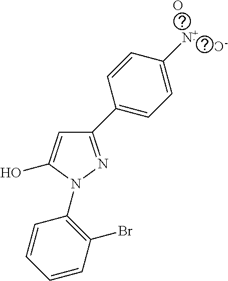 [SMILES: [O-][NH+](O)c1ccc(-c2cc(O)n(-c3ccccc3Br)n2)cc1]

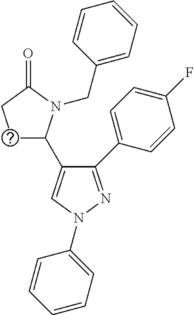 [SMILES: O=C1CSC(c2cn(-c3ccccc3)nc2-c2ccc(F)cc2)N1Cc1ccccc1]

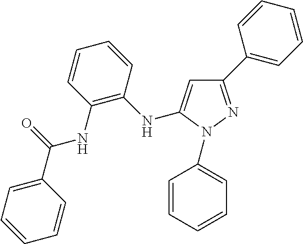 [SMILES: O=C(Nc1ccccc1Nc1cc(-c2ccccc2)nn1-c1ccccc1)c1ccccc1]

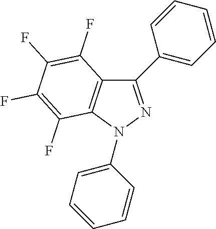 [SMILES: Fc1c(F)c(F)c2c(c(-c3ccccc3)nn2-c2ccccc2)c1F]

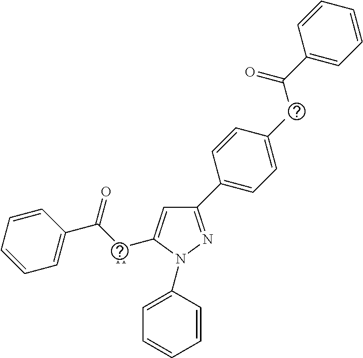 [SMILES: O=C(Nc1ccc(-c2cc(NC(=O)c3ccccc3)n(-c3ccccc3)n2)cc1)c1ccccc1]

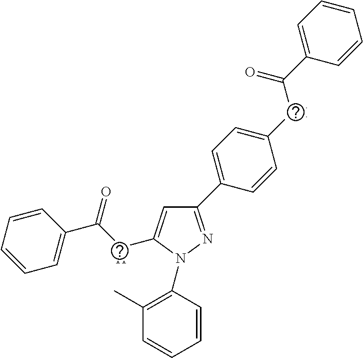 [SMILES: Cc1ccccc1-n1nc(-c2ccc(NC(=O)c3ccccc3)cc2)cc1NC(=O)c1ccccc1]

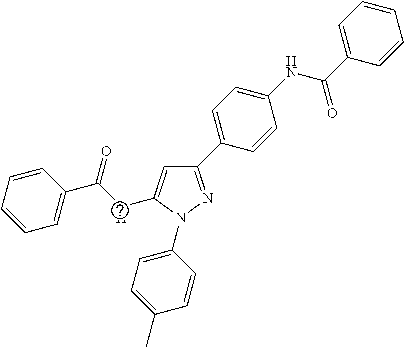 [SMILES: Cc1ccc(-n2nc(-c3ccc(NC(=O)c4ccccc4)cc3)cc2NC(=O)c2ccccc2)cc1]

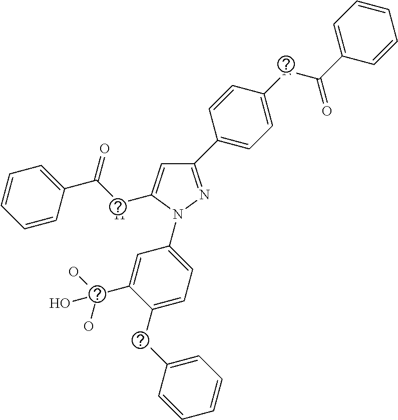

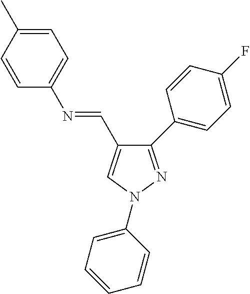 [SMILES: Cc1ccc(/N=C/c2cn(-c3ccccc3)nc2-c2ccc(F)cc2)cc1]

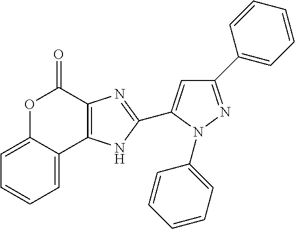 [SMILES: O=c1oc2ccccc2c2[nH]c(-c3cc(-c4ccccc4)nn3-c3ccccc3)nc12]

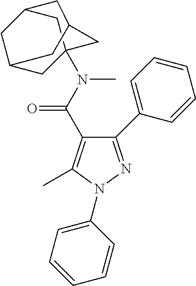 [SMILES: Cc1c(C(=O)N(C)C23CC4CC(CC(C4)C2)C3)c(-c2ccccc2)nn1-c1ccccc1]

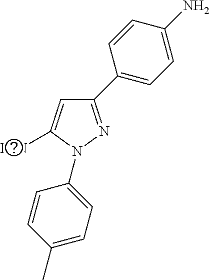 [SMILES: Cc1ccc(-n2nc(-c3ccc(N)cc3)cc2N)cc1]

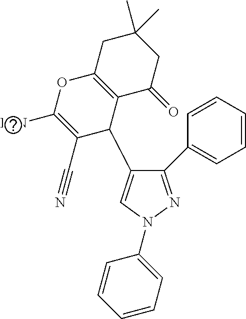 [SMILES: CC1(C)CC(=O)C2=C(C1)OC(N)=C(C#N)C2c1cn(-c2ccccc2)nc1-c1ccccc1]

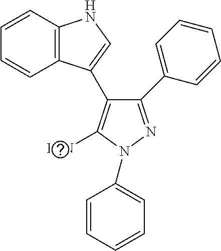 [SMILES: Nc1c(-c2c[nH]c3ccccc23)c(-c2ccccc2)nn1-c1ccccc1]

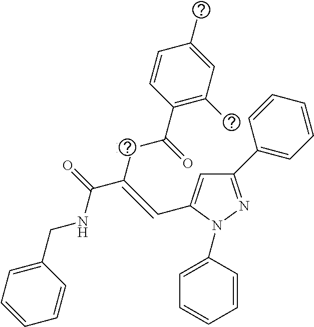 [SMILES: O=C(NCc1ccccc1)/C(=C/c1cc(-c2ccccc2)nn1-c1ccccc1)NC(=O)c1ccc(Cl)cc1Cl]

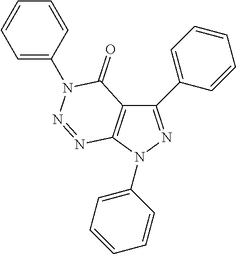 [SMILES: O=c1c2c(-c3ccccc3)nn(-c3ccccc3)c2nnn1-c1ccccc1]

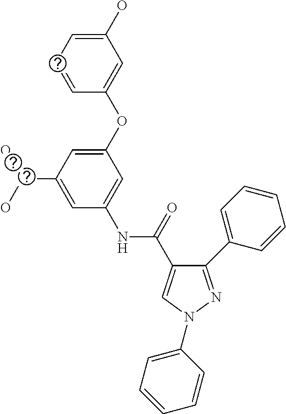

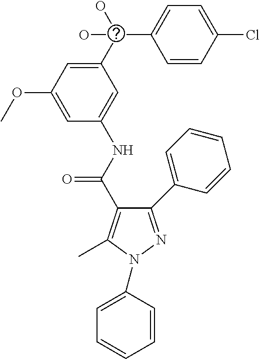 [SMILES: COc1cc(NC(=O)c2c(-c3ccccc3)nn(-c3ccccc3)c2C)cc(S(O)(O)c2ccc(Cl)cc2)c1]

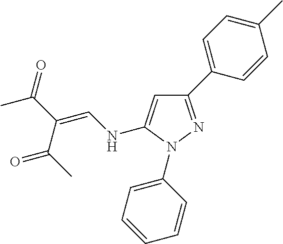 [SMILES: CC(=O)C(=CNc1cc(-c2ccc(C)cc2)nn1-c1ccccc1)C(C)=O]

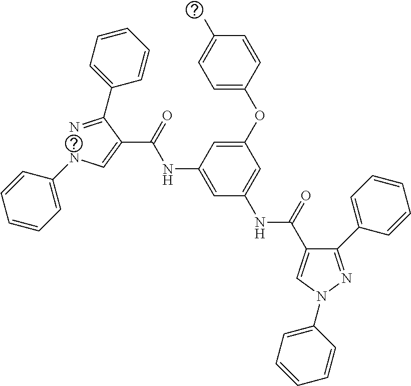 [SMILES: O=C(Nc1cc(NC(=O)c2cn(-c3ccccc3)nc2-c2ccccc2)cc(Oc2ccc(Br)cc2)c1)c1cn(-c2ccccc2)nc1-c1ccccc1]

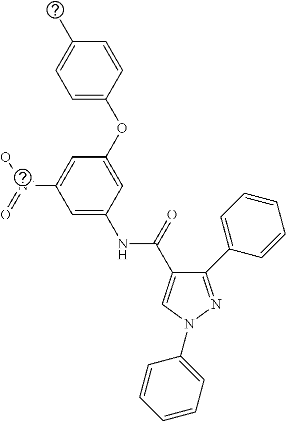

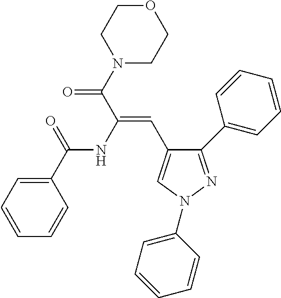 [SMILES: O=C(N/C(=C\c1cn(-c2ccccc2)nc1-c1ccccc1)C(=O)N1CCOCC1)c1ccccc1]

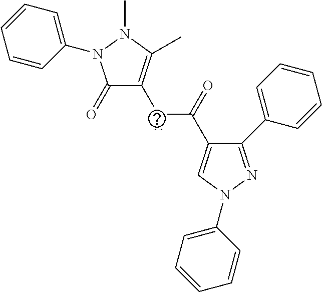 [SMILES: Cc1c(NC(=O)c2cn(-c3ccccc3)nc2-c2ccccc2)c(=O)n(-c2ccccc2)n1C]

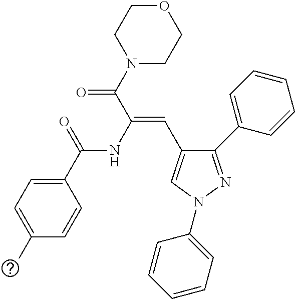 [SMILES: O=C(N/C(=C\c1cn(-c2ccccc2)nc1-c1ccccc1)C(=O)N1CCOCC1)c1ccc(Br)cc1]

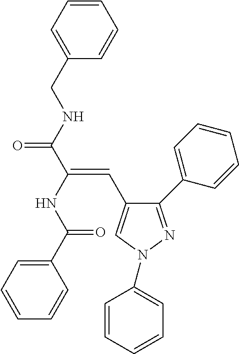 [SMILES: O=C(NCc1ccccc1)/C(=C/c1cn(-c2ccccc2)nc1-c1ccccc1)NC(=O)c1ccccc1]

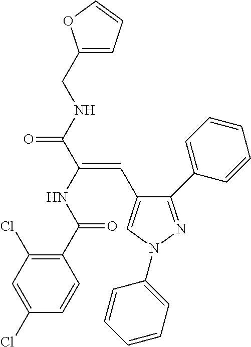 [SMILES: O=C(NCc1ccco1)/C(=C/c1cn(-c2ccccc2)nc1-c1ccccc1)NC(=O)c1ccc(Cl)cc1Cl]

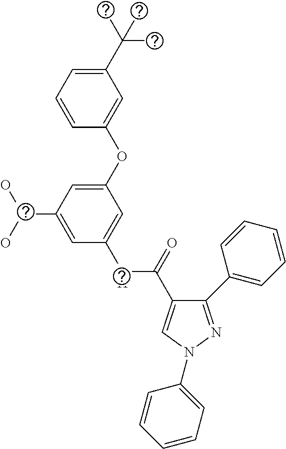 [SMILES: O=C(Nc1cc(Oc2cccc(C(F)(F)F)c2)cc([NH+](O)O)c1)c1cn(-c2ccccc2)nc1-c1ccccc1]

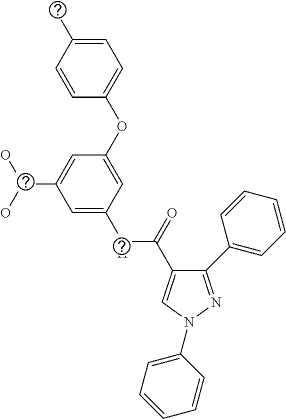 [SMILES: O=C(Nc1cc(Oc2ccc(I)cc2)cc([NH+](O)O)c1)c1cn(-c2ccccc2)nc1-c1ccccc1]

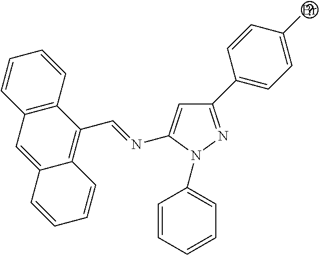 [SMILES: Brc1ccc(-c2cc(/N=C/c3c4ccccc4cc4ccccc34)n(-c3ccccc3)n2)cc1]

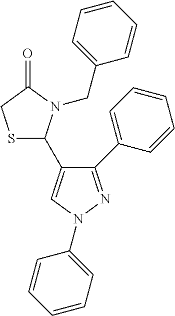 [SMILES: O=C1CSC(c2cn(-c3ccccc3)nc2-c2ccccc2)N1Cc1ccccc1]

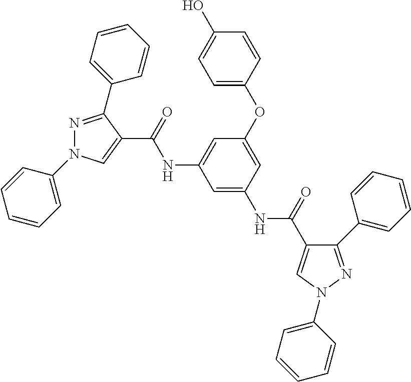 [SMILES: O=C(Nc1cc(NC(=O)c2cn(-c3ccccc3)nc2-c2ccccc2)cc(Oc2ccc(O)cc2)c1)c1cn(-c2ccccc2)nc1-c1ccccc1]

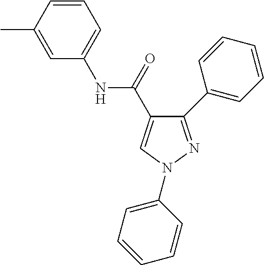 [SMILES: Cc1cccc(NC(=O)c2cn(-c3ccccc3)nc2-c2ccccc2)c1]

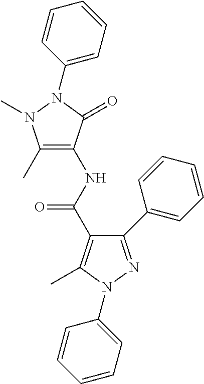 [SMILES: Cc1c(C(=O)Nc2c(C)n(C)n(-c3ccccc3)c2=O)c(-c2ccccc2)nn1-c1ccccc1]

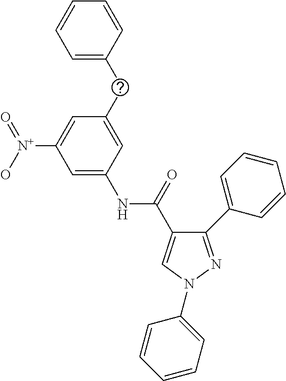

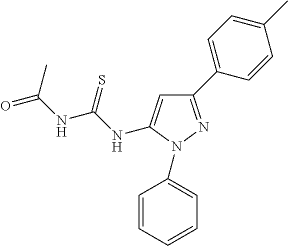 [SMILES: CC(=O)NC(=S)Nc1cc(-c2ccc(C)cc2)nn1-c1ccccc1]

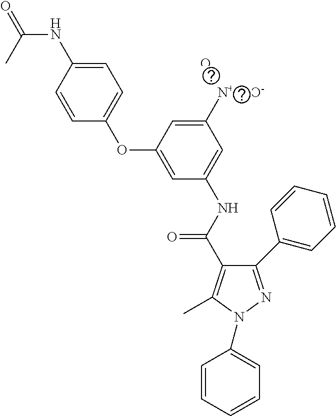 [SMILES: CC(=O)Nc1ccc(Oc2cc(NC(=O)c3c(-c4ccccc4)nn(-c4ccccc4)c3C)cc([NH+]([O-])O)c2)cc1]

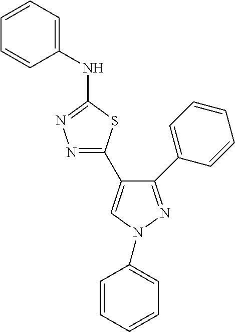 [SMILES: c1ccc(Nc2nnc(-c3cn(-c4ccccc4)nc3-c3ccccc3)s2)cc1]

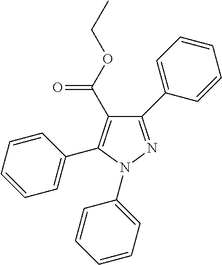 [SMILES: CCOC(=O)c1c(-c2ccccc2)nn(-c2ccccc2)c1-c1ccccc1]

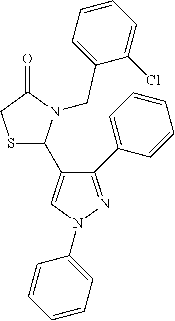 [SMILES: O=C1CSC(c2cn(-c3ccccc3)nc2-c2ccccc2)N1Cc1ccccc1Cl]

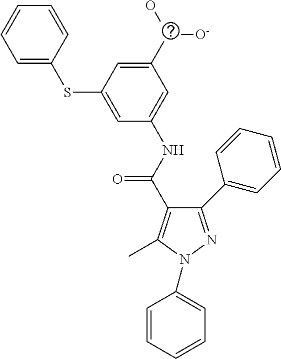 [SMILES: Cc1c(C(=O)Nc2cc(Sc3ccccc3)cc([NH+]([O-])O)c2)c(-c2ccccc2)nn1-c1ccccc1]

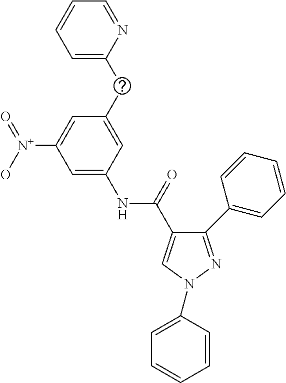

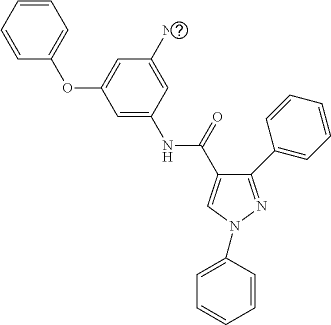 [SMILES: Nc1cc(NC(=O)c2cn(-c3ccccc3)nc2-c2ccccc2)cc(Oc2ccccc2)c1]

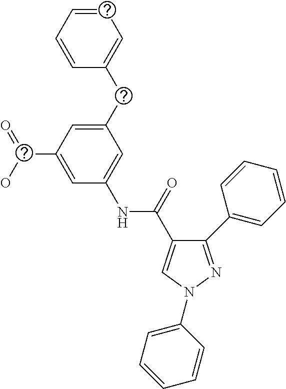

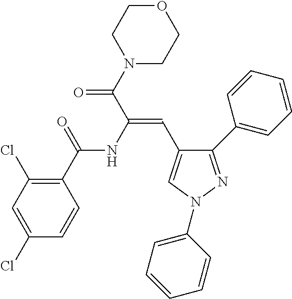 [SMILES: O=C(N/C(=C\c1cn(-c2ccccc2)nc1-c1ccccc1)C(=O)N1CCOCC1)c1ccc(Cl)cc1Cl]

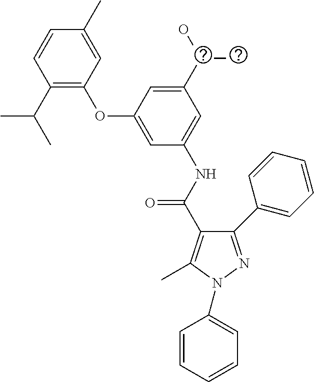 [SMILES: Cc1ccc(C(C)C)c(Oc2cc(NC(=O)c3c(-c4ccccc4)nn(-c4ccccc4)c3C)cc([NH+]([O-])O)c2)c1]

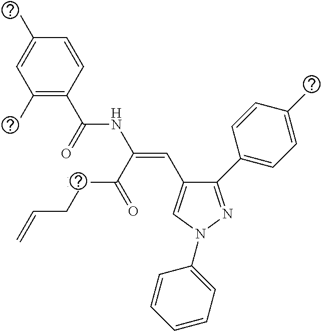 [SMILES: C=CCNC(=O)/C(=C\c1cn(-c2ccccc2)nc1-c1ccc(F)cc1)NC(=O)c1ccc(Cl)cc1Cl]

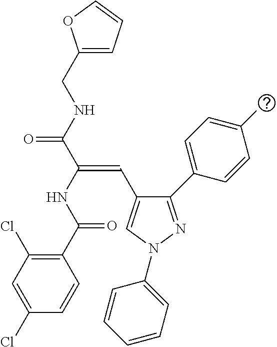 [SMILES: O=C(NCc1ccco1)/C(=C/c1cn(-c2ccccc2)nc1-c1ccc(F)cc1)NC(=O)c1ccc(Cl)cc1Cl]

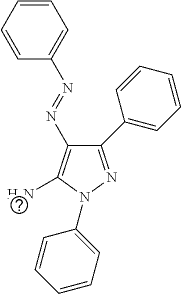 [SMILES: Nc1c(/N=N/c2ccccc2)c(-c2ccccc2)nn1-c1ccccc1]

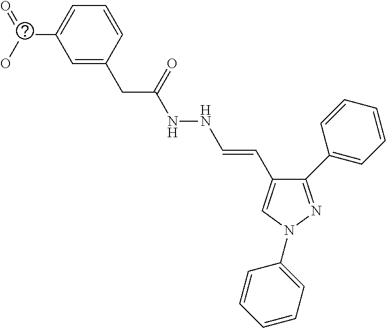

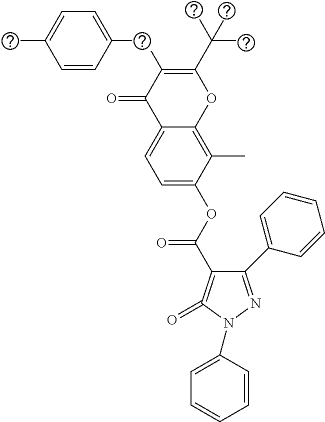

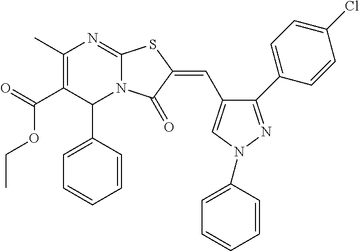 [SMILES: CCOC(=O)C1=C(C)N=c2s/c(=C/c3cn(-c4ccccc4)nc3-c3ccc(Cl)cc3)c(=O)n2C1c1ccccc1]

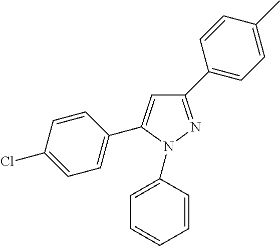 [SMILES: Cc1ccc(-c2cc(-c3ccc(Cl)cc3)n(-c3ccccc3)n2)cc1]

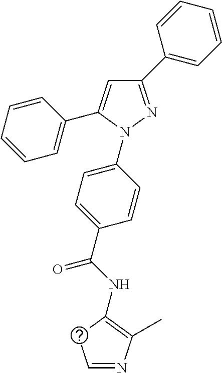 [SMILES: Cc1ncsc1NC(=O)c1ccc(-n2nc(-c3ccccc3)cc2-c2ccccc2)cc1]

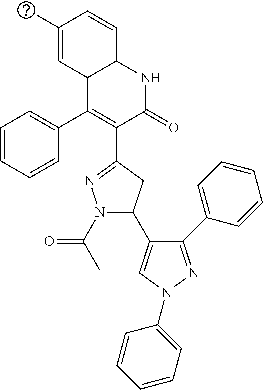 [SMILES: CC(=O)N1N=C(C2=C(c3ccccc3)C3C=C(O)C=CC3NC2=O)CC1c1cn(-c2ccccc2)nc1-c1ccccc1]

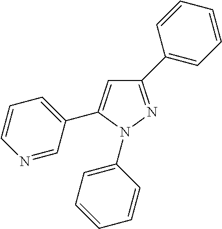 [SMILES: c1ccc(-c2cc(-c3cccnc3)n(-c3ccccc3)n2)cc1]

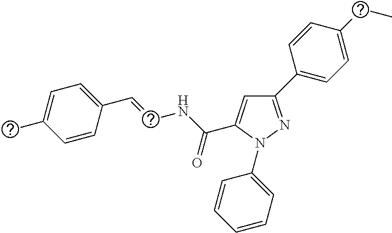 [SMILES: COc1ccc(-c2cc(C(=O)N/N=C/c3ccc(Cl)cc3)n(-c3ccccc3)n2)cc1]

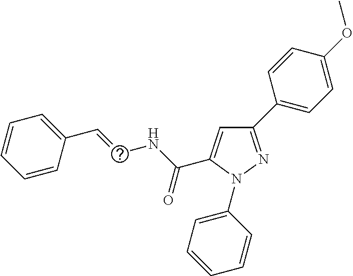 [SMILES: COc1ccc(-c2cc(C(=O)N/N=C/c3ccccc3)n(-c3ccccc3)n2)cc1]

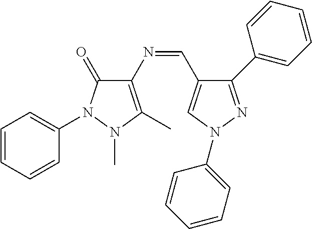 [SMILES: Cc1c(/N=C\c2cn(-c3ccccc3)nc2-c2ccccc2)c(=O)n(-c2ccccc2)n1C]

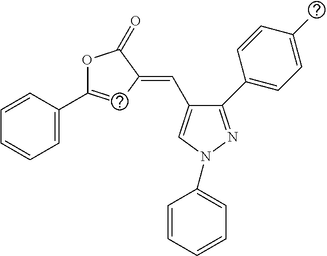

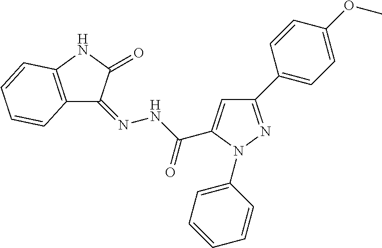 [SMILES: COc1ccc(-c2cc(C(=O)N/N=C3\C(=O)Nc4ccccc43)n(-c3ccccc3)n2)cc1]

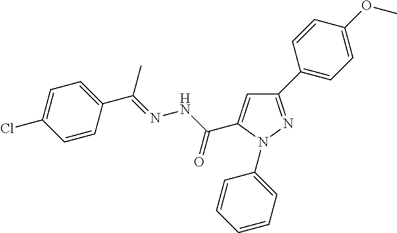 [SMILES: COc1ccc(-c2cc(C(=O)N/N=C(\C)c3ccc(Cl)cc3)n(-c3ccccc3)n2)cc1]

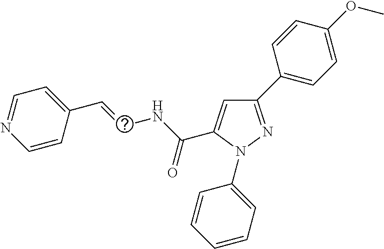 [SMILES: COc1ccc(-c2cc(C(=O)N/N=C/c3ccncc3)n(-c3ccccc3)n2)cc1]

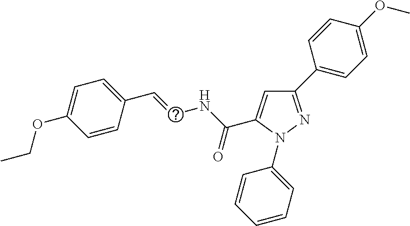 [SMILES: CCOc1ccc(/C=N/NC(=O)c2cc(-c3ccc(OC)cc3)nn2-c2ccccc2)cc1]

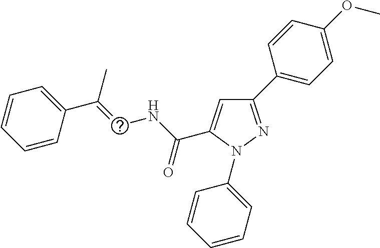 [SMILES: COc1ccc(-c2cc(C(=O)N/N=C(\C)c3ccccc3)n(-c3ccccc3)n2)cc1]

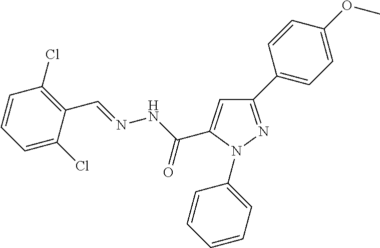 [SMILES: COc1ccc(-c2cc(C(=O)N/N=C/c3c(Cl)cccc3Cl)n(-c3ccccc3)n2)cc1]

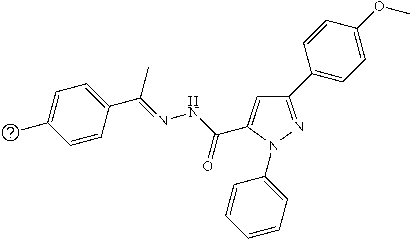 [SMILES: COc1ccc(-c2cc(C(=O)N/N=C(\C)c3ccc(Br)cc3)n(-c3ccccc3)n2)cc1]

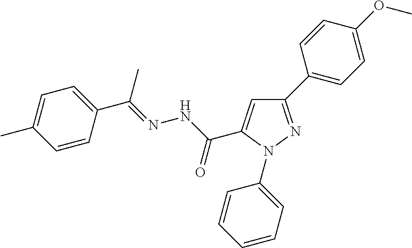

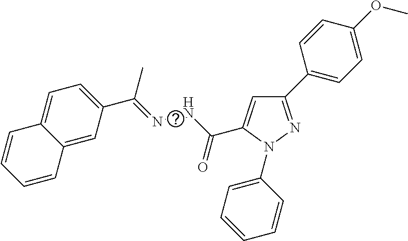 [SMILES: COc1ccc(-c2cc(C(=O)N/N=C(\C)c3ccc4ccccc4c3)n(-c3ccccc3)n2)cc1]

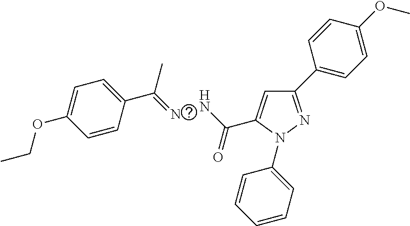 [SMILES: CCOc1ccc(/C(C)=N/NC(=O)c2cc(-c3ccc(OC)cc3)nn2-c2ccccc2)cc1]

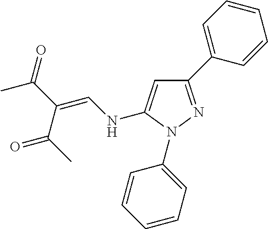 [SMILES: CC(=O)C(=CNc1cc(-c2ccccc2)nn1-c1ccccc1)C(C)=O]

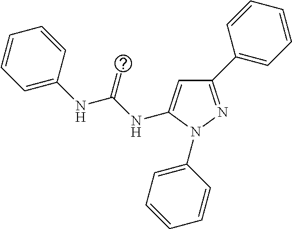 [SMILES: S=C(Nc1ccccc1)Nc1cc(-c2ccccc2)nn1-c1ccccc1]

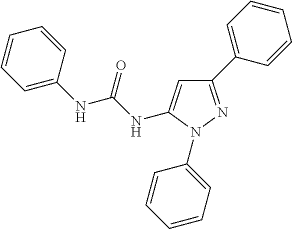 [SMILES: O=C(Nc1ccccc1)Nc1cc(-c2ccccc2)nn1-c1ccccc1]

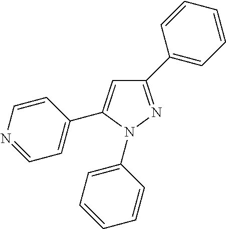 [SMILES: c1ccc(-c2cc(-c3ccncc3)n(-c3ccccc3)n2)cc1]

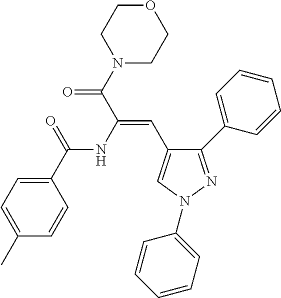 [SMILES: Cc1ccc(C(=O)N/C(=C\c2cn(-c3ccccc3)nc2-c2ccccc2)C(=O)N2CCOCC2)cc1]

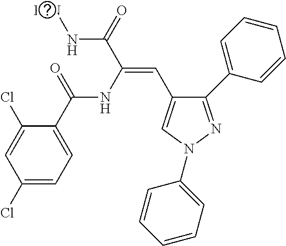 [SMILES: NNC(=O)/C(=C/c1cn(-c2ccccc2)nc1-c1ccccc1)NC(=O)c1ccc(Cl)cc1Cl]

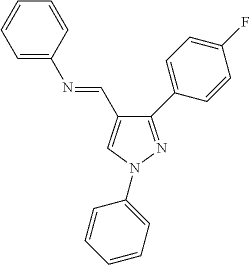 [SMILES: Fc1ccc(-c2nn(-c3ccccc3)cc2/C=N/c2ccccc2)cc1]

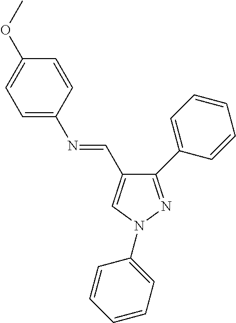 [SMILES: COc1ccc(/N=C/c2cn(-c3ccccc3)nc2-c2ccccc2)cc1]

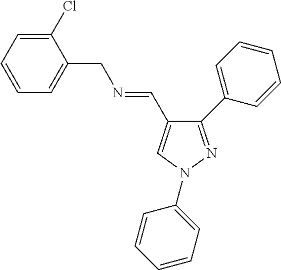 [SMILES: Clc1ccccc1C/N=C/c1cn(-c2ccccc2)nc1-c1ccccc1]

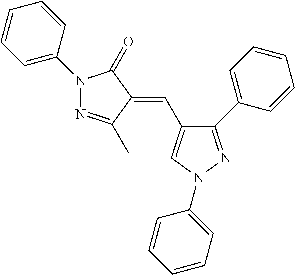 [SMILES: CC1=NN(c2ccccc2)C(=O)/C1=C/c1cn(-c2ccccc2)nc1-c1ccccc1]

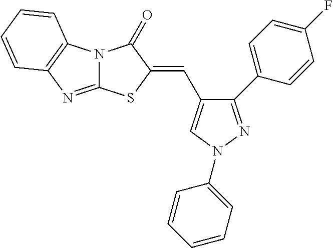 [SMILES: O=c1/c(=C/c2cn(-c3ccccc3)nc2-c2ccc(F)cc2)sc2nc3ccccc3n12]

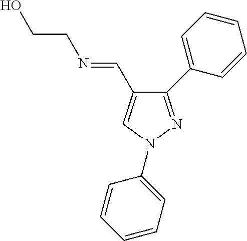 [SMILES: OCC/N=C/c1cn(-c2ccccc2)nc1-c1ccccc1]

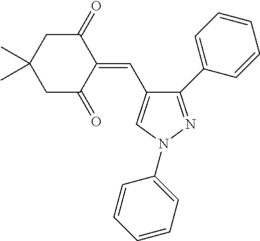 [SMILES: CC1(C)CC(=O)C(=Cc2cn(-c3ccccc3)nc2-c2ccccc2)C(=O)C1]

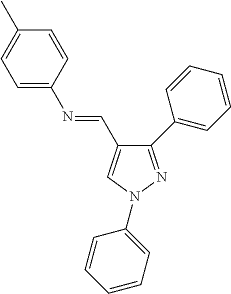 [SMILES: Cc1ccc(/N=C/c2cn(-c3ccccc3)nc2-c2ccccc2)cc1]

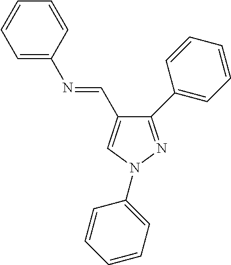 [SMILES: C(=N\c1ccccc1)/c1cn(-c2ccccc2)nc1-c1ccccc1]

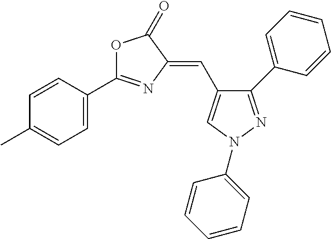 [SMILES: Cc1ccc(C2=N/C(=C\c3cn(-c4ccccc4)nc3-c3ccccc3)C(=O)O2)cc1]

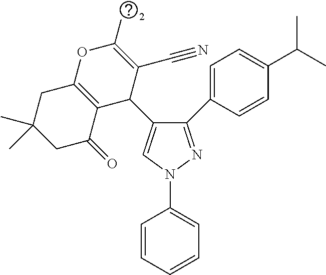 [SMILES: CC(C)c1ccc(-c2nn(-c3ccccc3)cc2C2C(C#N)=C(N)OC3=C2C(=O)CC(C)(C)C3)cc1]

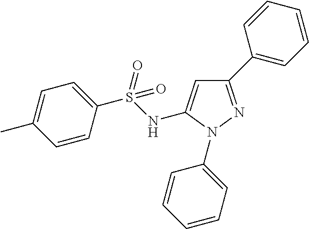 [SMILES: Cc1ccc(S(=O)(=O)Nc2cc(-c3ccccc3)nn2-c2ccccc2)cc1]

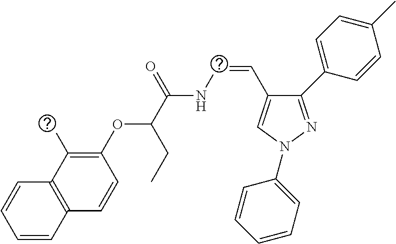 [SMILES: CCC(Oc1ccc2ccccc2c1Br)C(=O)N/N=C\c1cn(-c2ccccc2)nc1-c1ccc(C)cc1]

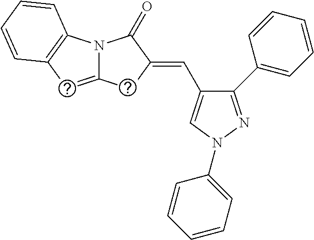 [SMILES: O=c1/c(=C/c2cn(-c3ccccc3)nc2-c2ccccc2)sc2nc3ccccc3n12]

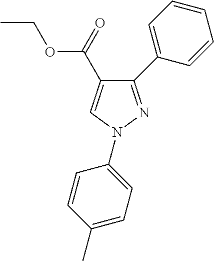 [SMILES: CCOC(=O)c1cn(-c2ccc(C)cc2)nc1-c1ccccc1]

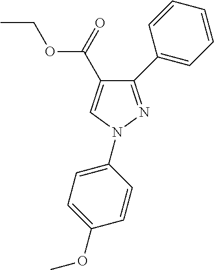 [SMILES: CCOC(=O)c1cn(-c2ccc(OC)cc2)nc1-c1ccccc1]

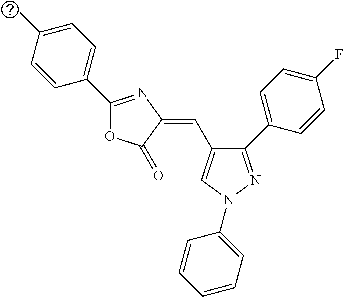 [SMILES: O=C1OC(c2ccc(Br)cc2)=N/C1=C/c1cn(-c2ccccc2)nc1-c1ccc(F)cc1]

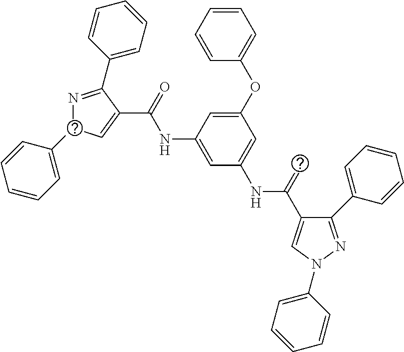 [SMILES: O=C(Nc1cc(NC(=O)c2cn(-c3ccccc3)nc2-c2ccccc2)cc(Oc2ccccc2)c1)c1cn(-c2ccccc2)nc1-c1ccccc1]

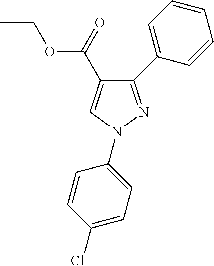 [SMILES: CCOC(=O)c1cn(-c2ccc(Cl)cc2)nc1-c1ccccc1]

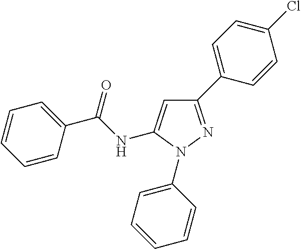 [SMILES: O=C(Nc1cc(-c2ccc(Cl)cc2)nn1-c1ccccc1)c1ccccc1]

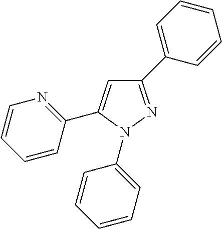 [SMILES: c1ccc(-c2cc(-c3ccccn3)n(-c3ccccc3)n2)cc1]

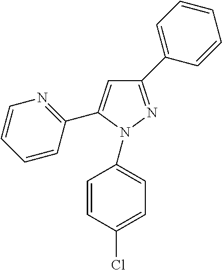 [SMILES: Clc1ccc(-n2nc(-c3ccccc3)cc2-c2ccccn2)cc1]

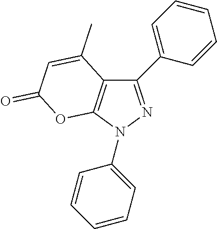 [SMILES: Cc1cc(=O)oc2c1c(-c1ccccc1)nn2-c1ccccc1]

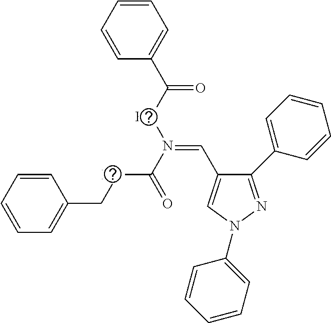

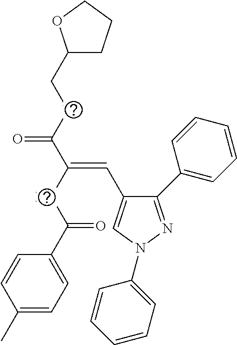 [SMILES: Cc1ccc(C(=O)N/C(=C\c2cn(-c3ccccc3)nc2-c2ccccc2)C(=O)NCC2CCCO2)cc1]

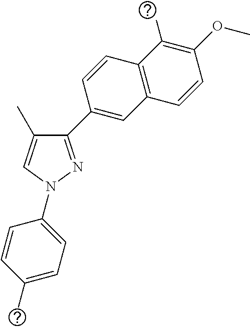 [SMILES: COc1ccc2cc(-c3nn(-c4ccc(F)cc4)cc3C)ccc2c1Br]

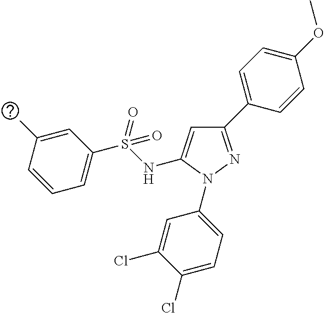 [SMILES: COc1ccc(-c2cc(NS(=O)(=O)c3cccc(F)c3)n(-c3ccc(Cl)c(Cl)c3)n2)cc1]

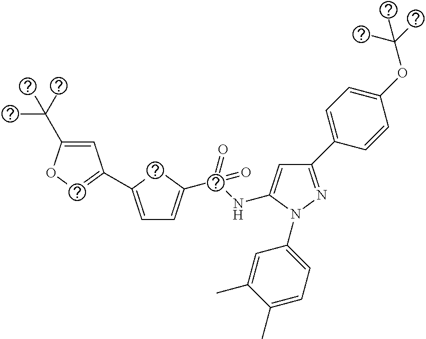 [SMILES: Cc1ccc(-n2nc(-c3ccc(OC(F)(F)F)cc3)cc2NS(=O)(=O)c2ccc(-c3cc(C(F)(F)F)on3)s2)cc1C]

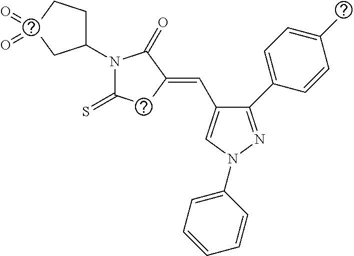 [SMILES: O=C1/C(=C/c2cn(-c3ccccc3)nc2-c2ccc(F)cc2)SC(=S)N1C1CCS(=O)(=O)C1]

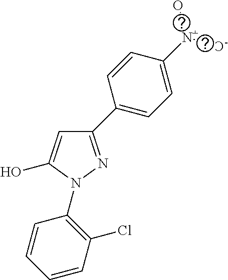 [SMILES: [O-][NH+](O)c1ccc(-c2cc(O)n(-c3ccccc3Cl)n2)cc1]

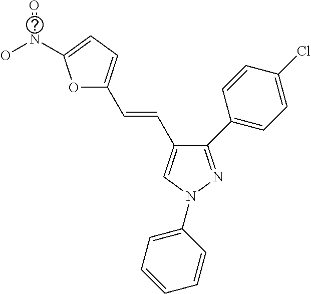

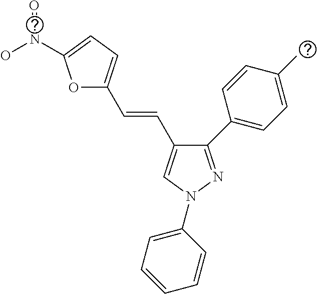

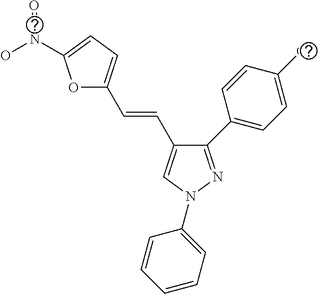

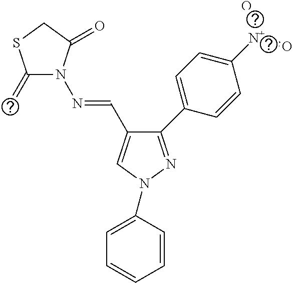

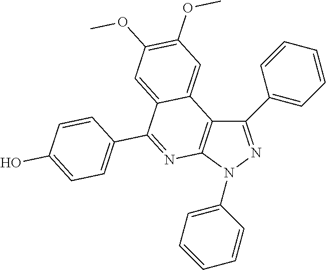 [SMILES: COc1cc2c(-c3ccc(O)cc3)nc3c(c(-c4ccccc4)nn3-c3ccccc3)c2cc1OC]

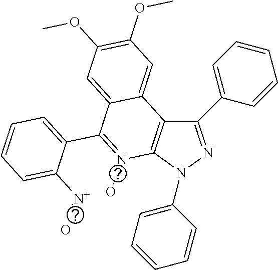

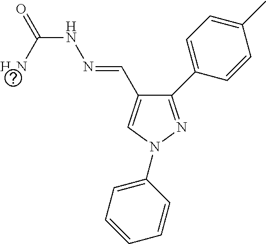 [SMILES: Cc1ccc(-c2nn(-c3ccccc3)cc2/C=N/NC(N)=O)cc1]

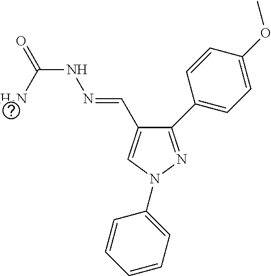 [SMILES: COc1ccc(-c2nn(-c3ccccc3)cc2/C=N/NC(N)=O)cc1]

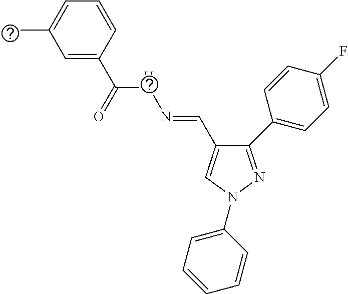 [SMILES: O=C(N/N=C/c1cn(-c2ccccc2)nc1-c1ccc(F)cc1)c1cccc(Br)c1]

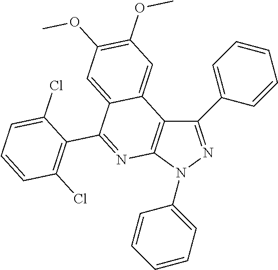 [SMILES: COc1cc2c(-c3c(Cl)cccc3Cl)nc3c(c(-c4ccccc4)nn3-c3ccccc3)c2cc1OC]

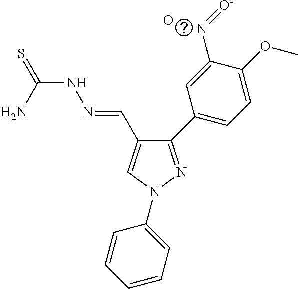

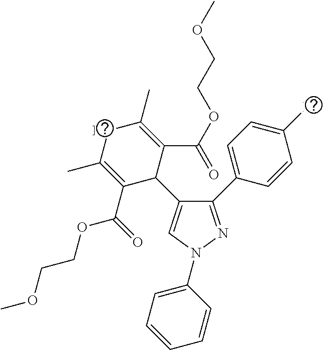 [SMILES: COCCOC(=O)C1=C(C)NC(C)=C(C(=O)OCCOC)C1c1cn(-c2ccccc2)nc1-c1ccc(F)cc1]

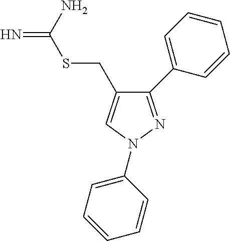 [SMILES: N=C(N)SCc1cn(-c2ccccc2)nc1-c1ccccc1]

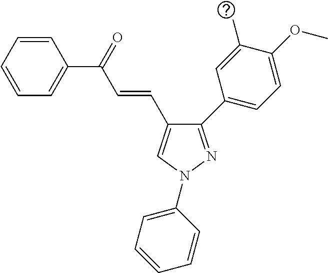 [SMILES: COc1ccc(-c2nn(-c3ccccc3)cc2/C=C/C(=O)c2ccccc2)cc1Br]

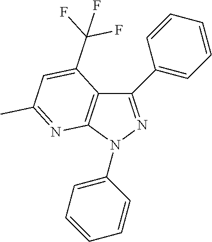 [SMILES: Cc1cc(C(F)(F)F)c2c(-c3ccccc3)nn(-c3ccccc3)c2n1]

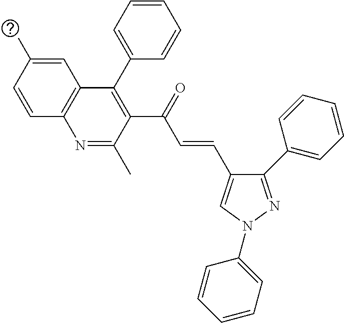 [SMILES: Cc1nc2ccc(Cl)cc2c(-c2ccccc2)c1C(=O)/C=C/c1cn(-c2ccccc2)nc1-c1ccccc1]

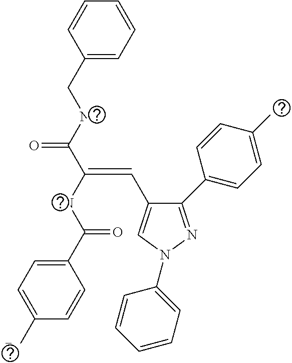 [SMILES: O=C(NCc1ccccc1)/C(=C/c1cn(-c2ccccc2)nc1-c1ccc(F)cc1)NC(=O)c1ccc(Br)cc1]

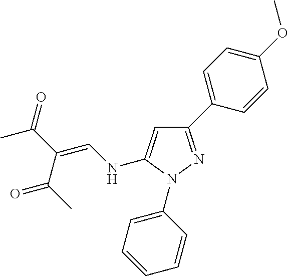 [SMILES: COc1ccc(-c2cc(NC=C(C(C)=O)C(C)=O)n(-c3ccccc3)n2)cc1]

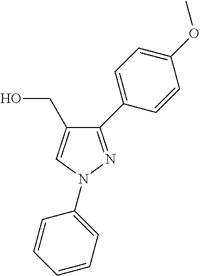 [SMILES: COc1ccc(-c2nn(-c3ccccc3)cc2CO)cc1]

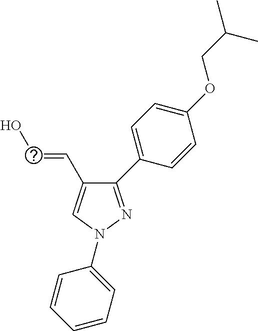 [SMILES: CC(C)COc1ccc(-c2nn(-c3ccccc3)cc2/C=N/O)cc1]

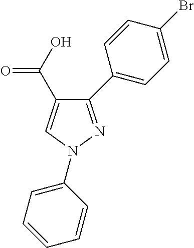 [SMILES: O=C(O)c1cn(-c2ccccc2)nc1-c1ccc(Br)cc1]

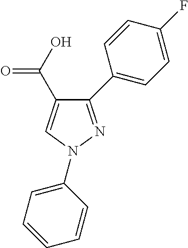 [SMILES: O=C(O)c1cn(-c2ccccc2)nc1-c1ccc(F)cc1]

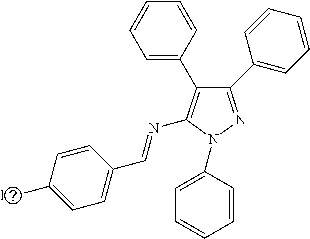 [SMILES: Oc1ccc(/C=N/c2c(-c3ccccc3)c(-c3ccccc3)nn2-c2ccccc2)cc1]

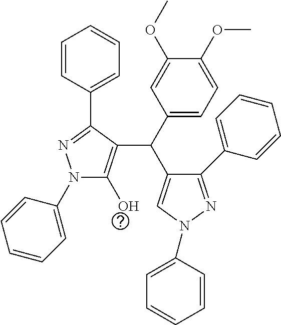 [SMILES: COc1ccc(C(c2cn(-c3ccccc3)nc2-c2ccccc2)c2c(-c3ccccc3)nn(-c3ccccc3)c2O)cc1OC]

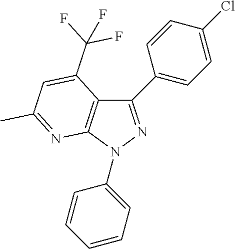 [SMILES: Cc1cc(C(F)(F)F)c2c(-c3ccc(Cl)cc3)nn(-c3ccccc3)c2n1]

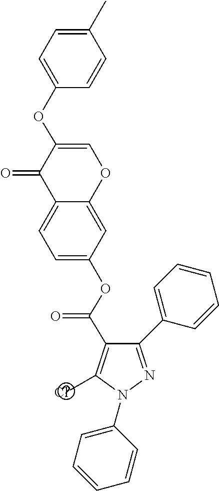 [SMILES: Cc1ccc(Oc2coc3cc(OC(=O)c4c(-c5ccccc5)nn(-c5ccccc5)c4Cl)ccc3c2=O)cc1]

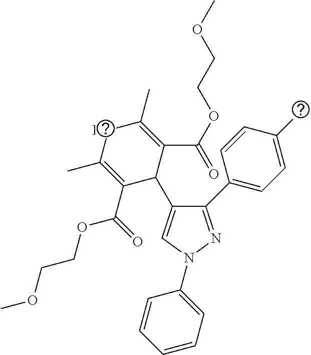 [SMILES: COCCOC(=O)C1=C(C)NC(C)=C(C(=O)OCCOC)C1c1cn(-c2ccccc2)nc1-c1ccc(Br)cc1]

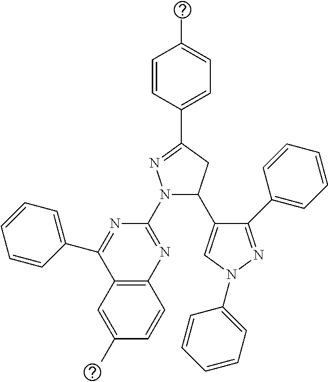 [SMILES: Clc1ccc2nc(N3N=C(c4ccc(Br)cc4)CC3c3cn(-c4ccccc4)nc3-c3ccccc3)nc(-c3ccccc3)c2c1]

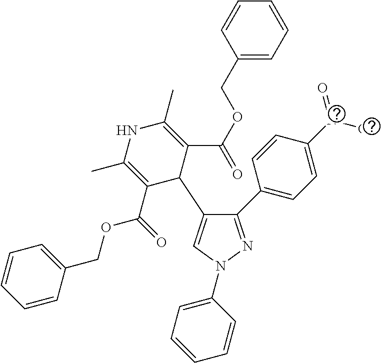 [SMILES: CC1=C(C(=O)OCc2ccccc2)C(c2cn(-c3ccccc3)nc2-c2ccc([N+](=O)[O-])cc2)C(C(=O)OCc2ccccc2)=C(C)N1]

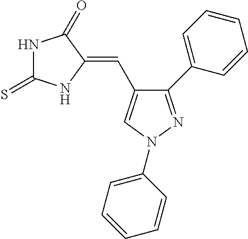 [SMILES: O=C1NC(=S)N/C1=C\c1cn(-c2ccccc2)nc1-c1ccccc1]

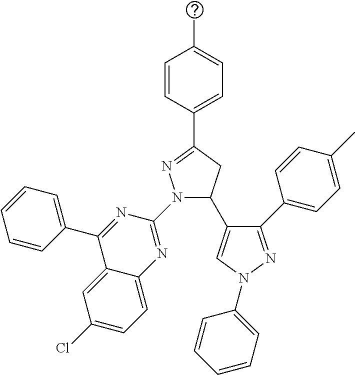 [SMILES: Cc1ccc(-c2nn(-c3ccccc3)cc2C2CC(c3ccc(Br)cc3)=NN2c2nc(-c3ccccc3)c3cc(Cl)ccc3n2)cc1]

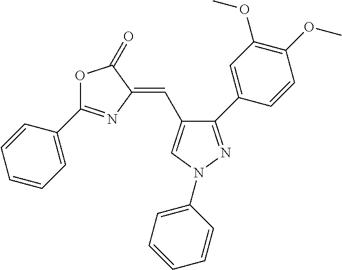 [SMILES: COc1ccc(-c2nn(-c3ccccc3)cc2/C=C2\N=C(c3ccccc3)OC2=O)cc1OC]

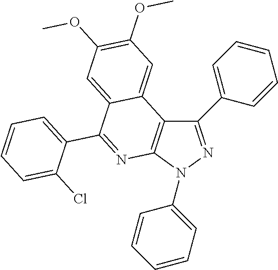 [SMILES: COc1cc2c(-c3ccccc3Cl)nc3c(c(-c4ccccc4)nn3-c3ccccc3)c2cc1OC]

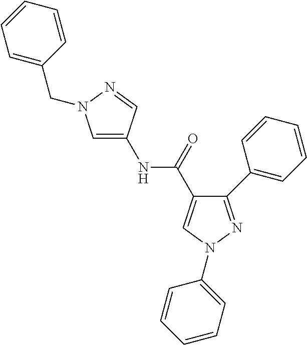 [SMILES: O=C(Nc1cnn(Cc2ccccc2)c1)c1cn(-c2ccccc2)nc1-c1ccccc1]

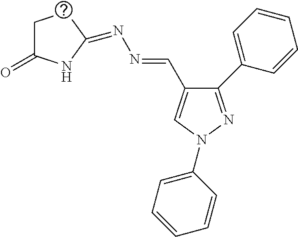 [SMILES: O=C1CS/C(=N/N=C/c2cn(-c3ccccc3)nc2-c2ccccc2)N1]

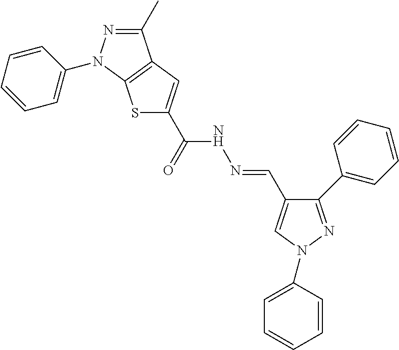 [SMILES: Cc1nn(-c2ccccc2)c2sc(C(=O)N/N=C/c3cn(-c4ccccc4)nc3-c3ccccc3)cc12]

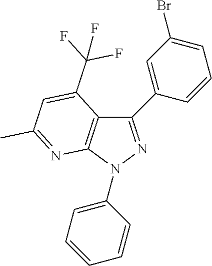 [SMILES: Cc1cc(C(F)(F)F)c2c(-c3cccc(Br)c3)nn(-c3ccccc3)c2n1]

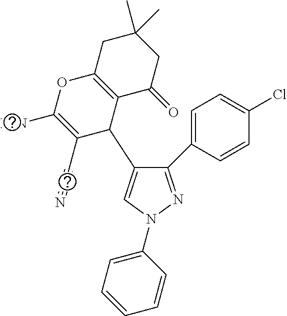 [SMILES: CC1(C)CC(=O)C2=C(C1)OC(N)=C(C#N)C2c1cn(-c2ccccc2)nc1-c1ccc(Cl)cc1]

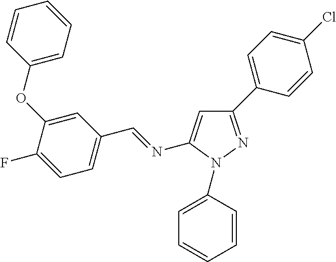 [SMILES: Fc1ccc(/C=N/c2cc(-c3ccc(Cl)cc3)nn2-c2ccccc2)cc1Oc1ccccc1]

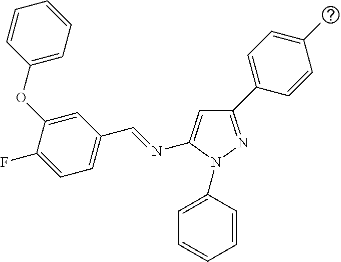 [SMILES: Fc1ccc(/C=N/c2cc(-c3ccc(Br)cc3)nn2-c2ccccc2)cc1Oc1ccccc1]

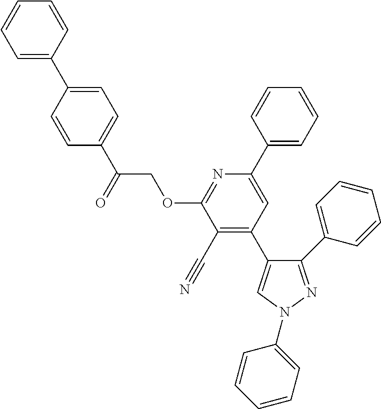 [SMILES: N#Cc1c(-c2cn(-c3ccccc3)nc2-c2ccccc2)cc(-c2ccccc2)nc1OCC(=O)c1ccc(-c2ccccc2)cc1]

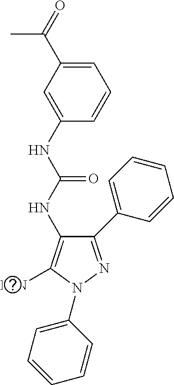 [SMILES: CC(=O)c1cccc(NC(=O)Nc2c(-c3ccccc3)nn(-c3ccccc3)c2N)c1]

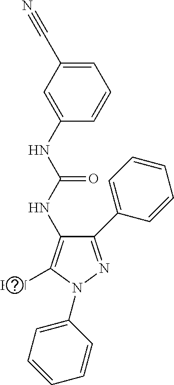 [SMILES: N#Cc1cccc(NC(=O)Nc2c(-c3ccccc3)nn(-c3ccccc3)c2N)c1]

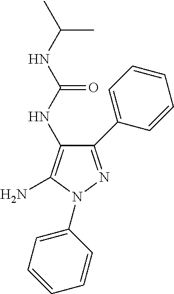 [SMILES: CC(C)NC(=O)Nc1c(-c2ccccc2)nn(-c2ccccc2)c1N]

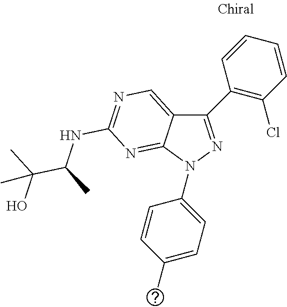 [SMILES: C[C@H](Nc1ncc2c(-c3ccccc3Cl)nn(-c3ccc(F)cc3)c2n1)C(C)(C)O]

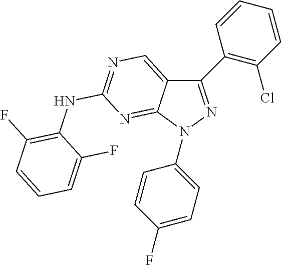 [SMILES: Fc1ccc(-n2nc(-c3ccccc3Cl)c3cnc(Nc4c(F)cccc4F)nc32)cc1]

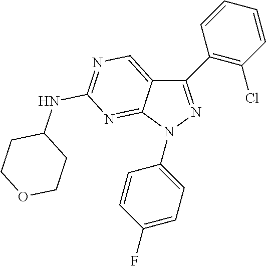 [SMILES: Fc1ccc(-n2nc(-c3ccccc3Cl)c3cnc(NC4CCOCC4)nc32)cc1]

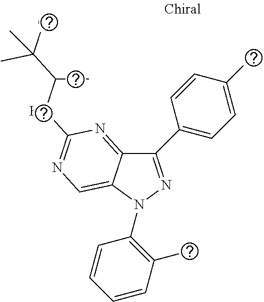 [SMILES: CC(Nc1ncc2c(n1)c(-c1ccc(F)cc1)nn2-c1ccccc1Cl)C(C)(C)O]

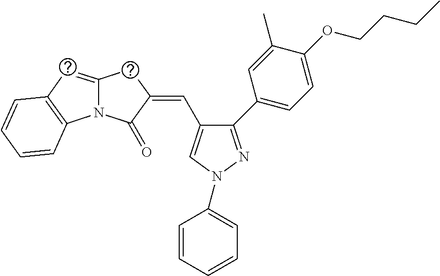 [SMILES: CCCCOc1ccc(-c2nn(-c3ccccc3)cc2/C=c2/sc3nc4ccccc4n3c2=O)cc1C]

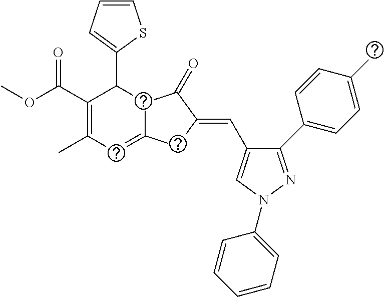 [SMILES: COC(=O)C1=C(C)N=c2s/c(=C\c3cn(-c4ccccc4)nc3-c3ccc(Cl)cc3)c(=O)n2C1c1cccs1]

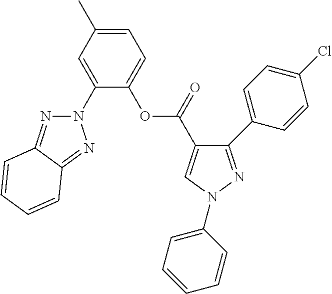 [SMILES: Cc1ccc(OC(=O)c2cn(-c3ccccc3)nc2-c2ccc(Cl)cc2)c(-n2nc3ccccc3n2)c1]

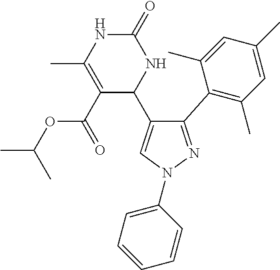 [SMILES: CC1=C(C(=O)OC(C)C)C(c2cn(-c3ccccc3)nc2-c2c(C)cc(C)cc2C)NC(=O)N1]

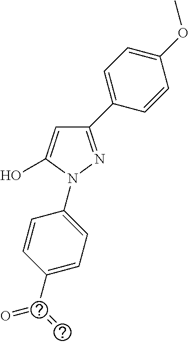

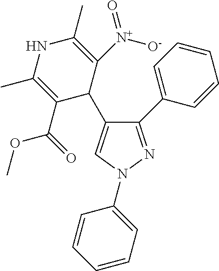 [SMILES: COC(=O)C1=C(C)NC(C)=C([N+](=O)[O-])C1c1cn(-c2ccccc2)nc1-c1ccccc1]

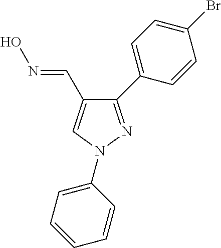 [SMILES: O/N=C/c1cn(-c2ccccc2)nc1-c1ccc(Br)cc1]

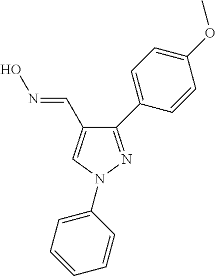 [SMILES: COc1ccc(-c2nn(-c3ccccc3)cc2/C=N/O)cc1]

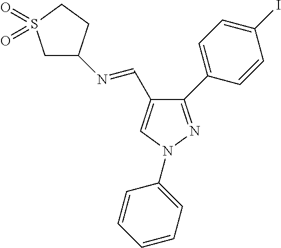 [SMILES: O=S1(=O)CCC(/N=C/c2cn(-c3ccccc3)nc2-c2ccc(I)cc2)C1]

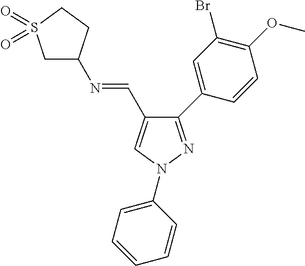 [SMILES: COc1ccc(-c2nn(-c3ccccc3)cc2/C=N/C2CCS(=O)(=O)C2)cc1Br]

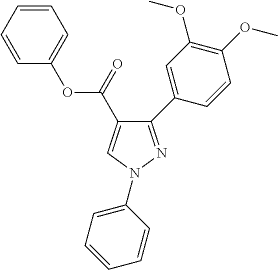 [SMILES: COc1ccc(-c2nn(-c3ccccc3)cc2C(=O)Oc2ccccc2)cc1OC]

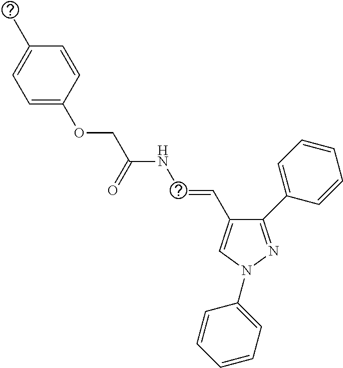 [SMILES: Cc1ccc(OCC(=O)N/N=C/c2cn(-c3ccccc3)nc2-c2ccccc2)cc1]

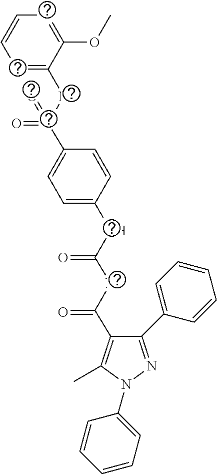 [SMILES: COc1nccnc1NS(=O)(=O)c1ccc(NC(=O)NC(=O)c2c(-c3ccccc3)nn(-c3ccccc3)c2C)cc1]

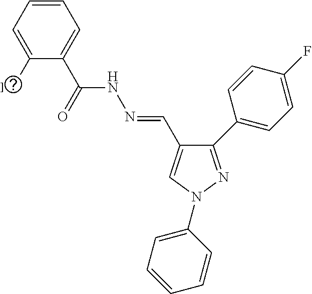 [SMILES: O=C(N/N=C/c1cn(-c2ccccc2)nc1-c1ccc(F)cc1)c1ccccc1Br]

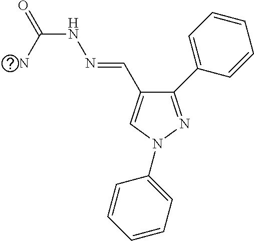 [SMILES: NC(=O)N/N=C/c1cn(-c2ccccc2)nc1-c1ccccc1]

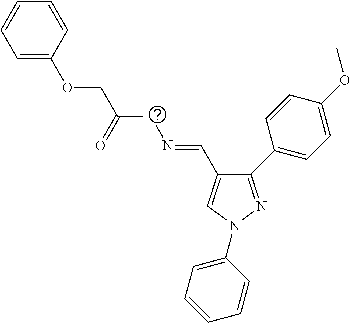 [SMILES: COc1ccc(-c2nn(-c3ccccc3)cc2/C=N/NC(=O)COc2ccccc2)cc1]

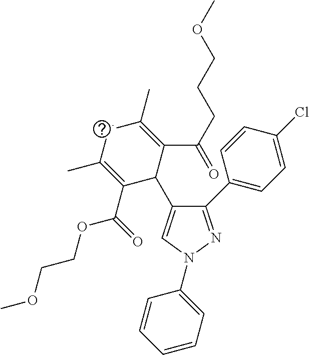 [SMILES: COCCCC(=O)C1=C(C)NC(C)=C(C(=O)OCCOC)C1c1cn(-c2ccccc2)nc1-c1ccc(Cl)cc1]

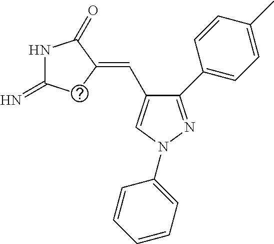 [SMILES: Cc1ccc(-c2nn(-c3ccccc3)cc2/C=C2\SC(=N)NC2=O)cc1]

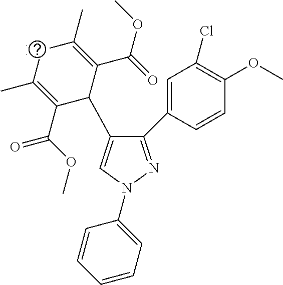

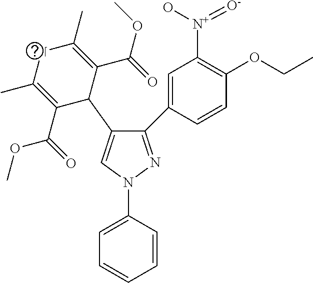

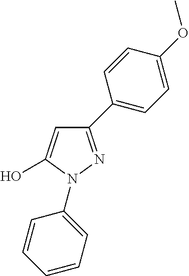 [SMILES: COc1ccc(-c2cc(O)n(-c3ccccc3)n2)cc1]

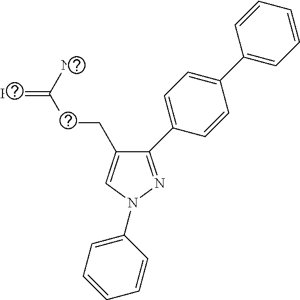 [SMILES: N=C(N)SCc1cn(-c2ccccc2)nc1-c1ccc(-c2ccccc2)cc1]

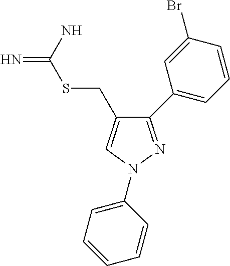 [SMILES: N=C(N)SCc1cn(-c2ccccc2)nc1-c1cccc(Br)c1]

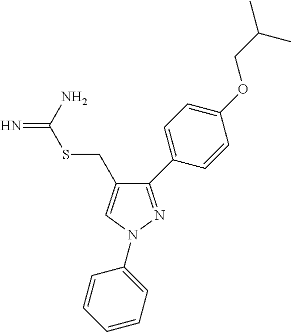 [SMILES: CC(C)COc1ccc(-c2nn(-c3ccccc3)cc2CSC(=N)N)cc1]

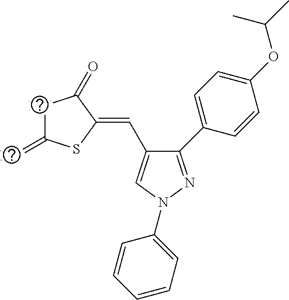 [SMILES: CC(C)Oc1ccc(-c2nn(-c3ccccc3)cc2/C=C2\SC(=N)NC2=O)cc1]

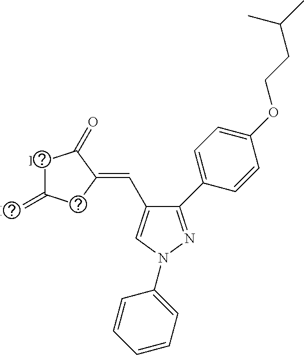 [SMILES: CC(C)CCOc1ccc(-c2nn(-c3ccccc3)cc2/C=C2\SC(=N)NC2=O)cc1]

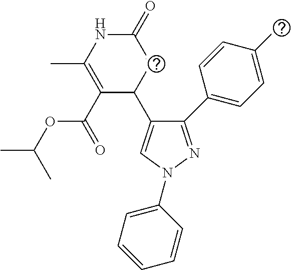 [SMILES: CC1=C(C(=O)OC(C)C)C(c2cn(-c3ccccc3)nc2-c2ccc(Br)cc2)NC(=O)N1]

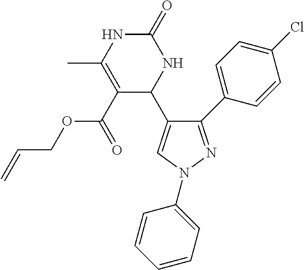 [SMILES: C=CCOC(=O)C1=C(C)NC(=O)NC1c1cn(-c2ccccc2)nc1-c1ccc(Cl)cc1]

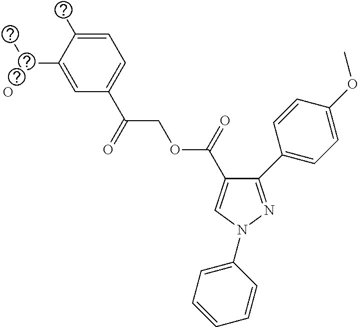 [SMILES: COc1ccc(-c2nn(-c3ccccc3)cc2C(=O)OCC(=O)c2ccc(C)c([NH+]([O-])O)c2)cc1]

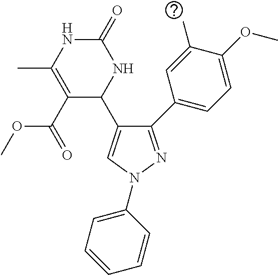

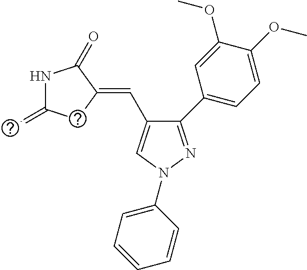 [SMILES: COc1ccc(-c2nn(-c3ccccc3)cc2/C=C2\SC(=N)NC2=O)cc1OC]

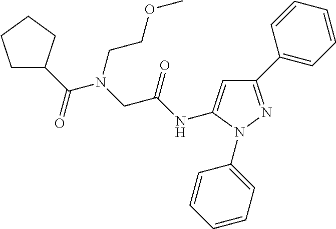 [SMILES: COCCN(CC(=O)Nc1cc(-c2ccccc2)nn1-c1ccccc1)C(=O)C1CCCC1]

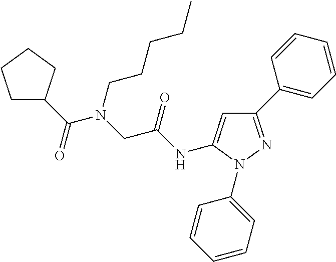 [SMILES: CCCCCN(CC(=O)Nc1cc(-c2ccccc2)nn1-c1ccccc1)C(=O)C1CCCC1]

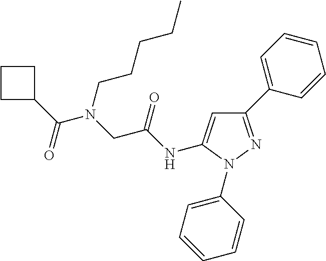 [SMILES: CCCCCN(CC(=O)Nc1cc(-c2ccccc2)nn1-c1ccccc1)C(=O)C1CCC1]

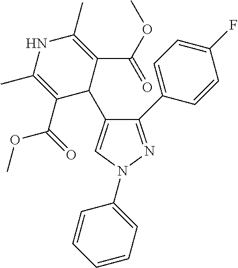 [SMILES: COC(=O)C1=C(C)NC(C)=C(C(=O)OC)C1c1cn(-c2ccccc2)nc1-c1ccc(F)cc1]

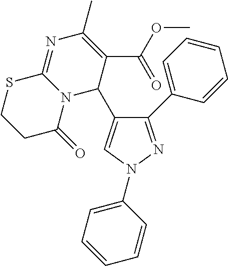 [SMILES: COC(=O)C1=C(C)N=C2SCCC(=O)N2C1c1cn(-c2ccccc2)nc1-c1ccccc1]

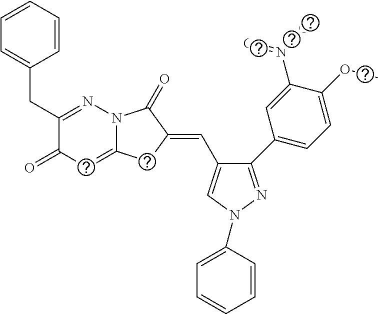

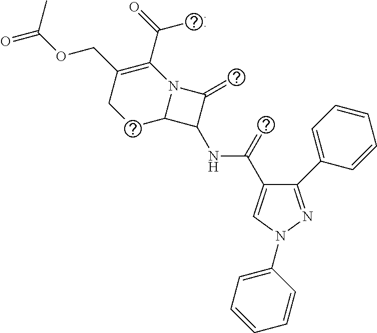 [SMILES: CC(=O)OCC1=C(C(=O)O)N2C(=O)C(NC(=O)c3cn(-c4ccccc4)nc3-c3ccccc3)C2SC1]

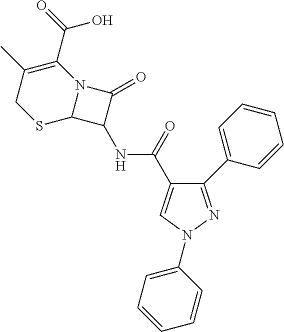 [SMILES: CC1=C(C(=O)O)N2C(=O)C(NC(=O)c3cn(-c4ccccc4)nc3-c3ccccc3)C2SC1]

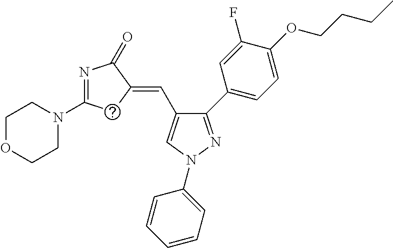 [SMILES: CCCCOc1ccc(-c2nn(-c3ccccc3)cc2/C=C2\SC(N3CCOCC3)=NC2=O)cc1F]

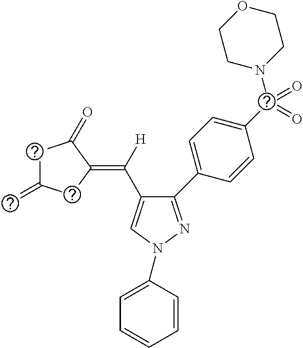 [SMILES: O=C1NC(=S)S/C1=C\c1cn(-c2ccccc2)nc1-c1ccc(S(=O)(=O)N2CCOCC2)cc1]

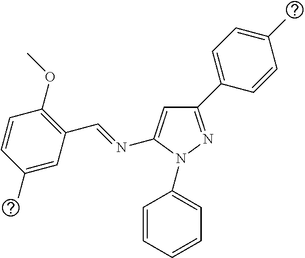 [SMILES: COc1ccc(Br)cc1/C=N/c1cc(-c2ccc(Cl)cc2)nn1-c1ccccc1]

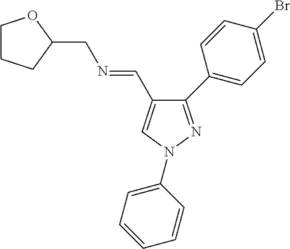 [SMILES: Brc1ccc(-c2nn(-c3ccccc3)cc2/C=N/CC2CCCO2)cc1]

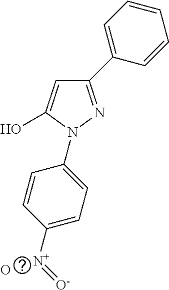

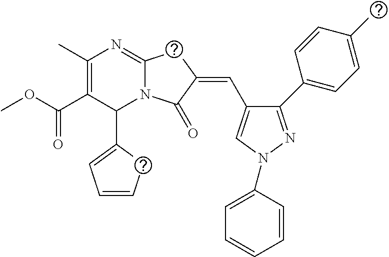 [SMILES: COC(=O)C1=C(C)N=c2s/c(=C/c3cn(-c4ccccc4)nc3-c3ccc(Cl)cc3)c(=O)n2C1c1ccco1]

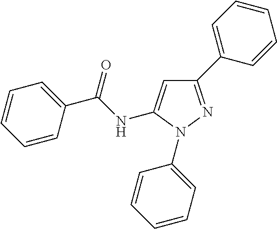 [SMILES: O=C(Nc1cc(-c2ccccc2)nn1-c1ccccc1)c1ccccc1]

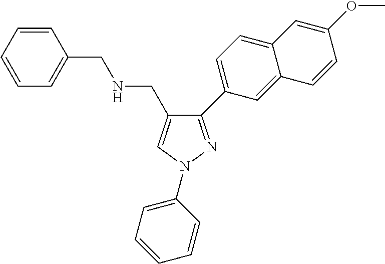 [SMILES: COc1ccc2cc(-c3nn(-c4ccccc4)cc3CNCc3ccccc3)ccc2c1]

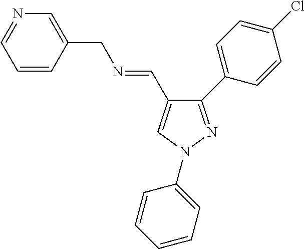 [SMILES: Clc1ccc(-c2nn(-c3ccccc3)cc2/C=N/Cc2cccnc2)cc1]

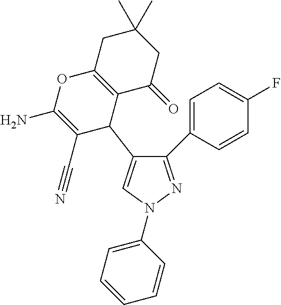 [SMILES: CC1(C)CC(=O)C2=C(C1)OC(N)=C(C#N)C2c1cn(-c2ccccc2)nc1-c1ccc(F)cc1]

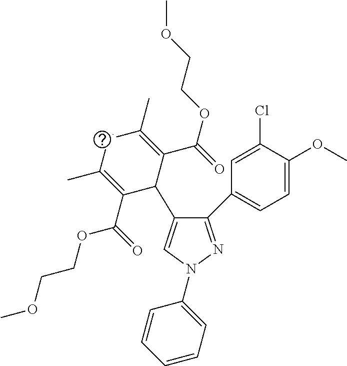

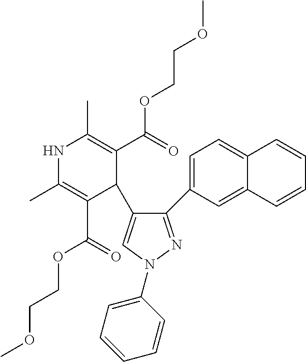 [SMILES: COCCOC(=O)C1=C(C)NC(C)=C(C(=O)OCCOC)C1c1cn(-c2ccccc2)nc1-c1ccc2ccccc2c1]

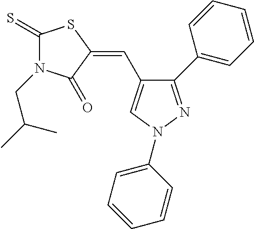 [SMILES: CC(C)CN1C(=O)/C(=C\c2cn(-c3ccccc3)nc2-c2ccccc2)SC1=S]

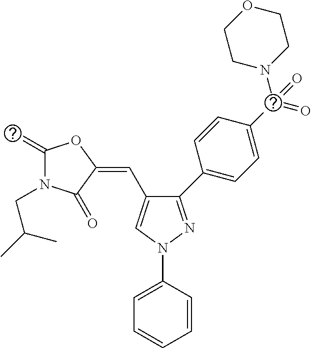 [SMILES: CC(C)CN1C(=O)/C(=C\c2cn(-c3ccccc3)nc2-c2ccc(S(=O)(=O)N3CCOCC3)cc2)OC1=S]

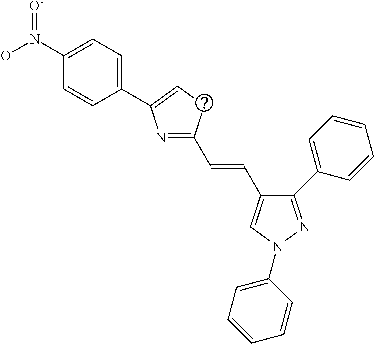

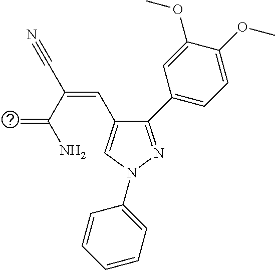 [SMILES: COc1ccc(-c2nn(-c3ccccc3)cc2/C=C(/C#N)C(N)=S)cc1OC]

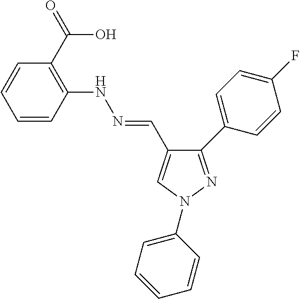 [SMILES: O=C(O)c1ccccc1N/N=C/c1cn(-c2ccccc2)nc1-c1ccc(F)cc1]

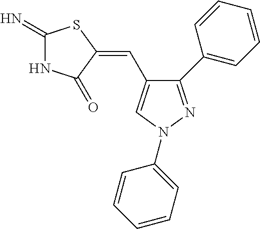 [SMILES: N=C1NC(=O)/C(=C\c2cn(-c3ccccc3)nc2-c2ccccc2)S1]

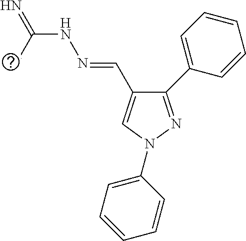 [SMILES: N=C(N)N/N=C/c1cn(-c2ccccc2)nc1-c1ccccc1]

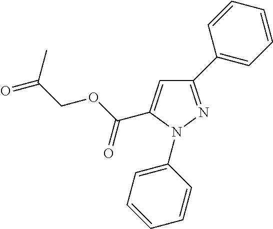 [SMILES: CC(=O)COC(=O)c1cc(-c2ccccc2)nn1-c1ccccc1]

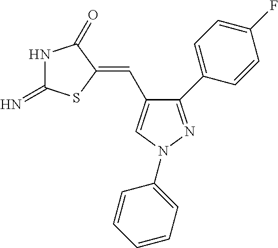 [SMILES: N=C1NC(=O)/C(=C/c2cn(-c3ccccc3)nc2-c2ccc(F)cc2)S1]

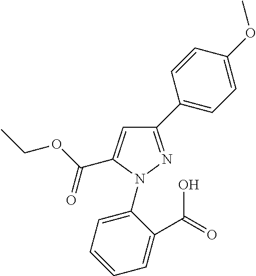 [SMILES: CCOC(=O)c1cc(-c2ccc(OC)cc2)nn1-c1ccccc1C(=O)O]

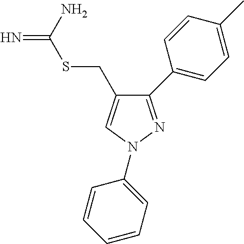 [SMILES: Cc1ccc(-c2nn(-c3ccccc3)cc2CSC(=N)N)cc1]

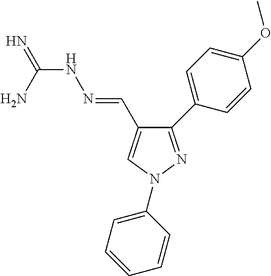 [SMILES: COc1ccc(-c2nn(-c3ccccc3)cc2/C=N/NC(=N)N)cc1]

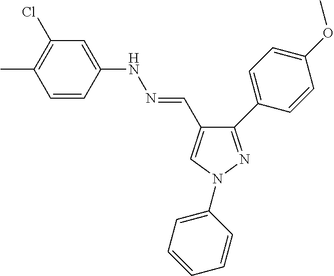 [SMILES: COc1ccc(-c2nn(-c3ccccc3)cc2/C=N/Nc2ccc(C)c(Cl)c2)cc1]

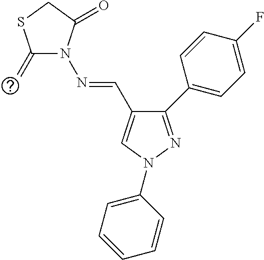 [SMILES: O=C1CSC(=S)N1/N=C/c1cn(-c2ccccc2)nc1-c1ccc(F)cc1]

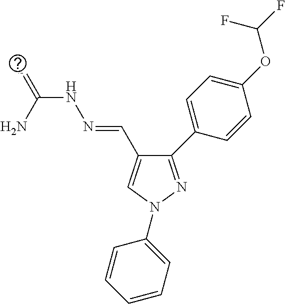 [SMILES: NC(=S)N/N=C/c1cn(-c2ccccc2)nc1-c1ccc(OC(F)F)cc1]

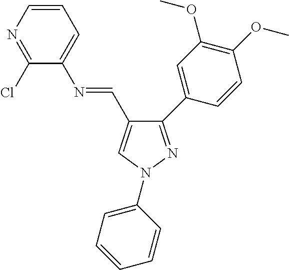 [SMILES: COc1ccc(-c2nn(-c3ccccc3)cc2/C=N/c2cccnc2Cl)cc1OC]

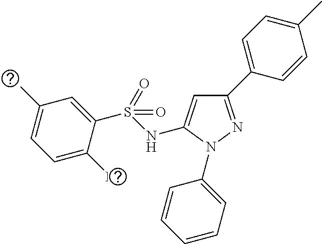 [SMILES: Cc1ccc(-c2cc(NS(=O)(=O)c3cc(Br)ccc3Br)n(-c3ccccc3)n2)cc1]

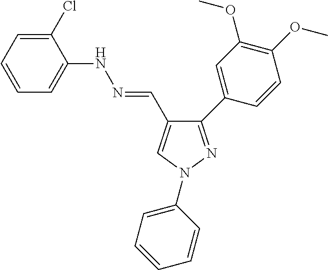 [SMILES: COc1ccc(-c2nn(-c3ccccc3)cc2/C=N/Nc2ccccc2Cl)cc1OC]

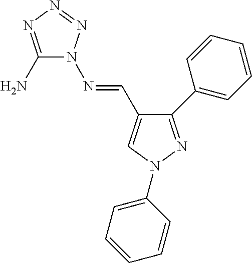 [SMILES: Nc1nnnn1/N=C/c1cn(-c2ccccc2)nc1-c1ccccc1]

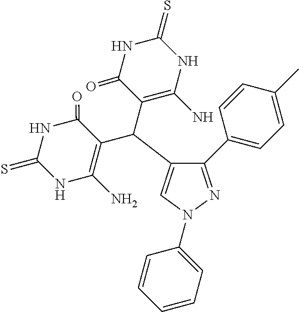 [SMILES: Cc1ccc(-c2nn(-c3ccccc3)cc2C(c2c(N)[nH]c(=S)[nH]c2=O)c2c(N)[nH]c(=S)[nH]c2=O)cc1]

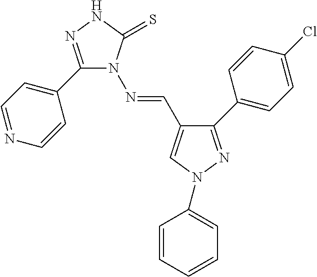 [SMILES: S=c1[nH]nc(-c2ccncc2)n1/N=C/c1cn(-c2ccccc2)nc1-c1ccc(Cl)cc1]

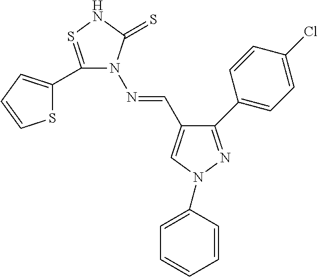

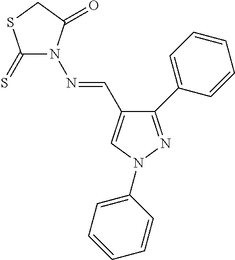 [SMILES: O=C1CSC(=S)N1/N=C/c1cn(-c2ccccc2)nc1-c1ccccc1]

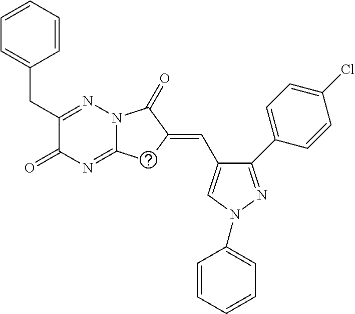 [SMILES: O=c1nc2s/c(=C\c3cn(-c4ccccc4)nc3-c3ccc(Cl)cc3)c(=O)n2nc1Cc1ccccc1]

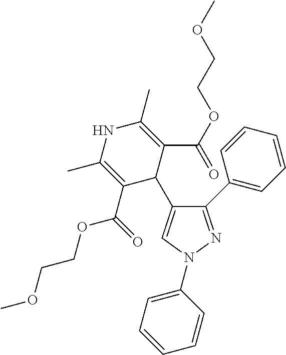 [SMILES: COCCOC(=O)C1=C(C)NC(C)=C(C(=O)OCCOC)C1c1cn(-c2ccccc2)nc1-c1ccccc1]

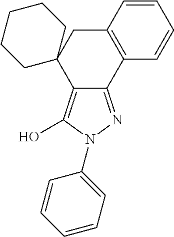 [SMILES: Oc1c2c(nn1-c1ccccc1)-c1ccccc1CC21CCCCC1]

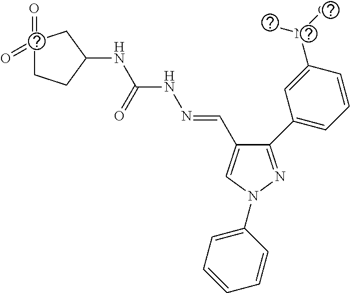 [SMILES: O=C(N/N=C/c1cn(-c2ccccc2)nc1-c1cccc([NH+]([O-])O)c1)NC1CCS(=O)(=O)C1]

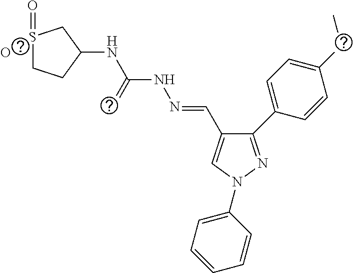 [SMILES: COc1ccc(-c2nn(-c3ccccc3)cc2/C=N/NC(=O)NC2CCS(=O)(=O)C2)cc1]

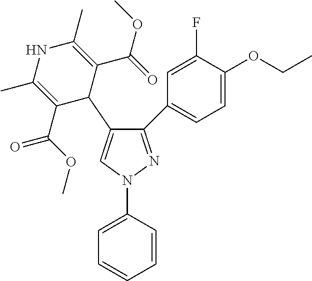 [SMILES: CCOc1ccc(-c2nn(-c3ccccc3)cc2C2C(C(=O)OC)=C(C)NC(C)=C2C(=O)OC)cc1F]

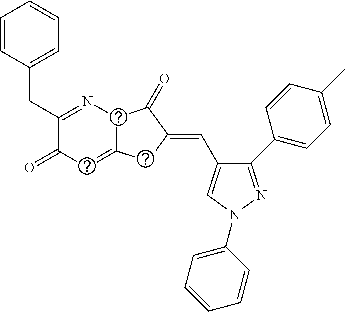 [SMILES: Cc1ccc(-c2nn(-c3ccccc3)cc2/C=c2\sc3nc(=O)c(Cc4ccccc4)nn3c2=O)cc1]

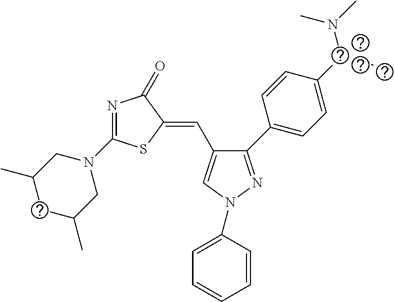

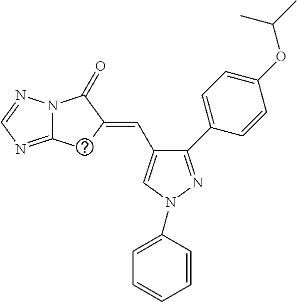 [SMILES: CC(C)Oc1ccc(-c2nn(-c3ccccc3)cc2/C=c2\sc3ncnn3c2=O)cc1]

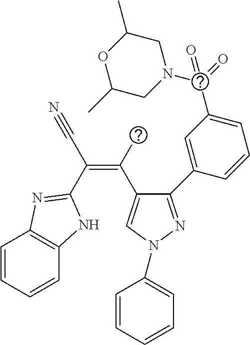 [SMILES: CC1CN(S(=O)(=O)c2cccc(-c3nn(-c4ccccc4)cc3/C=C(/C#N)c3nc4ccccc4[nH]3)c2)CC(C)O1]

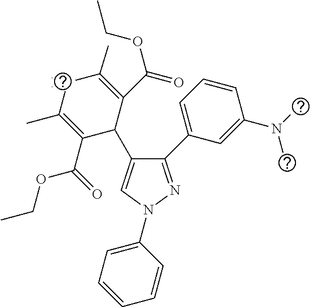 [SMILES: CCOC(=O)C1=C(C)NC(C)=C(C(=O)OCC)C1c1cn(-c2ccccc2)nc1-c1cccc(N([O-])O)c1]

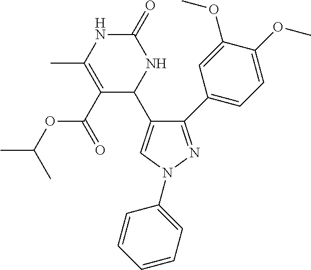 [SMILES: COc1ccc(-c2nn(-c3ccccc3)cc2C2NC(=O)NC(C)=C2C(=O)OC(C)C)cc1OC]

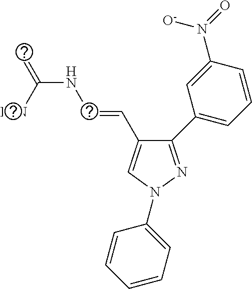

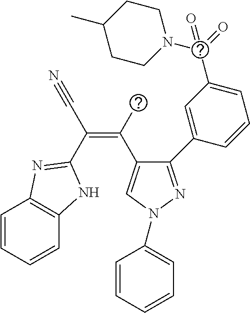 [SMILES: CC1CCN(S(=O)(=O)c2cccc(-c3nn(-c4ccccc4)cc3/C=C(/C#N)c3nc4ccccc4[nH]3)c2)CC1]

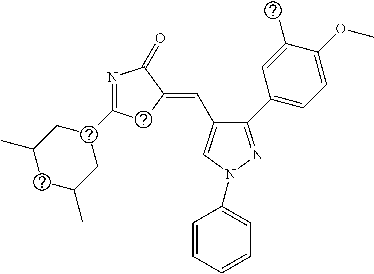

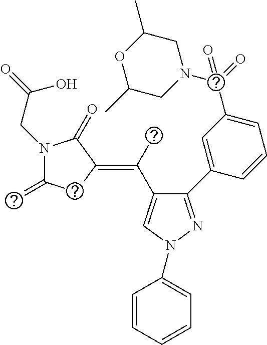 [SMILES: CC1CN(S(=O)(=O)c2cccc(-c3nn(-c4ccccc4)cc3/C=C3\SC(=S)N(CC(=O)O)C3=O)c2)CC(C)O1]

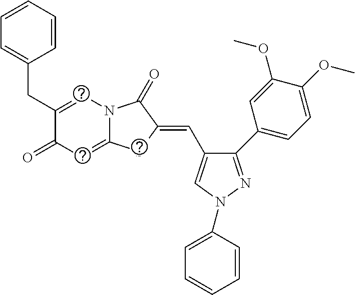 [SMILES: COc1ccc(-c2nn(-c3ccccc3)cc2/C=c2\sc3nc(=O)c(Cc4ccccc4)nn3c2=O)cc1OC]

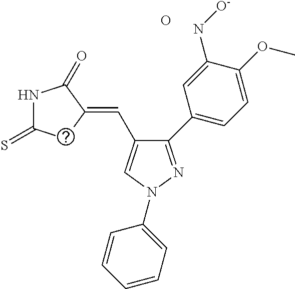 [SMILES: COc1ccc(-c2nn(-c3ccccc3)cc2/C=C2\SC(=S)NC2=O)cc1N[O-] ; O]

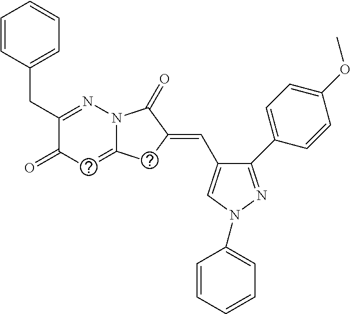 [SMILES: COc1ccc(-c2nn(-c3ccccc3)cc2/C=c2\sc3nc(=O)c(Cc4ccccc4)nn3c2=O)cc1]

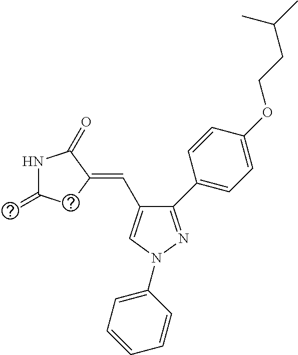 [SMILES: CC(C)CCOc1ccc(-c2nn(-c3ccccc3)cc2/C=C2\SC(=S)NC2=O)cc1]

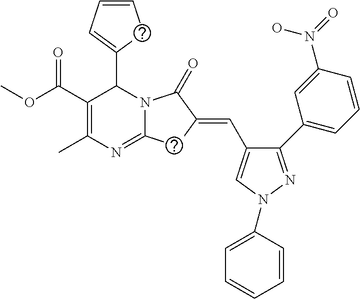

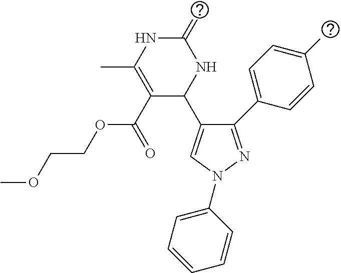 [SMILES: COCCOC(=O)C1=C(C)NC(=O)NC1c1cn(-c2ccccc2)nc1-c1ccc(S)cc1]

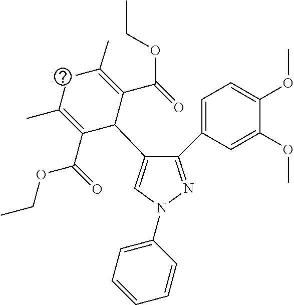 [SMILES: CCOC(=O)C1=C(C)NC(C)=C(C(=O)OCC)C1c1cn(-c2ccccc2)nc1-c1ccc(OC)c(OC)c1]

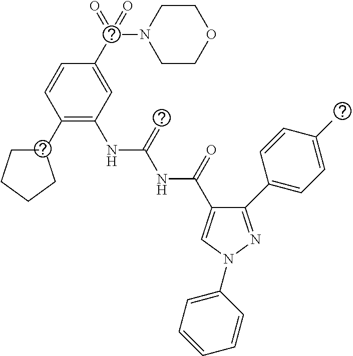

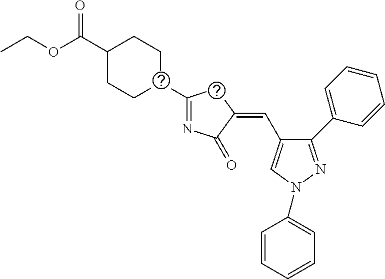 [SMILES: CCOC(=O)C1CCN(C2=NC(=O)/C(=C\c3cn(-c4ccccc4)nc3-c3ccccc3)S2)CC1]

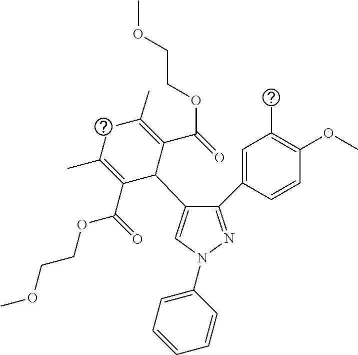

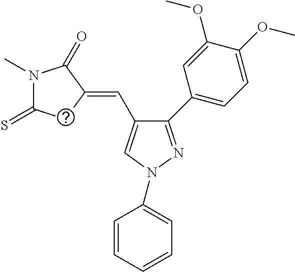 [SMILES: COc1ccc(-c2nn(-c3ccccc3)cc2/C=C2\SC(=S)N(C)C2=O)cc1OC]

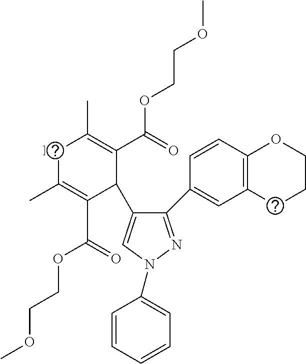 [SMILES: COCCOC(=O)C1=C(C)NC(C)=C(C(=O)OCCOC)C1c1cn(-c2ccccc2)nc1-c1ccc2c(c1)OCCO2]

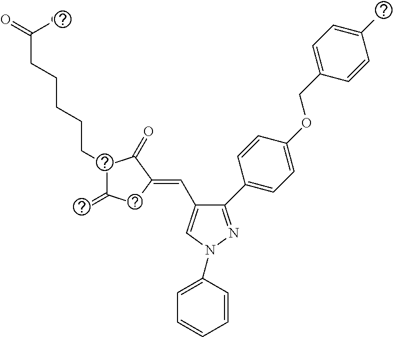 [SMILES: O=C(O)CCCCCN1C(=O)/C(=C/c2cn(-c3ccccc3)nc2-c2ccc(OCc3ccc(F)cc3)cc2)SC1=S]

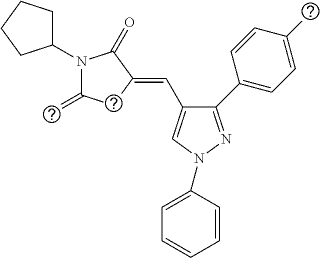 [SMILES: O=C1/C(=C/c2cn(-c3ccccc3)nc2-c2ccc(F)cc2)SC(=S)N1C1CCCC1]

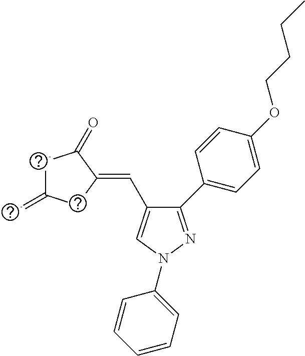 [SMILES: CCCCOc1ccc(-c2nn(-c3ccccc3)cc2/C=C2\SC(=N)NC2=O)cc1]

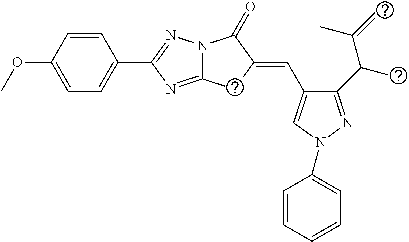 [SMILES: C=C(C)C(C)c1nn(-c2ccccc2)cc1/C=c1\sc2nc(-c3ccc(OC)cc3)nn2c1=O]

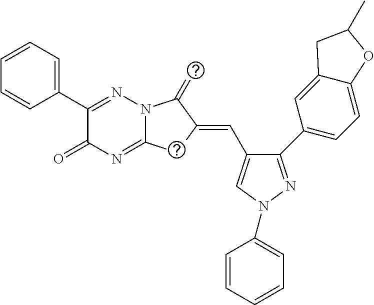 [SMILES: CC1Cc2cc(-c3nn(-c4ccccc4)cc3/C=c3\sc4nc(=O)c(-c5ccccc5)nn4c3=O)ccc2O1]

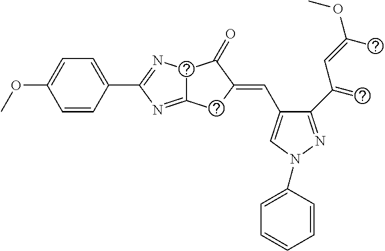 [SMILES: C=C(/C=C(\C)OC)c1nn(-c2ccccc2)cc1/C=c1\sc2nc(-c3ccc(OC)cc3)nn2c1=O]

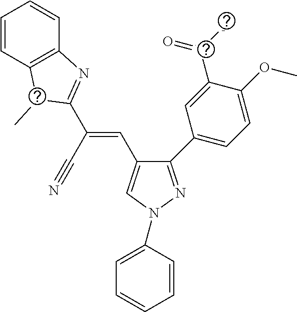

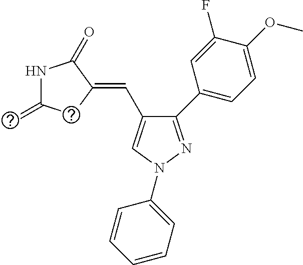 [SMILES: COc1ccc(-c2nn(-c3ccccc3)cc2/C=C2\SC(=S)NC2=O)cc1F]

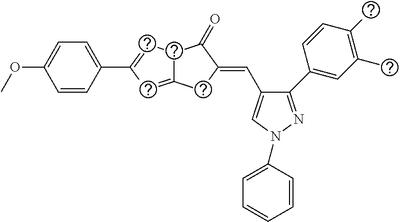

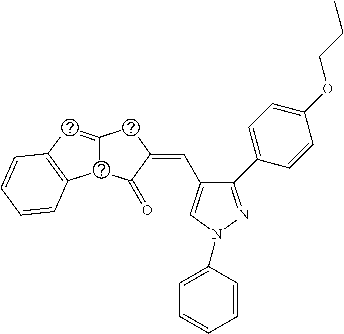 [SMILES: CCCOc1ccc(-c2nn(-c3ccccc3)cc2/C=c2/sc3nc4ccccc4n3c2=O)cc1]

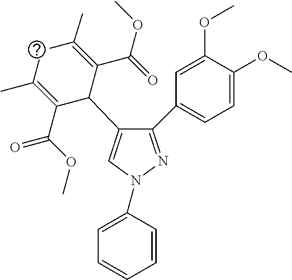

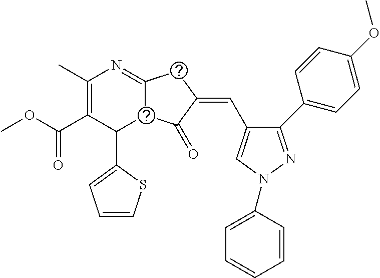 [SMILES: COC(=O)C1=C(C)N=c2s/c(=C/c3cn(-c4ccccc4)nc3-c3ccc(OC)cc3)c(=O)n2C1c1cccs1]

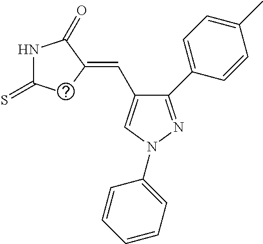 [SMILES: Cc1ccc(-c2nn(-c3ccccc3)cc2/C=C2\SC(=S)NC2=O)cc1]

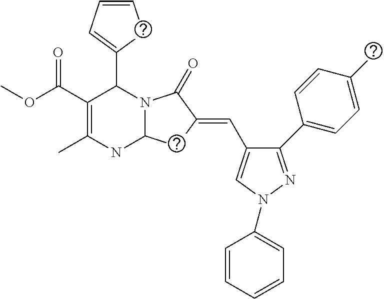 [SMILES: COC(=O)C1=C(C)NC2S/C(=C\c3cn(-c4ccccc4)nc3-c3ccc(F)cc3)C(=O)N2C1c1ccc[nH]1]

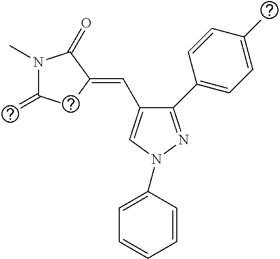 [SMILES: CN1C(=O)/C(=C/c2cn(-c3ccccc3)nc2-c2ccc(F)cc2)SC1=S]

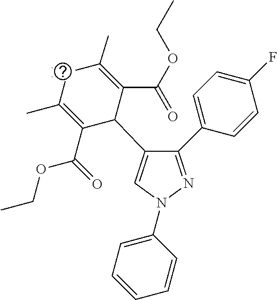 [SMILES: CCOC(=O)C1=C(C)NC(C)=C(C(=O)OCC)C1c1cn(-c2ccccc2)nc1-c1ccc(F)cc1]

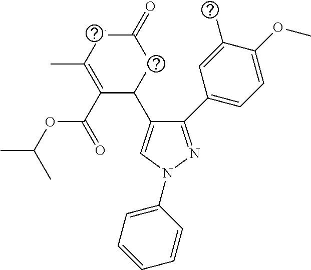 [SMILES: COc1ccc(-c2nn(-c3ccccc3)cc2C2NC(=O)NC(C)=C2C(=O)OC(C)C)cc1Br]

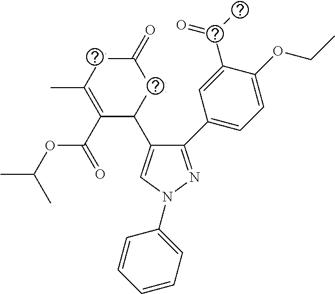

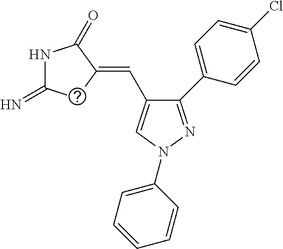 [SMILES: N=C1NC(=O)/C(=C/c2cn(-c3ccccc3)nc2-c2ccc(Cl)cc2)S1]

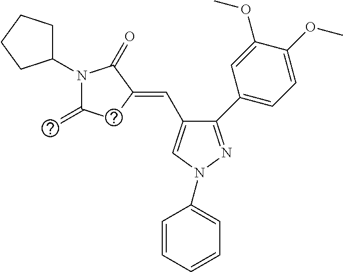 [SMILES: COc1ccc(-c2nn(-c3ccccc3)cc2/C=C2\SC(=S)N(C3CCCC3)C2=O)cc1OC]

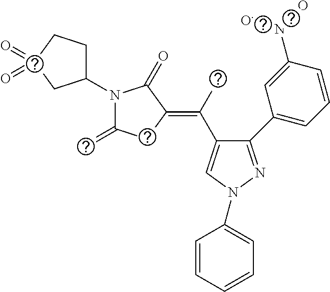 [SMILES: O=C1/C(=C/c2cn(-c3ccccc3)nc2-c2cccc(N([O-])O)c2)SC(=S)N1C1CCS(=O)(=O)C1]

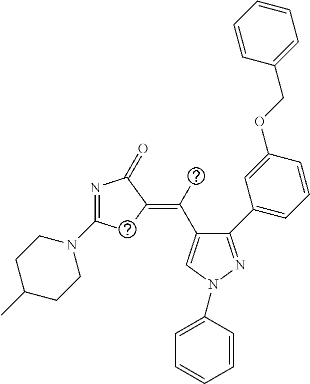 [SMILES: CC1CCN(C2=NC(=O)/C(=C/c3cn(-c4ccccc4)nc3-c3cccc(OCc4ccccc4)c3)S2)CC1]

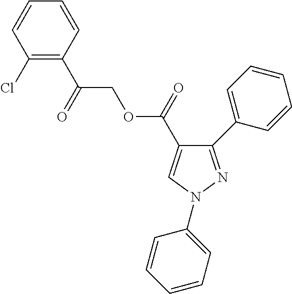 [SMILES: O=C(COC(=O)c1cn(-c2ccccc2)nc1-c1ccccc1)c1ccccc1Cl]

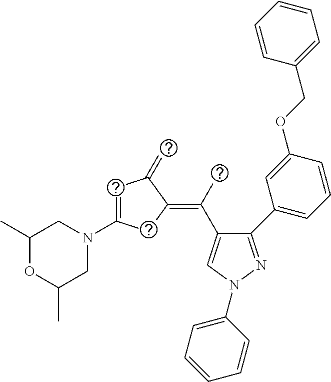 [SMILES: CC1CN(C2=NC(=O)/C(=C/c3cn(-c4ccccc4)nc3-c3cccc(OCc4ccccc4)c3)S2)CC(C)O1]

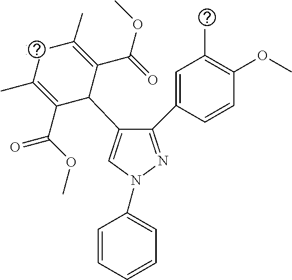

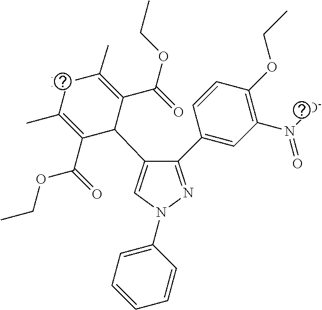

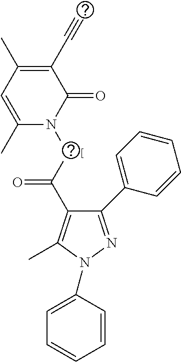 [SMILES: Cc1cc(C)n(NC(=O)c2c(-c3ccccc3)nn(-c3ccccc3)c2C)c(=O)c1C#N]

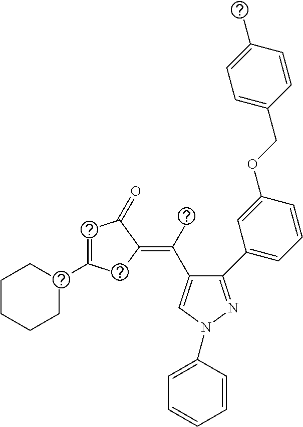 [SMILES: O=C1N=C(N2CCCCC2)S/C1=C\c1cn(-c2ccccc2)nc1-c1cccc(OCc2ccc(F)cc2)c1]

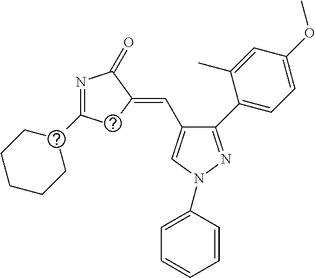 [SMILES: COc1ccc(-c2nn(-c3ccccc3)cc2/C=C2\SC(N3CCCCC3)=NC2=O)c(C)c1]

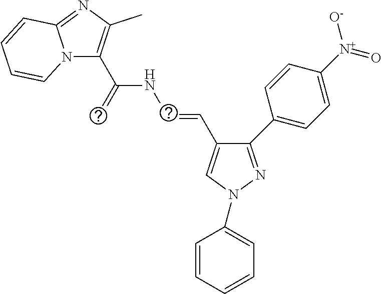 [SMILES: Cc1nc2ccccn2c1C(=O)N/N=C/c1cn(-c2ccccc2)nc1-c1ccc([N+](=O)[O-])cc1]

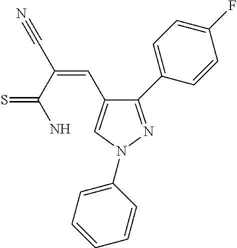 [SMILES: N#C/C(=C/c1cn(-c2ccccc2)nc1-c1ccc(F)cc1)C(N)=S]

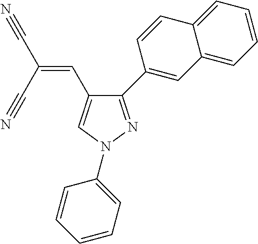 [SMILES: N#CC(C#N)=Cc1cn(-c2ccccc2)nc1-c1ccc2ccccc2c1]

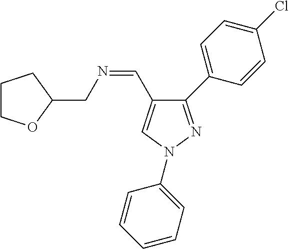 [SMILES: Clc1ccc(-c2nn(-c3ccccc3)cc2/C=N\CC2CCCO2)cc1]

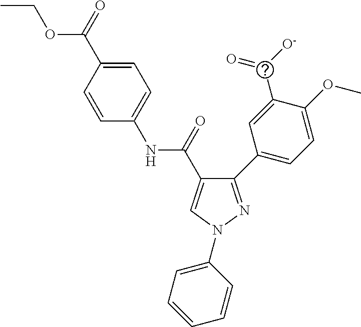

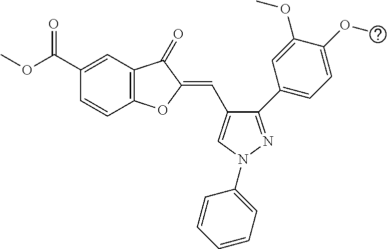

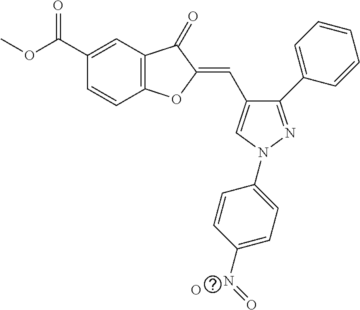

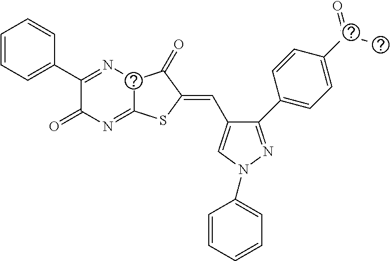

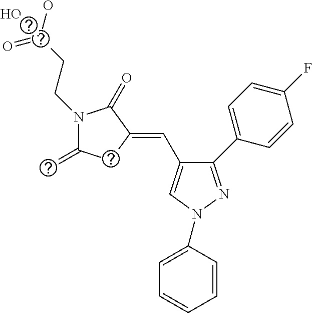

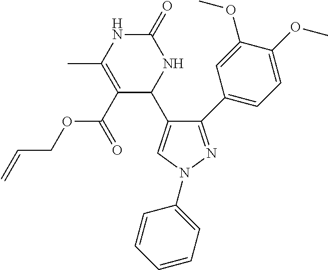 [SMILES: C=CCOC(=O)C1=C(C)NC(=O)NC1c1cn(-c2ccccc2)nc1-c1ccc(OC)c(OC)c1]

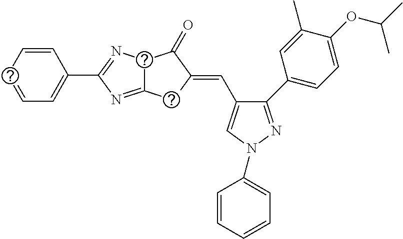 [SMILES: Cc1cc(-c2nn(-c3ccccc3)cc2/C=c2\sc3nc(-c4ccncc4)nn3c2=O)ccc1OC(C)C]

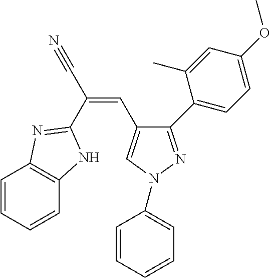 [SMILES: COc1ccc(-c2nn(-c3ccccc3)cc2/C=C(/C#N)c2nc3ccccc3[nH]2)c(C)c1]

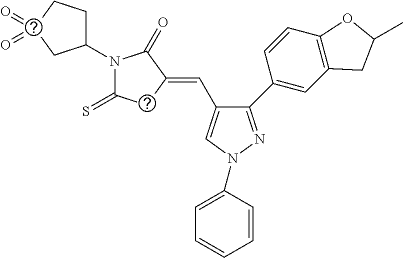 [SMILES: CC1Cc2cc(-c3nn(-c4ccccc4)cc3/C=C3\SC(=S)N(C4CCS(=O)(=O)C4)C3=O)ccc2O1]

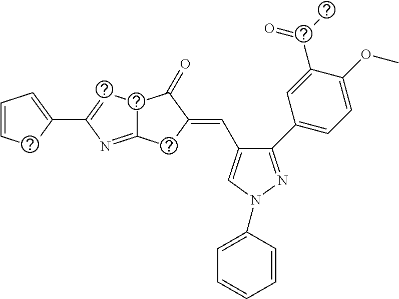

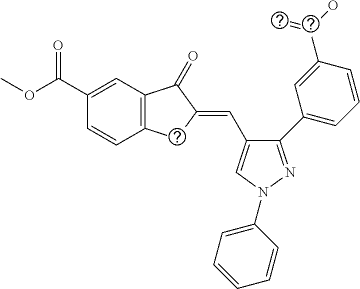

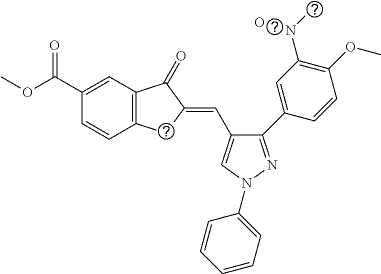

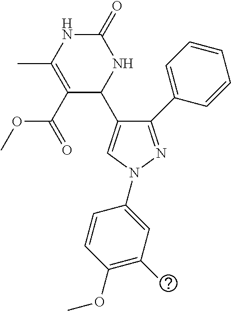 [SMILES: COC(=O)C1=C(C)NC(=O)NC1c1cn(-c2ccc(OC)c(Br)c2)nc1-c1ccccc1]

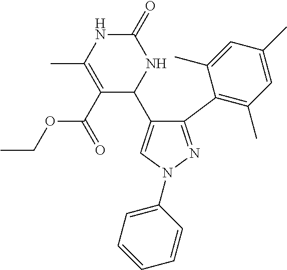 [SMILES: CCOC(=O)C1=C(C)NC(=O)NC1c1cn(-c2ccccc2)nc1-c1c(C)cc(C)cc1C]

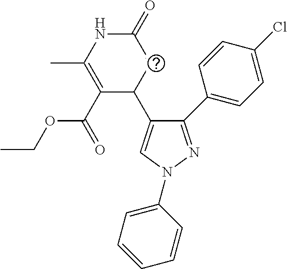 [SMILES: CCOC(=O)C1=C(C)NC(=O)NC1c1cn(-c2ccccc2)nc1-c1ccc(Cl)cc1]

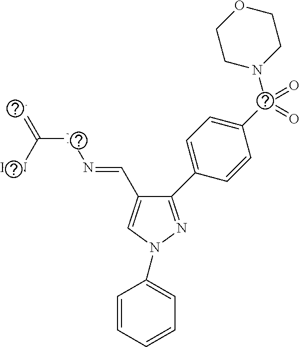 [SMILES: N=C(N)N/N=C/c1cn(-c2ccccc2)nc1-c1ccc(S(=O)(=O)N2CCOCC2)cc1]

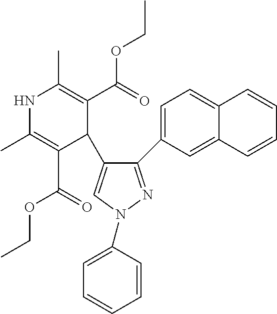 [SMILES: CCOC(=O)C1=C(C)NC(C)=C(C(=O)OCC)C1c1cn(-c2ccccc2)nc1-c1ccc2ccccc2c1]

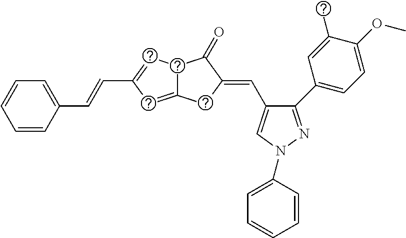 [SMILES: COc1ccc(-c2nn(-c3ccccc3)cc2/C=c2\sc3nc(/C=C/c4ccccc4)nn3c2=O)cc1[O-]]

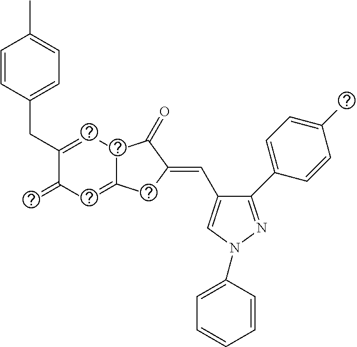 [SMILES: Cc1ccc(Cc2nn3c(=O)/c(=C/c4cn(-c5ccccc5)nc4-c4ccc(Br)cc4)sc3nc2=O)cc1]

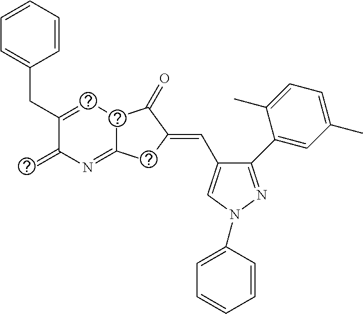 [SMILES: Cc1ccc(C)c(-c2nn(-c3ccccc3)cc2/C=c2\sc3nc(=O)c(Cc4ccccc4)nn3c2=O)c1]

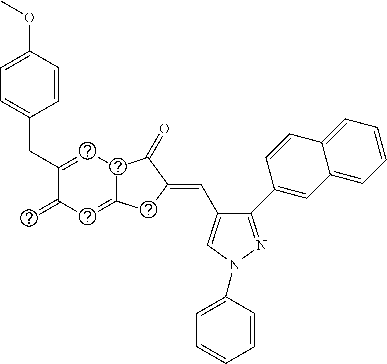 [SMILES: COc1ccc(Cc2nn3c(=O)/c(=C/c4cn(-c5ccccc5)nc4-c4ccc5ccccc5c4)sc3nc2=O)cc1]

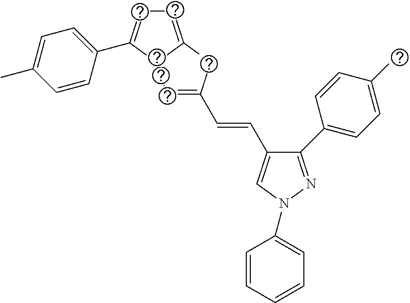 [SMILES: Cc1ccc(-c2nnc3sc(/C=C/c4cn(-c5ccccc5)nc4-c4ccc(F)cc4)nn23)cc1]

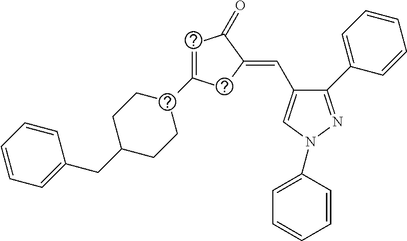 [SMILES: O=C1N=C(N2CCC(Cc3ccccc3)CC2)S/C1=C\c1cn(-c2ccccc2)nc1-c1ccccc1]

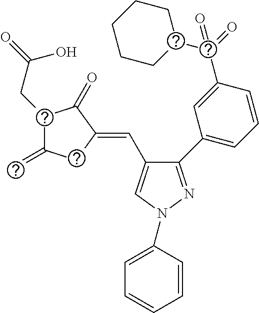 [SMILES: O=C(O)CN1C(=O)/C(=C/c2cn(-c3ccccc3)nc2-c2cccc(S(=O)(=O)N3CCCCC3)c2)SC1=S]

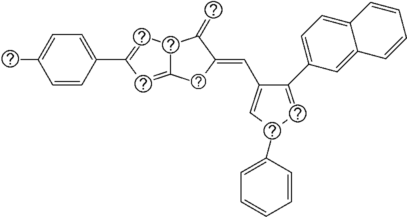

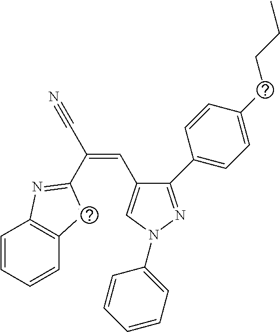 [SMILES: CCCNc1ccc(-c2nn(-c3ccccc3)cc2/C=C(/C#N)c2nc3ccccc3[nH]2)cc1]

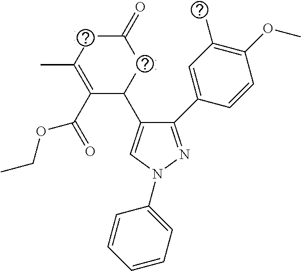 [SMILES: CCOC(=O)C1=C(C)NC(=O)NC1c1cn(-c2ccccc2)nc1-c1ccc(OC)c(Br)c1]

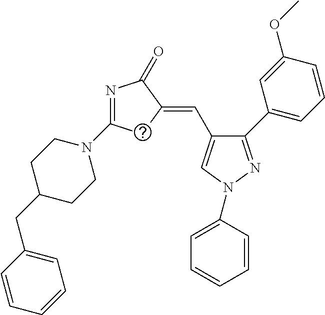 [SMILES: COc1cccc(-c2nn(-c3ccccc3)cc2/C=C2\SC(N3CCC(Cc4ccccc4)CC3)=NC2=O)c1]

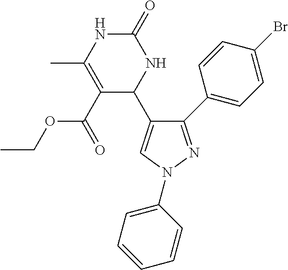 [SMILES: CCOC(=O)C1=C(C)NC(=O)NC1c1cn(-c2ccccc2)nc1-c1ccc(Br)cc1]

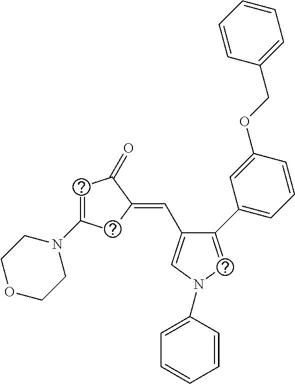 [SMILES: O=C1N=C(N2CCOCC2)S/C1=C\c1cn(-c2ccccc2)nc1-c1cccc(OCc2ccccc2)c1]

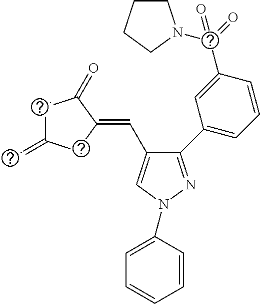 [SMILES: N=C1NC(=O)/C(=C/c2cn(-c3ccccc3)nc2-c2cccc(S(=O)(=O)N3CCCC3)c2)S1]

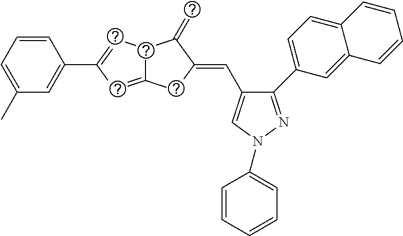 [SMILES: Cc1cccc(-c2nc3s/c(=C\c4cn(-c5ccccc5)nc4-c4ccc5ccccc5c4)c(=O)n3n2)c1]

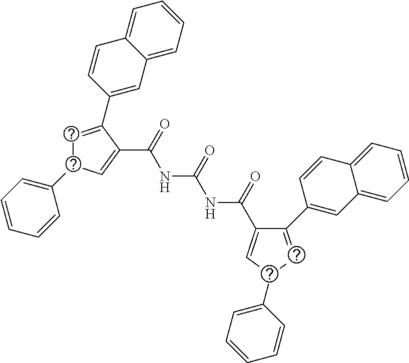 [SMILES: O=C(NC(=O)c1cn(-c2ccccc2)nc1-c1ccc2ccccc2c1)NC(=O)c1cn(-c2ccccc2)nc1-c1ccc2ccccc2c1]

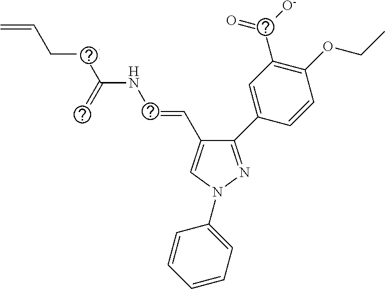

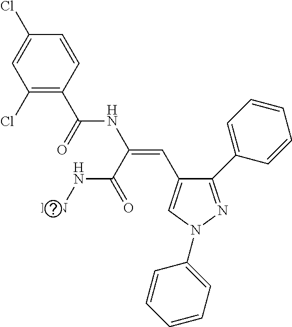 [SMILES: NNC(=O)/C(=C\c1cn(-c2ccccc2)nc1-c1ccccc1)NC(=O)c1ccc(Cl)cc1Cl]

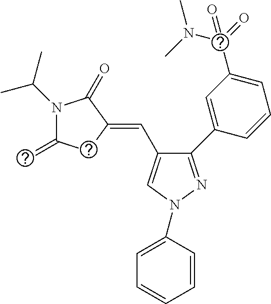 [SMILES: CC(C)N1C(=O)/C(=C/c2cn(-c3ccccc3)nc2-c2cccc(S(=O)(=O)N(C)C)c2)SC1=S]

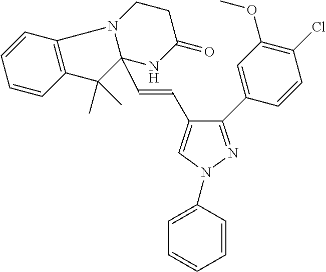 [SMILES: COc1cc(-c2nn(-c3ccccc3)cc2/C=C/C23NC(=O)CCN2c2ccccc2C3(C)C)ccc1Cl]

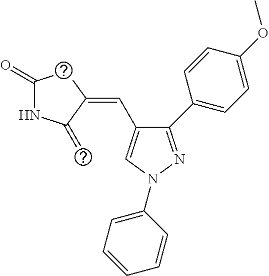 [SMILES: COc1ccc(-c2nn(-c3ccccc3)cc2/C=C2/SC(=O)NC2=S)cc1]

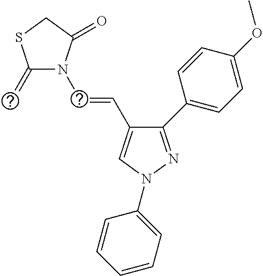 [SMILES: COc1ccc(-c2nn(-c3ccccc3)cc2/C=N/N2C(=O)CSC2=S)cc1]

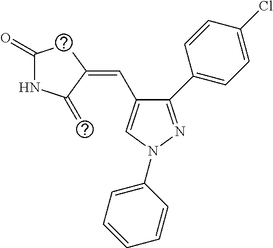 [SMILES: O=C1NC(=S)/C(=C\c2cn(-c3ccccc3)nc2-c2ccc(Cl)cc2)S1]

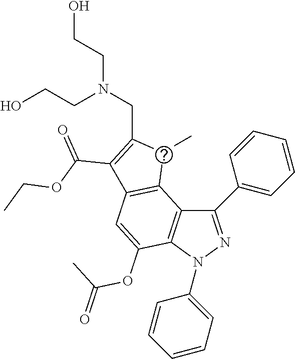 [SMILES: CCOC(=O)c1c(CN(CCO)CCO)n(C)c2c1cc(OC(C)=O)c1c2c(-c2ccccc2)nn1-c1ccccc1]

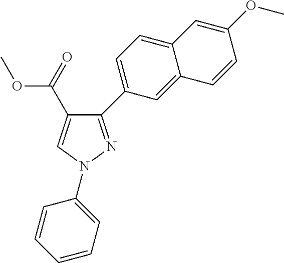

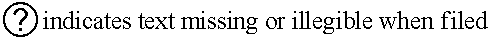

### REFERENCES

Ahmed, S. A., Gogal, R. M., Jr., and Walsh, J. E. 1994. A new rapid and simple non-radioactive assay to monitor and determine the proliferation of lymphocytes: an alternative to [3H]thymidine incorporation assay. J. Immunol. Methods 170(2): 211-224.
Alley, M. C., Scudiero, D. A., Monks, A., Hursey, M. L., Czerwinski, M. J., Fine, D. L., Abbott, B. J., Mayo, J. G., Shoemaker, R. H., and Boyd, M. R. 1988. Feasibility of drug screening with panels of human tumor cell lines using a microculture tetrazolium assay. Cancer Res 48(3): 589-601.
Basu, T. N., Gutmann, D. H., Fletcher, J. A., Glover, T. W., Collins, F. S., and Downward, J. 1992. Aberrant regulation of ras proteins in malignant tumour cells from type 1 neurofibromatosis patients. Nature 356(6371): 713-715.
Dahlberg, W. K., Little, J. B., Fletcher, J. A., Suit, H. D., and Okunieff, P. 1993. Radiosensitivity in vitro of human soft tissue sarcoma cell lines and skin fibroblasts derived from the same patients. Int J Radiat Biol 63(2): 191-198.
De Raedt, T., Brems, H., Wolkenstein, P., Vidaud, D., Pilotti, S., Perrone, F., Mautner, V., Frahm, S., Sciot, R., and Legius, E. 2003. Elevated risk for MPNST in NF1 microdeletion patients. Am J Hum Genet. 72(5):1288-1292.
DeClue, J. E., Papageorge, A. G., Fletcher, J. A., Diehl, S. R., Ratner, N., Vass, W. C., and Lowy, D. R. 1992. Abnormal regulation of mammalian p2lras contributes to malignant tumor growth in von Recklinghausen (type 1) neurofibromatosis. Cell 69(2): 265-273.
Evans, D. G., Baser, M. E., McGaughran, J., Sharif, S., Howard, E., and Moran, A. 2002. Malignant peripheral nerve sheath tumours in neurofibromatosis 1. J Med Genet. 39(5): 311-314.
Fletcher, J. A., Kozakewich, H. P., Hoffer, F. A., Lage, J. M., Weidner, N., Tepper, R., Pinkus, G. S., Morton, C. C., and Corson, J. M. 1991. Diagnostic relevance of clonal cytogenetic aberrations in malignant soft-tissue tumors. N Engl J Med 324(7): 436-442.
Frahm, S., Mautner, V. F., Brems, H., Legius, E., Debiec-Rychter, M., Friedrich, R. E., Knofel, W. T., Peiper, M., and Kluwe, L. 2004. Genetic and phenotypic characterization of tumor cells derived from malignant peripheral nerve sheath tumors of neurofibromatosis type 1 patients. Neurobiol Dis 16(1): 85-91.
Glover, T. W., Stein, C. K., Legius, E., Andersen, L. B., Brereton, A., and Johnson, S. 1991. Molecular and cytogenetic analysis of tumors in von Recklinghausen neurofibromatosis. Genes Chromosomes Cancer 3(1): 62-70.
Mahller, Y. Y., Rangwala, F. Ratner, N. and Cripe, T. P. (2006) Malignant Peripheral Nerve Sheath Tumors with High and Low Ras are Permissive for Oncolytic Herpes Simplex Virus Mutants, Pediatric Blood and Cancer, 46(7):745-54.
Mahller Y Y, Vaikunth S S, Currier M A, Miller S J, Ripberger M C, Hsu Y-H, Mehrian-Shai R, Collins M H, Crombleholme T M, Ratner N, Cripe T P. (2007) Oncolytic HSV and Erlotinib Inhibit Tumor Growth and Angiogenesis in a Novel Malignant Peripheral Nerve Sheath Tumor Xenograft Model. Molecular Therapy, 15:279-286.
Miller, S J, Rangwala, F, Williams, J Kong, S, Jegga, A, Aronow, B, Frahm, 5, Kluwe, L, Mautner, V, Upadhyaya, M, Muir, D, Wallace, M, Hagen, J, Quelle, D E, Watson, M, Perry, A, Gutmann, D H, and Ratner, N. (2006) Large-scale molecular comparison of human Schwann cells to malignant peripheral nerve sheath tumor cell lines and tissues, Cancer Research, 66(5):2584-91.
Nagashima, Y., Ohaki, Y., Tanaka, Y., Sumino, K., Funabiki, T., Okuyama, T., Watanabe, S., Umeda, M., and Misugi, K. 1990. Establishment of an epithelioid malignant schwannoma cell line (YST-1). Virchows Arch B Cell Pathol Incl Mol Pathol 59(5): 321-327.
Rasmussen, S. A. and Friedman, J. M. 2000. NF1 gene and neurofibromatosis 1. Am J Epidemiol 151(1): 33-40.
Ratner, N., Jon P. Williams, J. P., Kordich, J. J., and Kim, H. A. (2005) Schwann cell preparation from single mouse embryos: Analyses of neurofibromin function in Schwann cells. Meth. Enzymol 407:22-33 in Regulators and Effectors of Small GTPases, Part D: Ras Family. Ed. Balch, Der, C. J., and Hall, A.
Reynolds, J. E., Fletcher, J. A., Lytle, C. H., Nie, L., Morton, C. C., and Diehl, S. R. 1992. Molecular characterization of a 17q11.2 translocation in a malignant schwannoma cell line. Hum Genet. 90(4): 450-456.
Wu, R., Lopez-Correa, C., Rutkowski, J. L., Baumbach, L. L., Glover, T. W., and Legius, E. 1999. Germline mutations in NF1 patients with malignancies. Genes Chromosomes Cancer 26(4): 376-380.
Zoller, M. E., Rembeck, B., Oden, A., Samuelsson, M., and Angervall, L. 1997. Malignant and benign tumors in patients with neurofibromatosis type 1 in a defined Swedish population. Cancer 79(11): 2125-2131.

What is claimed is:

1. A method of treating a disorder associated with Ras deregulation or dysregulation comprising the step of administering a pharmaceutical composition comprising Formula IV: 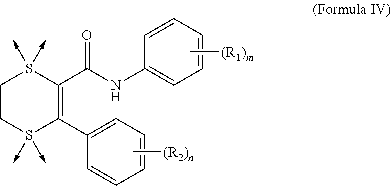
or a pharmaceutically acceptable salt thereof, and a pharmaceutically-acceptable carrier; wherein:
each R₁ and R₂ is independently selected from F, Cl, Br, cyano, methyl, ethyl, and methoxy; and
m and n are independently an integer from 0 to 5.

2. The method of claim 1, wherein R₁ is selected from any one of F, Cl, ethyl, and methoxy, and n is 0, and m is an integer from 1 to 5.

3. The method of claim 1 wherein R₂ is selected from any one of F, Cl, ethyl, and methoxy, and m is 0, and n is an integer from 1 to 5.

4. The method of claim 1 wherein said compound is 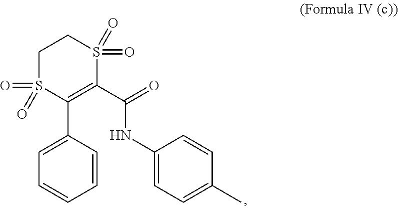 [SMILES: Cc1ccc(NC(=O)C2=C(c3ccccc3)S(=O)(=O)CCS2(=O)=O)cc1]
or a pharmaceutically acceptable salt thereof.

5. The method of claim 1 wherein said compound is 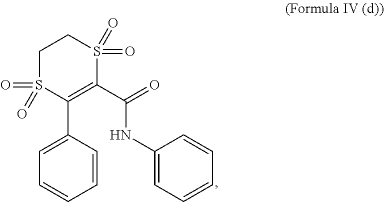 [SMILES: O=C(Nc1ccccc1)C1=C(c2ccccc2)S(=O)(=O)CCS1(=O)=O]
or a pharmaceutically acceptable salt thereof.

6. The method of claim 1 wherein said disorder associated with Ras deregulation or dysregulation comprises a proliferative disorder.

7. The method of claim 1 wherein said disorder associated with Ras deregulation or dysregulation comprises cancer.

8. The method of claim 1 wherein said disorder associated with Ras deregulation or dysregulation comprises Neurofibromatosis Type 1

9. The method of claim 1, wherein said disorder associated with Ras deregulation or dysregulation disorder comprises a disease state that results from a mutation or loss of function in an NF1 gene, wherein said disease state comprises a cancer.

10. A method according to claim 9 wherein said cancer is selected from neuroblastoma, lung adenocarcinoma, squamous cell carcinoma, glioblastomas, pancreatic cancer; colon cancer; lung cancer; neurofibromas, malignant peripheral nerve sheath tumors, optic gliomas, Schwannomas, gliomas, leukemias, pheochromocytomas, pancreatic adenocarcinoma and combinations thereof.

11. A method according to claim 1 wherein said disorder associated with Ras deregulation or dysregulation comprises a disorder caused by Candida albicans.

12. A method of treating a disorder associated with Ras deregulation or dysregulation comprising the step of administering a pharmaceutical composition comprising the formula: 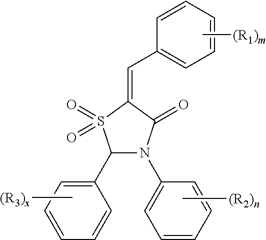
or a pharmaceutically acceptable salt thereof, and a pharmaceutically-acceptable carrier; wherein:
each R₁, R₂, and R₃ is independently selected from F, Cl, Br, cyano, methyl, ethyl, and methoxy; and
m, n, and x are independently an integer from 0 to 5.

13. The method of claim 12, wherein R₁ is selected from any one of F, Cl, ethyl, and methoxy, and m is an integer from 1 to 5.

14. The method of claim 12, wherein R₂ is selected from any one of F, Cl, ethyl, and methoxy, and x is 0, and n is an integer from 1 to 5.

15. The method of claim 12, wherein R₃ is selected from any one of F, Cl, ethyl, and methoxy, and n is 0, and x is an integer from 1 to 5.

16. The method of claim 12, wherein said compound is selected from 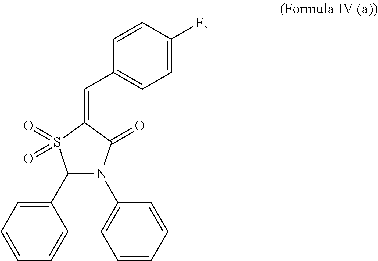 [SMILES: O=C1/C(=C\c2ccc(F)cc2)S(=O)(=O)C(c2ccccc2)N1c1ccccc1]
or a pharmaceutically acceptable salt thereof; 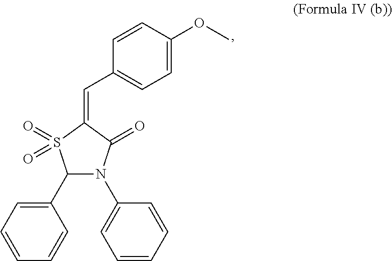 [SMILES: COc1ccc(/C=C2\C(=O)N(c3ccccc3)C(c3ccccc3)S2(=O)=O)cc1]
or a pharmaceutically acceptable salt thereof.

17. The method of claim 12 wherein said disorder associated with Ras deregulation or dysregulation comprises a proliferative disorder

18. A method according to claim 12 wherein said disorder associated with Ras deregulation or dysregulation comprises cancer.

19. A method according to claim 12 wherein said disorder associated with Ras deregulation or dysregulation comprises Neurofibromatosis Type 1

20. The method of claim 12, wherein said disorder associated with Ras deregulation or dysregulation disorder comprises a disease state that results from a mutation or loss of function in an NF1 gene, wherein said disease state comprises a cancer.

21. A method according to claim 18, wherein said cancer is selected from neuroblastoma, lung adenocarcinoma, squamous cell carcinoma, glioblastomas, pancreatic cancer; colon cancer; lung cancer; neurofibromas, malignant peripheral nerve sheath tumors, optic gliomas, Schwannomas, gliomas, leukemias, pheochromocytomas, pancreatic adenocarcinoma and combinations thereof.

22. A method according to claim 12 wherein said disorder associated with Ras deregulation or dysregulation comprises a disorder caused by Candida albicans.

23. A method of treating a disorder associated with Ras deregulation or dysregulation comprising the step of administering a therapeutically effective amount of a compound selected from Formula IV, Formula IV(a), Formula IV(b), Formula IV(c), Formula IV(d), and combinations thereof.

24. The method of claim 23 wherein said compound comprises Formula IV(c), or a pharmaceutically acceptable salt thereof.

25. The method of claim 23 wherein said disorder associated with Ras deregulation or dysregulation is selected from neuroblastoma, lung adenocarcinoma, squamous cell carcinoma, glioblastomas, pancreatic cancer; colon cancer; lung cancer; neurofibromas, malignant peripheral nerve sheath tumors, optic gliomas, Schwannomas, gliomas, leukemias, pheochromocytomas, pancreatic adenocarcinoma, ovarian cancer, or combinations thereof.
